SEQUENCE LISTING (2 sequences)

SEQ ID NO: 1 - DNA, 39 bp, synthetic construct
  source 1..39: mol_type = other DNA
ggagaagtctgccgttactgccctgtggggcaaggtgaa

SEQ ID NO: 2 - DNA, 39 bp, synthetic construct
  source 1..39: mol_type = other DNA
ggagaagtnnnnngttactagtctgtnnnnnaaggtgaa

(19) United States
(12) Patent Application Publication
(10) Pub. No.: US 2026/0327716 A1
(43) Pub. Date: Oct. 22, 2026

(54) Tumor-Naïve Tumor Fraction Measurements at Sub-molecule Limit of Detection

(71) Applicant: BillionToOne, Inc., Menlo Park, CA (US)

(72) Inventors: David Tsao, San Carlos, CA (US); Lee Davis McDaniel, JR., Milpitas, CA (US)

(21) Appl. No.: 19/650,199

(22) Filed: Apr. 16, 2026

Related U.S. Application Data
(60) Provisional application No. 63/789,918, filed on Apr. 16, 2025.
(60) Provisional application No. 63/824,440, filed on Jun. 16, 2025.

(51) Int. Cl. C12Q 1/6886; C12Q 1/6806; C12Q 1/6827; C12Q 1/6851; C12Q 1/6869

(52) U.S. Cl. CPC C12Q 1/6886; C12Q 1/6806; C12Q 1/6827; C12Q 1/6851; C12Q 1/6869; C12Q 2600/156

(57) ABSTRACT

Methods are provided for determining tumor fraction in cell-free DNA (cfDNA) without prior knowledge of tumor-specific genomic alterations. The methods involve determining a mean number of methylated molecules at each locus of a set of target loci and performing a statistical analysis that exclude loci with zero methylated molecules to determine an estimate of the true mean number of methylated molecules per locus. Specificity may be improved by excluding loci that exhibit methylation in white blood cell DNA extracted from the buffy coat, and accuracy may be further enhanced through background subtraction and grouping loci into haplotype blocks. Applications include monitoring cancer progression, treatment response, and detecting tumor heterogeneity.

### CROSS REFERENCE TO RELATED APPLICATIONS

The present application claims the benefit of U.S. Provisional Patent Application No. 63/789,918 filed Apr. 16, 2025, and U.S. Provisional Paten Application No. 63/824,440, filed Jun. 16, 2025, each of which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “38227-66202_P16WO_SeqList”. The XML file is 2,674 bytes, created on Apr. 16, 2026, and is being submitted electronically via USPTO Patent Center.

### BACKGROUND

While novel cancer treatments continue to be developed at an unprecedented pace, assessing whether cancer treatment is effective for a particular patient remains relatively cumbersome and qualitative. Imaging is the gold standard for measuring the state of a cancer; however, PET/CT and CT scans impart harmful radioactive dose to the patient, and these imaging facilities may be located far from where the patient lives. Imaging sessions are scheduled several months apart, which limits how quickly oncologists can get a sense of how the cancer is responding to a treatment. Qualitative metrics such as physical exam and patient reported symptoms are often confounded by treatment side effects and therefore are not sufficiently accurate for assessing treatment efficacy. Nevertheless, oncologists want to assess the extent to which a patient's cancer is responding to treatment to inform the treatment plan, such as whether the patient should continue with existing therapy or switch to a new therapy plan.

Non-invasive liquid biopsies that assay the cell-free DNA (cfDNA) have been developed to quantify the levels of circulating tumor DNA (ctDNA) from a blood sample. Cell-free DNA includes ctDNA) and DNA shed by non-tumor cells such as immune and endothelial cells. Tumor fraction (TF) measures the proportion of ctDNA present in cfDNA. Changes in TF over time may suggest clinically relevant transitions in tumor size, tumor biology, or both.

Given the molecule sampling challenges associated with assaying somatic mutations, methylation has been explored as an alternative biomarker for ctDNA. Methylation has long been shown to be a strong, consistent, and genomically widespread biomarker for cancer. However, while methylation signal is much more abundant compared to that from somatic mutations, quantifying the amount of methylation accurately and precisely is a challenge. Methylation-specific qPCR is a commonly used method; however, because of the exponential nature of the assay, qPCR Ct measurements have high CV (typically in the 10s of percent). Improving the accuracy with multiple replicates is challenging given the limited sample amount. In addition, non-tumor DNA can contribute background methylation signal, complicating the task of quantifying the signal that belongs to ctDNA. Furthermore, sampling multiple locations for methylation would help improve assay performance, but interrogating multiple loci using qPCR is difficult and time consuming.

Thus, there is a need for assays that probe for detectable biomarkers that provide clinically relevant information about tumor size and progression. DNA sequencing of circulating tumor DNA (ctDNA) found in blood plasma has recently become the standard of care for cancer patients. These so-called liquid biopsy assays detect genomic alterations in the tumor from a non-invasive blood sample, and they are a convenient and expeditious way to profile a tumor for somatic alterations that can be treated with a targeted therapy. One challenge for these assays is the fact that ctDNA is exceedingly rare in blood and is often present at a proportion of <0.1% of the total cell-free DNA. An effective liquid biopsy therefore has an ability to detect variants with a false positive rate far below 0.1% allele fraction. Since a typical liquid biopsy might be looking for small nucleotide variants, such as Single Nucleotide Variants (SNVs) or indels over >100 kilobase of genomic content, the per-base specificity may be >99.999% to provide confidence that there are no false positives in a patient's liquid biopsy test results.

Quantification of circulating tumor DNA is increasingly used to monitor disease progression in multiple clinical settings. In early-stage cancers, detection and quantification of ctDNA is used for adjuvant therapy monitoring and surveillance. In late-stage cancer, ctDNA can be used to assess treatment response and monitor for cancer progression.

Current approaches for ctDNA detection and quantification often rely on tumor tissue samples to identify tumor-specific genomic variants. So-called “bespoke” ctDNA assays are created by first determining a set of tumor-specific genomic variants through analyzing a tumor tissue sample. A custom ctDNA assay is then created for each patient based on the alterations found in the tissue.

However, the bespoke approach suffers from logistical challenges associated with acquiring and analyzing tissue, which can increase assay failure rates and delay results. Furthermore, such assays may not capture details of cancer evolution, because they are designed against the initial tumor sample. Therefore, bespoke assays may miss cancer progression events associated with therapy resistance and failure, metastasis and spread to distant sites, and acquisition of new genetic features.

There is a need for ctDNA assays that do not rely on tumor tissue. Tumor biopsies are obtained infrequently, typically only at the point of first diagnosis. Over time, the initial tumor biopsy may no longer be representative of a heterogeneous cancer that (1) partially responds to therapy, (2) acquires therapy-resistant cancer subclones, or (3) metastasizes to distal anatomical sites. Furthermore, a tumor biopsy is inherently a localized sample of cancer cells, and it may not be reflective of other cancerous lesions.

Despite the advantages of measuring ctDNA tumor fraction independently of a tumor tissue sample, there are several inherent challenges to overcome. There may be non-cancer specific signals in cell-free DNA, including clonal hematopoiesis and epigenetic drift, which are somatic genomic and epigenomic alterations, respectively. A particularly significant challenge arises at clinically relevant tumor fractions, which are often very low (<0.1%). At such low fractions, Poisson sampling dictates that most loci will detect zero tumor molecules even when the alteration is truly present in the tumor. For example, at a 0.1% tumor fraction with 1000 genomic equivalents of input DNA, a given locus has only a 63% probability of containing zero tumor-derived molecules, while at 0.01% tumor fraction, this probability rises to 90%. This sampling limitation severely confounds traditional analysis methods that rely on direct detection of alterations across multiple loci.

### SUMMARY

The following disclosure describes methods for detecting and quantifying circulating tumor DNA (ctDNA) in biological samples. More specifically, methods for determining tumor fraction in cell-free DNA samples without prior knowledge of tumor-specific genomic alterations.

Tumor Fraction is often estimated by summary statistics on somatic variants such as the sum or maximum variant allele fraction (VAF). The Northstar Response (NSR) assay quantifies ctDNA by accurately and precisely counting methylated cfDNA molecules across over 500 loci which are uniquely methylated in tumor tissue but not in any non-cancerous cells. Conversely, the Northstar Select (NSS) assay identifies somatic mutations, copy number changes, and aneuploidy events in a panel of clinically relevant genes. NSR estimates TF by counting methylated molecules as opposed to somatic variants, yielding a methylation-based TF (mTF).

VAF-based tumor burden measurement is a well-established cancer biomarker that has demonstrated clinical validity and utility in diverse settings. For example, longitudinal changes in VAF have been shown to predict treatment response across multiple therapy classes (e.g. immune checkpoint inhibitor and kinase inhibitors), cancerous tissues, and assays from different laboratory providers.

In various embodiments, an epigenetic-based tumor burden measurement is used in place of a traditional VAF-based approach. NSR quantification of plasma methylated DNA correlates with tumor fraction as estimated by VAF. This correlation can be higher for pathogenic mutations and lower for Clonal Hematopoietic of Indeterminate Potential (CHIP) mutations.

This disclosure generally describes embodiments of an assay that makes clinical determinations based on methylation in cfDNA and specifically an example embodiment in which a tumor methylation score (TMS) that correlates with VAF is used to derive information about a tumor in a patient.

In one embodiment, a method can accurately measure ctDNA tumor fraction without requiring prior tumor tissue analysis. The method uses absolute quantification of hundreds of tumor-specific differentially methylated regions performed in multiplex on a cell-free DNA sample from blood. Statistical methods operating on count data, such as a zero-truncated Poisson (ZTP) distribution analysis, are applied to accurately determine the tumor fraction even when there is less than 1 tumor DNA molecule per locus on average.

In some embodiments, a method for determining tumor fraction in a biological sample comprises: isolating cell-free DNA (cfDNA) and white blood cell DNA (WBC DNA) from a blood sample; subjecting both DNA specimens to bisulfite conversion; performing multiplex PCR to amplify multiple differentially methylated region (DMR) loci; incorporating Quantitative Counting Templates (QCT) during PCR amplification; performing next-generation sequencing (NGS) of the amplified products; determining absolute quantification of methylated DNA molecules at each locus; and applying statistical analysis to the distribution of methylated DNA molecules across the loci to determine tumor fraction. In some embodiments, the method further comprises excluding loci that exhibit methylation in the WBC DNA sample from analysis of the cfDNA sample to improve specificity.

In some embodiments, a method for improving accuracy in tumor fraction determination comprises: grouping multiple loci into haplotype blocks; summing the absolute counts of methylated DNA molecules within each block; and applying zero-truncated Poisson statistical analysis to the blocks rather than individual loci.

Also provided herein is a method of quantifying DNA methylation in a sample, comprising: (a) extracting DNA from the sample; (b) treating the DNA to encode the presence or absence of DNA methylation in the DNA; (c) selectively enriching at least 100 CpG sites (loci); determining an absolute quantification of methylated DNA molecules at each locus; and applying statistical analysis to the distribution of methylated DNA molecules across the loci to determine methylation fraction, wherein the statistical analysis comprises removing loci with zero methylated DNA molecules and calculating the methylation fraction based on a true mean of methylated DNA molecules per locus.

Also provided herein is a method of quantifying the abundance of a DNA modification in a sample, comprising: (a) extracting DNA from the sample; (b) treating the DNA to encode the presence or absence of DNA methylation in the DNA; (c) selectively enriching at least 100 CpG sites (loci); (d) determining an absolute quantification of methylated DNA molecules at each locus; (e) applying statistical analysis to the distribution of methylated DNA molecules across the loci to determine methylation fraction wherein the statistical analysis comprises removing loci with zero methylated DNA molecules and calculating a true mean of methylated DNA molecules per locus; and (f) determining the count of the DNA modifications in the sample based on the true mean of methylated DNA molecules per locus.

Also provided herein is a method of determining tumor fraction in a sample, comprising: (a) extracting DNA from the sample; (b) treating the DNA to encode the presence or absence of DNA methylation in the DNA; (c) selectively enriching at least 100 CpG sites (loci); (d) determining an absolute quantification of methylated DNA molecules at each locus; (e) applying statistical analysis to the distribution of methylated DNA molecules across the loci to determine methylation fraction wherein the statistical analysis comprises removing loci with zero methylated DNA molecules and calculating a true mean of methylated DNA molecules per locus; and (f) determining the tumor fraction in the sample based on the true mean of methylated DNA molecules per locus.

### BRIEF DESCRIPTION OF DRAWINGS

FIG. 1A shows a flowchart representation of a non-limiting embodiment of a method of quantifying DNA methylation in a sample.

FIG. 1B shows a flowchart representation of a non-limiting embodiment of a method of quantifying DNA methylation in sample.

FIG. 2A shows data for the lung cancer detection assay. FIG. 2A shows total methylated molecules for technical replicates of lung cancer contrived samples at various tumor fractions (0% tumor, 0.5% tumor, 1% tumor, 2.5% tumor, and 5% tumor) using the lung cancer assay. 0.5% universal methylated and 5% universal methylated were controls.

FIG. 2B shows data for the lung cancer detection assay in contrived samples from two different lung cancer subjects. FIG. 2B shows total (summed) methylated QE across target loci (n=113). Contrived samples were taken from subjects 18-0901 and 20-0407. Contrived samples were assayed at a 0% tumor fraction and a 5% tumor fraction using the PCR volumes and thermal cycler protocols indicated in FIG. 2B.

FIG. 3 shows concordance values of total QE for each of the samples analyzed in FIG. 2A.

FIG. 4 shows methylation in lung cancer contrived samples with masking and background subtraction for each of the samples analyzed in and FIG. 3. FIG. 4 shows total normalized methylated molecules (y-axis) over the lung cancer contrived samples at various tumor fractions (0% tumor, 0.5% tumor, 1% tumor, 2.5% tumor, and 5% tumor) and controls: 0.5% universal methylated and 5% universal methylated were controls. The data is shown with background subtraction and masking of target loci with high background methylation.

FIG. 5 shows data from simulations of cfDNA tumor signals quantified using somatic mutations and methylation approaches. Top panel shows variant allele fraction (y-axis) over fold gDNA contamination (x-axis)quantified using somatic mutation. Middle panel shows methylated molecules (y-axis) over fold gDNA contamination (x-axis) quantified using methylation with no buffy coat background methylation. Bottom panel shows methylated molecules (y-axis) over fold gDNA contamination (x-axis) quantified using methylation with buffy coat background methylation.

FIG. 6 shows methylation profiles for buffy coats (i.e., 5000 genomic equivalents (g.e.) of buffy coat) isolated from different tubes of the same blood draw, different tube types, different days, and different subjects. Data shown as methylated molecules (y-axis) for different buffy coats (x-axis).

FIG. 7 shows hierarchical clustering of methylation profiles for buffy coats i.e., 5000 g.e. of buffy coat) isolated from different tubes of the same blood draw, different tube types, different days, and different subjects. Cluster distance was calculated using the L1 norm.

FIG. 8 shows data for pan-cancer assay performance in contrived samples. Data is presented as normalized methylated QE (y-axis) for each contrived samples from different cancer types. Contrived samples were prepared at 0% tumor fraction and 5% tumor fraction.

FIGS. 9A-9F show data for non-limiting examples of loci used in a “personalized blacklist.” Data is presented as Tumor QE normalized (y-axis: “ge_norm”) for each of the 12 samples indicated on the x-axis in cfDNA and buffy coat. FIG. 9A shows tumor QE (normalized) for the intron cg03134157_77. FIG. 9B shows tumor QE (normalized) for the intron cg20907051_11. FIG. 9C shows tumor QE (normalized) for the intron_cg12880300_0. FIG. 9D shows tumor QE (normalized) for the locus IRX4_cg13974394_0. FIG. 9E shows tumor QE (normalized) for the locus EMID2_cg25290307_0. FIG. 9F shows tumor QE (normalized) for intron_cg11453719_0.

FIG. 10 shows normalized total tumor molecules (y-axis) for the specimens on the x-axis based on the global blacklist thresholds described herein (e.g., (1) max tumor QE in any non-cancer specimen >15; and (2) mean tumor QE (including 0s)>2).

FIG. 11 shows normalized total tumor molecules for the new blacklist (y-axis) compared to the normalized total tumor molecules for the old blacklist. Each blacklist was applied to methylation data for the 30 subjects listed in FIG. 11 and plotted in the graph.

FIGS. 12A-12D show data for 2 subjects (out of 30), Subject 8272 and Subject 6885, that had slightly different methylation results after applying the new blacklist. Data is presented as normalized total tumor molecules (y-axis) for day 0, 63, and 154 since first collection. FIG. 12A shows data for Subject 8272 using old blacklist. FIG. 12B shows data for Subject 8272 using new blacklist. FIG. 12C shows data for Subject 6885 using old blacklist. FIG. 12D shows data for Subject 6885 using new blacklist.

FIGS. 13A-13E show data for 5 subjects (i.e., BTO-1 (FIG. 13A), BTO-2 (FIG. 13B), BTO-3 (FIG. 13C), BTO-4 (FIG. 13D), and BTO-5 (FIG. 13E)) generated using the new global blacklist.

FIG. 14 shows Response Scores measured in triplicate at each tumor fraction for each tumor type.

FIG. 15 shows Response Scores measured in triplicate at each tumor fraction for each tumor type.

FIG. 16 shows Response Scores measured for 8 cancer subjects, each with samples from two time points. 8 cancer subjects included three cancer types (lung=4 subjects, pancreas=3 subjects, endometrial=1 subject). Colors indicate the call that was made based on the change in Response Score.

FIG. 17 shows Response Scores measured for 12 healthy subjects, each with samples from two time points. The cfDNA sample at time point 1 for subject HV006 failed sample QC criteria, so no call was made for that subject. No change or Indeterminate calls were made for the remaining 11 subjects.

FIG. 18 shows sensitivity for each tumor fraction plotted with 95% confidence intervals. The dotted line indicates 95% sensitivity, i.e., the limit of detection sensitivity threshold. For each tumor fraction, sensitivity was calculated by comparing each of the 16 replicates to each of the 16 0% tumor fraction replicates (256 comparisons total).

FIG. 19 shows box-plots for the median and interquartile range of Response Scores at each tumor fraction. The standard deviation and mean of each tumor fraction were used to calculate CV.

FIG. 20 shows Simulated Response Scores for 1% and 2% Tumor DNA Samples.

FIG. 21 shows methylation analysis for a 56 year old male subject with pancreatic ductal adenocarcinoma stage IV at time of first collection. Subject was treated with folfirinox (“folfiri”), followed by folfiri maintenance, and followed by folox. FIG. 22 shows tumor QE (y-axis) over days since last treatment (x-axis). Triangles indicate clinician-mediated staging of cancer using imaging.

FIG. 22 shows methylation analysis for a 50 year old male subject with pancreatic adenocarcinoma stage IV at time of first collection. Subject was treated with fluorouracil 5000 mg continuous over 46 hours+leucovorin 800 mg+oxaliplatin 180 mg+Irinotecan 300 mg. FIG. 23 shows tumor QE (y-axis) over days since last treatment (x-axis). Triangle indicates clinician-mediated of cancer using imaging.

FIG. 23 shows methylation analysis for an 87 year old male subject with colorectal adenocarcinoma stage IV at time of first collection. Subject was treated with 6 cycles of fluorouracil 1100 mg+leucovorin 1100 mg+Bevacizumab 500 mg. FIG. 23 shows tumor QE (y-axis) over days since last treatment (x-axis). Triangle indicates clinician-mediated of cancer using imaging.

FIG. 24 shows methylation analysis for a 56 year old female subject with lung squamous cell carcinoma stage IV at time of first collection. Subject was treated with 6 docetaxel. FIG. 25 shows tumor QE (y-axis) over days since last treatment (x-axis). Triangle indicates subject entering into hospice care.

FIG. 25 shows results of a treatment selection assay performed on an aliquot of plasma collected at the same time point as the methylation assay for 40 different samples.

FIG. 26 shows for each sample the maximum variant allele fraction (VAF) (y-axis label: max measured VAF on hybrid capture) versus the normalized total tumor QE (x-axis).

FIG. 26 shows a methylation profile for Subject 6885 measured at various time points over the course of about 294 days. In particular, FIG. 27 shows normalized total tumor molecules (y-axis) separated into loci by color. Methylation profiles were measured at day 0, 63, 145, 166, 229, and 294. Numbers on top of bars indicated the percentage of total methylation for each of the loci at that particular time point.

FIG. 27 shows a methylation profile for Subject 5458 measured at various time points over the course of about 250 days. In particular, FIG. 27 shows normalized total tumor molecules (y-axis) separated into loci by color. Methylation profiles were measured at day 0, 35, 63, 119, 201, and 250. Numbers on top of bars indicated the percentage of total methylation for each of the loci at that particular time point.

FIG. 28 shows the Tumor Methylation Score represented as the normalized sum of methylated molecules at >500 loci that are hypermethylated in circulating tumor DNA (ctDNA) for each of the collection dates. Days since first collection are indicated in parenthesis.

FIG. 29 shows an overview of the Northstar Response® assay for generating a tumor methylation score (TMS), according to one embodiment.

FIG. 30 shows the distribution of all detected VAFS in an example cohort.

FIG. 31 illustrates that the longitudinal association between VAF-based TF estimates and methylome-based TF estimates is patient dependent.

FIG. 32 is a 2D histogram of per-patient R² between TF and either sum(VAF) or max(VAF) for an example cohort.

FIG. 33 is a 2D histogram of per-patient R² between TMS and either sum(VAF) or max(VAF) of the example cohort of FIG. 32.

FIGS. 34A and 34B illustrate that mTF is highly correlated with analytical tumor fraction samples.

FIG. 35 illustrates that serial fold-changes are significantly correlated between max(VAF) vs mTF (a) and sum(VAF) vs TMS (b).

FIGS. 36A and 36B show individual cases comparing mTF, TMS, and sum/max of driver mutation VAFs demonstrate a high degree of clinical, longitudinal correlation.

FIGS. 37A and 37B illustrate that mTF correlates with max(VAF) (a) and TMS correlates with sum(VAF) (b).

FIGS. 38A and 38B illustrate that confirmed CHIP-positive mutations do not correlate with mTF and confirmed CHIP-negative VUs do correlate with mTF.

FIG. 39 illustrates CRC has more sub-VAF mutations.

FIG. 40 illustrates that lower KRAS VAF:mTF ratio was observed only in CRC, and only for G12C and Q61H. KRAS mutation type and samples with multiple KRAS mutations are indicated.

FIG. 41 illustrates the performance of TF estimates across 11 tumor types.

FIGS. 42A and 42B illustrate that CHIP genes do not correlate with TMS.

FIGS. 43A-43C illustrate the differences in methylation between healthy cfDNA and tumor cfDNA.

FIGS. 44A and 44B illustrate that absolute quantification across hundreds of loci enables tumor-naive statistical reconstruction of tumor fraction at sub-molecular limit of detection.

FIGS. 45A-45E illustrate that the vast majority of non-tumor methyl-cfDNA is also found in the buffy coat.

FIGS. 46A-46D illustrate the analytical assessment of tumor fraction from methylated DNA.

FIG. 47 shows a representative example of WBC methylation and zero-truncated poisson fit.

FIG. 48 shows a summary of accuracy of tumor fraction algorithms.

FIGS. 49A-49C illustrate how summarizing absolute counts of methylated DNA within loci blocks further improves tumor fraction accuracy.

FIG. 50 illustrates that tumor fraction measurements are highly stable across different methylation loci.

FIG. 51 illustrates that Methylated Tumor Fraction estimates match variant allele fraction (VAF) obtained from driver and non-driver somatic mutation.

FIG. 52 illustrates the limit of blank for detection of methylated loci.

FIG. 53 illustrates the reproducibility of distribution of methylated loci counts in two patient samples.

FIG. 54 illustrates absolute methylation counts in samples from two patients.

FIG. 55 illustrates that the total methylated cell-free DNA is linear with the contrived tumor fraction.

FIG. 56 illustrates Methylated tumor fraction recovers the contrived mixture across samples.

FIGS. 57A-57B illustrate the Correlation of TF vs VAF is comparable to TMS vs VAF.

FIG. 58 shows the association of methylated TF with SNPs in various genes.

FIG. 59 illustrates the correlation between the ratio of VAF with tumor fraction.

FIGS. 60A-60C illustrate correlation of “total methylation” with TMS.

### DEFINITIONS

“Cell-free DNA” or “cfDNA” refers to extracellular DNA circulating in body fluids such as blood plasma.

“Circulating tumor DNA” or “ctDNA” refers to cfDNA that originates from tumor cells.

“Tumor fraction” or “TF” refers to the proportion of cfDNA that is derived from tumor cells.

“Differentially methylated region” or “DMR” refers to a genomic region that exhibits different patterns of DNA methylation between tumor and normal cells.

“Quantitative Counting Templates” or “QCT” refers to synthetic DNA molecules that incorporate a unique sequence tag to facilitate absolute quantification.

### DETAILED DESCRIPTION

In one aspect, the present disclosure provides a method to quantify DNA methylation in a sample containing DNA. The method can include treating the sample to encode the presence or absence of DNA methylation in the DNA; adding to the sample a set of synthetic molecules, the set of molecules comprising target-associated regions having a nucleotide sequence that matches a target sequence region of an endogenous target molecule and variation regions with a nucleotide sequence that does not match a sequence region of an endogenous target molecule; generating a co-amplification mixture comprising an amplified set of synthetic molecules and an amplified set of at least ten target loci from the DNA; sequencing the co-amplification mixture at a read depth of at least one read sequence per molecule in the sample to generate sequence reads; determining a number of the sequence reads that are methylated sequence reads; quantifying (determining) an absolute number of methylated molecules in the sample based on the number of methylated reads from the sample and a number of reads from the set of synthetic molecules; and optionally, aggregating the number of molecules across at least two loci.

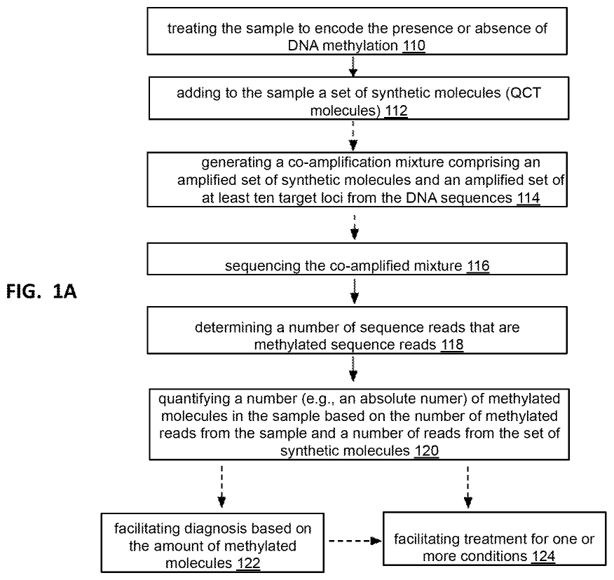

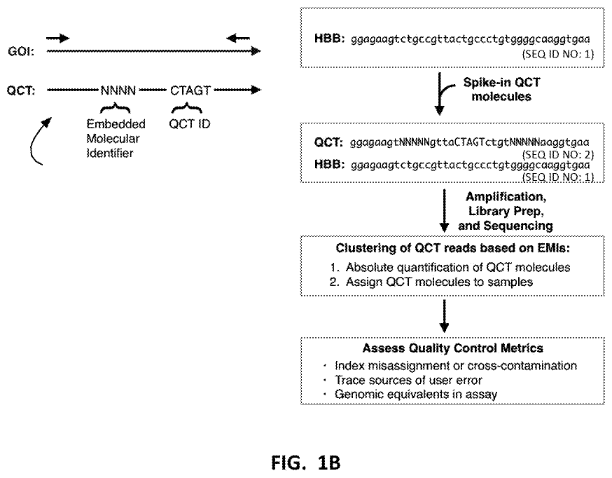

As shown in FIG. 1A, embodiments of a method to quantify DNA methylation in a sample can include: treating the sample to encode the presence or absence of DNA methylation 110; adding to the sample a set of synthetic molecules (e.g., quality control template (QCT) molecules) where each synthetic molecule comprises a target-associated region and a variation region,) 112; generating a co-amplification mixture comprising an amplified set of synthetic molecules and an amplified set of at least ten target loci from the DNA 114; sequencing the co-amplification mixture at a read depth of at least one read sequence per molecule in the sample to generate sequence reads 116; determining a number of the sequence reads that are methylated sequence reads 118; quantifying (determining) a number (e.g., an absolute number) of methylated molecules in the sample based on the number of methylated reads from the sample and a number of reads from the set of synthetic molecules; and facilitating diagnosis based on the amount of methylated molecules 122 and/or facilitating treatment for one or more conditions 124.

In some embodiments, the addition of a spike-in of known sequence and quantity is added to the sample prior to treating the sample to encode the presence or absence of DNA methylation.

In some embodiments, treating the sample to encode presence or absence of DNA methylation is conducted by bisulfite conversion or enzymatic conversion or any other suitable methodology of treatment that separately identifies methylated DNA molecules.

In some embodiments, the set of co-amplification synthetic molecules is a set of quantitative counting templates (QCTs).

In some embodiments, the target loci are chosen due to an increase in DNA methylation in cancerous tissue compared to normal tissue. In some embodiments, the target loci are CpG sites. In some embodiments, the target loci are differentially methylated regions (DMRs). In some embodiments, at least 100 target loci are amplified. In some embodiments, at least 500 loci are amplified. In some embodiments, at least 800 target loci are amplified. In some embodiments, at least 2000 loci are amplified.

In some embodiments, the co-amplification mixture is sequenced at a read depth of at least 1 read per molecule.

In some embodiments, the co-amplification mixture is sequenced at a read depth of 10 or more reads per molecule, 100 or more reads per molecule, or 1000 or more reads per molecule.

In some embodiments, the co-amplification mixture is sequenced at a read depth of at least 1000 sequencing reads per genomic location.

In some embodiments, further comprising aggregating the number of molecules (e.g., counts of methylated DNA) across loci, wherein the loci are cancer-specific loci.

In some embodiments, the counts of methylated DNA may be summed across multiple loci within a haplotype block.

In some embodiments, quantification of DNA methylation is performed in a cell-free DNA sample.

In some embodiments, cell-free DNA from plasma and cell-free DNA from the buffy coat is extracted from the same patient prior to any spike-in step and treating the sample to encode the presence or absence of DNA methylation.

In some embodiments, further comprising aggregating the number of molecules across loci in the cell-free DNA sample, wherein loci that contain lower than a threshold amount of methylated molecules in the buffy coat sample.

In some embodiments, a generated co-amplification mixture comprises an amplified set of spike-in molecules, amplified set of synthetic molecules, and amplified set of at least 10 target loci from the plurality of DNA molecules and at least one locus that demonstrates high methylation in cell-free DNA.

In some embodiments, further comprising aggregating the number of methylated molecules across target loci and at least one locus that demonstrates high methylation in cell-free DNA.

In some embodiments, further comprising normalizing the aggregate number of target methylated molecules by the aggregate number of molecules with high methylation in cell-free DNA.

In some embodiments, further comprising a step of background subtraction.

In some embodiments, further comprising a step of filtering hypermethylated loci.

In some embodiments, further comprising a step of background subtraction and a step of filtering hypermethylated loci.

In some embodiments, the step of background subtraction comprises subtracting the number of methylated molecules (or the methylation signal) as measured in the buffy coat from the number of methylated molecules (or the methylation signal) in the cfDNA.

In some embodiments, further comprising subtracting the number of methylated molecules (or the methylation signal) as measured in the buffy coat from the number of methylated molecules (or the methylation signal) in the cfDNA on a per-locus basis.

In some embodiments, the step of filtering hypermethylated loci comprises filtering target loci with high background methylation prior to quantifying the absolute number of methylated molecules in the sample.

In some embodiments, the filtered target loci include target loci having a number of methylated molecules (or the methylation signal) with buffy coat methylation above a threshold.

In some embodiments, the threshold is sample-specific. In some embodiments, the threshold is subject-specific. In such cases, this is referred to as a “personalized” blacklist. In some embodiments where the threshold is sample-specific or subject-specific the threshold is determined by evaluating the buffy coat methylation signal for loci with high methylation cfDNA signal in non-cancer subjects.

In some embodiments, the threshold comprises a pre-determined mean tumor methylated QE, a predetermined max tumor methylated QE, or a combination thereof.

In some embodiments, the pre-determined mean tumor methylated QE is equal to or greater than 0.5, equal to or greater than 1.0, equal to or greater than 1.5, equal to or greater than 2.0, equal to or greater than 2.5, equal to or greater than 3.0, equal to or greater than 3.0, equal to or greater than 3.5, equal to or greater than 4.0, equal to or greater than 4.5, or equal to or greater than 5.0.

In some embodiments, the pre-determined max tumor methylated QE in any non-cancer specimen is equal to or greater than 5, equal to or greater than 10, equal to or greater than 15, equal to or greater than 20, equal to or greater than 25, equal to or greater than 30, equal to or greater than 35, equal to or greater than 40, equal to or greater than 45, or equal to or greater than 50.

In one embodiment, the threshold comprises (1) max tumor QE in any non-cancer specimen >15; and (2) mean tumor QE (including 0s)>2.

In some embodiments, the target loci are selected to be differentially methylated across at least two cancer tissues of origin.

In some embodiments, at least 5 loci per cancer tissue of origin are targeted.

In some embodiments, cancer tissue of origin is determined based on the abundance of methylated molecules across targeted loci.

An aspect of the present disclosure provides a method to quantify the amount of tumor DNA in a sample containing DNA molecules, the method comprising: i. adding to the sample a set of synthetic molecules, the set of synthetic molecules comprising: target-associated regions having a nucleotide sequence that matches a target sequence region of an endogenous target molecule, and variation regions with a nucleotide sequence that does not match a sequence region of an endogenous target molecule; ii. generating a co-amplification mixture comprising an amplified set of synthetic molecules and an amplified set of at least 10 target loci from the DNA molecules; iii. sequencing the co-amplification mixture at a read depth of at least one read sequence per molecule in the sample to generate sequence reads; iv. quantifying the absolute number of molecules containing a somatic mutation in the sample based on the number of reads from the sample and the number of reads from the set of synthetic molecules; and v. determining a lower bound for variant allele fraction based on the total number of molecules present in the sample at each locus prior to amplification.

An aspect of the present disclosure provides a method to quantify methylation in a DNA sample, the method comprising: adding a spike-in of known sequence to the sample; treating the sample to encode the presence or absence of methylation into the DNA sequence itself, adding to the sample a set of QCT or spike-in molecules, the set of molecules comprising: target-associated regions having a nucleotide sequence that matches a target sequence region of an endogenous target molecule, and variation regions with a nucleotide sequence that does not match a sequence region of an endogenous target molecules; amplifying the mixture of sample and QCT/spike-in molecules at 10 or more different loci simultaneously; sequencing the amplified mixture (at a read depth of at least one read per molecule in the sample); determining the number of reads that are methylated based on the read sequence; computational determining the number of methylated molecules in the sample based on the number of methylated reads from the sample and the number of reads from the QCT/spike-in at each locus; and aggregating the number of molecules across loci.

In some embodiments, the method comprises adding a constant amount of spike-in prior to methylation conversion to each sample so that the efficiency of the conversion process can be monitored.

In some embodiments, treating the sample to encode the presence or absence of methylation into the DNA sequence itself comprises performing a bisulfite conversion or an enzymatic conversion. Bisulfate conversion is described in U.S. Pat. No. 8,257,950, which is incorporated herein by reference in its entirety. Bisulfite conversion and enzymatic conversion converts unmethylated cytosines to uracils. Thus, whether each read came from a methylated molecule or not can be determined by determining the number of reads that are methylated based on the read sequence.

In some embodiments, QCTs can be used to quantify each locus. Alternatively, in other embodiments, spike-in molecules can be used to quantify each locus.

In some embodiments, amplifying the mixture of samples and QCT/spike-in molecules at 10 or more different loci simultaneously. In some embodiments, these locations are chosen based on being more methylated in tumors compared to normal tissue of the same tissue type. In some embodiments, more than 100 locations are amplified. In some embodiments, more than 800 locations are amplified. In some embodiments, these loci have increased methylation in cancer.

In some embodiments, the method includes sequencing the amplified mixture (e.g., at a read depth of at least one read per molecule in the sample). The read depth qualifier could be optional. In some embodiments, the read depth qualifier is at least 1000 reads per locus (e.g., based on the expectation of 1000-2000 sample molecules at each genomic location per tube of blood).

In some embodiments, treating the sample to encode the presence or absence of methylation into the DNA sequence itself comprises bisulfite conversion or an enzymatic conversion.

In some embodiments, the method includes computational determining the number of methylated molecules in the sample based on the number of methylated reads from the sample and the number of reads from the QCT/spike-in at each locus. For spike-ins, the method includes dividing the number of sample reads by the number of spike-in reads times the number of spike-ins added to the sample.

In some embodiments, the method includes aggregating the number of molecules across loci. In certain embodiments, adding up a subset of loci (e.g. adding up just the lung cancer specific loci).

An aspect of the present disclosure includes a method to quantify methylation in a cell-free DNA sample, the method includes extracting cell-free DNA from the plasma and DNA from the buffy coat from the same person; adding a spike-in of known sequence to each DNA sample; treating each DNA sample to encode the presence or absence of methylation into the DNA sequence itself, adding to each DNA sample a set of QCT or spike-in molecules, the set of molecules comprising: target-associated regions having a nucleotide sequence that matches a target sequence region of an endogenous target, and variation regions with a nucleotide sequence that does not match a sequence region of an endogenous target molecules; amplifying the mixture of sample and QCT/spike-in molecules at 10 or more different loci simultaneously; sequencing the amplified mixture (at a read depth of at least one read per molecule in the sample); determining the number of reads that are methylated based on the read sequence; and computational determining the number of methylated molecules in the sample based on the number of reads from the sample and the number of reads from the QCT/spike-in at each locus; and aggregating.

In some embodiments, the method includes aggregating the number of molecules across loci in the cell-free DNA sample while excluding loci with significant amounts of methylated molecules in the buffy coat sample. In order to determine significance, if any, the method includes determining a threshold number of molecules.

For example, cfDNA has background methylation signal that differs from patient to patient. Because the majority of cell-free DNA originates from white blood cells, one aspect of the method can use the methylation signal from the buffy coat as a baseline. In some embodiments, the method includes minimizing the cfDNA background signal, by ignoring any loci with significant methylation in the buffy coat (e.g., useful for minimal residual disease (MRD) detection applications). In other embodiments, the method avoids performing background subtraction by choosing loci that have low methylation background in most people.

An aspect of the present disclosure includes a method to quantify methylation in a DNA sample, the method comprising: adding a spike-in of known sequence to the sample; treating the sample to encode the presence or absence of methylation into the DNA sequence itself, adding to the sample a set of QCT or spike-in molecules, the set of molecules comprising: a) target-associated regions having a nucleotide sequence that matches a target sequence region of an endogenous target, and b) variation regions with a nucleotide sequence that does not match a sequence region of an endogenous target molecule; amplifying the mixture of sample and QCT/spike-in molecules at least 10 different target loci simultaneously and at least one locus known to be highly methylated in cfDNA; sequencing the amplified mixture (at a read depth of at least one read per molecule in the sample); determining the number of reads that are methylated based on the read sequence; computational determining the number of methylated molecules in the sample based on the number of reads from the sample and the number of reads from the QCT/spike-in at each locus aggregating the number of methylated molecules across target loci and highly methylated loci; and normalizing the aggregate number of target methylated molecules by the aggregate number of highly methylated loci. For example, normalizing the aggregate number of target methylated molecules by the aggregate number of highly methylated loci can include 100 methylated molecules for 5000 input molecules.

For example, in some embodiments, the method can monitor the amount of ctDNA over time. Thus, the measurements of methylated molecules may need to be consistent relative to the total input amount of DNA. For example, if a patient's cancer is stable, but 2× the amount of cfDNA is collected in the second time point compared to the first time point, it could appear that the cancer doubled in size even though the cancer remained the same size. In one aspect of the present methods, the present method can look at loci that are highly methylated in cfDNA regardless of whether the cfDNA came from the tumor or not, and normalize the target loci to these highly methylated control loci.

An aspect of the present disclosure includes a method to quantify methylation in a DNA sample, the method comprising: treating the sample to encode the presence or absence of methylation into the DNA sequence itself, amplifying the mixture of sample and QCT/spike-in molecules at 10 or more different target loci simultaneously and at least one locus (e.g. at least one locus, at least two loci, at least three loci, at least four loci, at least five loci, at least six loci, at least seven loci, at least eight loci, at least nine loci, at least ten loci, and the like) known to be highly methylated in cfDNA; sequencing the amplified mixture (at a read depth of at least one read per molecule in the sample); determining the number of reads that are methylated based on the read sequence; computational determining the average number of methylated reads in target loci and the average number of methylated reads in highly methylated loci; aggregating the number of methylated reads across target loci and highly methylated loci; and normalizing the aggregate number of target methylated reads by the aggregate number of highly methylated loci methylated reads.

An aspect of the present disclosure includes a method to quantify methylation in a DNA sample, the method comprising: adding a spike-in of known sequence to the sample; treating the sample to encode the presence or absence of methylation into the DNA sequence itself, adding to the sample a set of QCT or spike-in molecules, the set of molecules comprising: a) target-associated regions having a nucleotide sequence that matches a target sequence region of an endogenous target, and b) variation regions with a nucleotide sequence that does not match a sequence region of an endogenous target molecule; amplifying the mixture of sample and QCT/spike-in molecules at 10 or more different loci simultaneously, with loci chosen to be differentially methylated across at least two cancer tissues of origin; sequencing the amplified mixture (at a read depth of at least one read per molecule in the sample) determining the number of reads that are methylated based on the read sequence; computational determining the number of methylated molecules in the sample based on the number of methylated reads from the sample and the number of reads from the QCT/spike-in at each locus; and determining the tissue of origin based on the abundance of methylated molecules across loci.

For example, methylation patterns are different across tumor tissues of origin. In one aspect of the present methods, the method includes determining the tumor tissue of origin from a cfDNA sample based on which locations have methylated molecules.

In some embodiments, amplifying the mixture of sample and QCT/spike-in molecules at 5 or more loci (e.g., loci per cancer tissue of origin).

In some embodiments, determining the tissue of origin on the abundance of methylated molecules across loci comprises assigning a score to each tissue type.

In an aspect of the present disclosure, provided herein is a method to quantify the amount of tumor DNA in a DNA sample, the method comprising: adding to the sample a set of QCT or spike-in molecules, the set of molecules comprising: a) target-associated regions having a nucleotide sequence that matches a target sequence region of an endogenous target, and b) variation regions with a nucleotide sequence that does not match a sequence region of an endogenous target molecules; amplifying the mixture of sample and QCT/spike-in molecules at 10 or more different loci simultaneously; sequencing the amplified mixture (at a read depth of at least one read per molecule in the sample); computational determining the number of molecules containing a somatic mutation in the sample based on the number of reads from the sample and the number of reads from the QCT/spike-in at each locus; and determining a lower bound for variant allele fraction based on the total number of molecules initially present in the sample at each locus.

In another aspect, this disclosure features a method of determining a DNA methylation profile in a subject, the method comprising: treating a sample isolated from subject to encode the presence or absence of DNA methylation in the DNA molecules; adding to the sample a set of synthetic molecules, the set of molecules comprising: target-associated regions having a nucleotide sequence that matches a target sequence region of an endogenous target, and variation regions with a nucleotide sequence that does not match a sequence region of an endogenous target molecule; generating a co-amplification mixture comprising an amplified set of synthetic molecules and an amplified set of at least ten target loci from the DNA molecules; sequencing the co-amplification mixture at a read depth of at least one read sequence per molecule in the sample to generate sequence reads; determining a number of the sequence reads that are methylated sequence reads; quantifying (determining) a number of methylated molecules for each target loci in the sample based on the number of methylated sequence reads from each target loci in the sample and a number of reads from the set of synthetic molecules; and determining the methylation pattern in the subject based on the number of methylated molecules for target loci in the sample.

In another aspect, this disclosure features a method of determining a DNA methylation profile in a subject over time, the method comprising: at a first time point: i) treating a sample isolated from subject to encode the presence or absence of DNA methylation in the DNA molecules; ii) adding to the sample a set of synthetic molecules, the set of molecules comprising: target-associated regions having a nucleotide sequence that matches a corresponding nucleotide sequence to an endogenous target, and variation regions with a nucleotide sequence that does not match a sequence region of an endogenous target molecule; iii) generating a co-amplification mixture comprising an amplified set of synthetic molecules and an amplified set of at least ten target loci from the DNA molecules; iv) sequencing the co-amplification mixture at a read depth of at least one read sequence per molecule in the sample to generate sequence reads; v) determining a number of the sequence reads that are methylated sequence reads; vi) quantifying (determining) a number of methylated molecules for each target loci in the sample based on the number of methylated sequence reads from each target loci in the sample and a number of reads from the set of synthetic molecules; repeating steps i) to vi) at a second time point; and determining the methylation pattern in the subject based on the number of methylated molecules for each target loci in the sample at the first time point and the number of methylated molecules for each target loci in the sample at the second time point.

In some embodiments of determining a DNA methylation profile in a subject over time, the method includes repeating steps i) to vi) at a third time point, at a fourth time point, or at a fifth time point; and determining the methylation pattern in the subject based on the number of methylated molecules for each target loci in the sample at the third time point, at a fourth time point, or at a fifth time point.

In some embodiments of determining a DNA methylation profile in a subject, the method includes, upon detection of a change in the DNA methylation profile, performing a treatment selection assay on the subject. In some embodiments, the treatment selection assay comprises genomic profiling to detect novel somatic mutations, the abundance of somatic mutations, or both.

In some embodiments, the method detects a methylation signal at each loci, thereby creating a methylation profile at each time point comprising an aggregation of the methylation signals at each loci (see, e.g., Example 10). The methylation profile can then change over time with each time point represented as an aggregation of the methylation signals at each loci (see, e.g., Example 10).

Quantifying methylation profiles over time can enable monitoring of changes in methylation profiles that are indicative of changes in disease progression. For example, a large increase in the percentage that one or more loci contribute to the methylation profile (i.e., a large increase in the composition of the methylation pattern associated with one or more loci) from one point to another time point may indicate an increase in disease progression (e.g., an increase in tumor size). A change in methylation profile can also be indicative of a response to treatment. For example, if the percentage of methylated loci decreases after a treatment for the cancer has been administered, this can indicate a decrease in tumor size and a positive response to the treatment. This allows for informed treatment decisions.

In another aspect, this disclosure features a method for quantifying the abundance of a somatic mutation in a sample containing DNA molecules, the method comprising: determining the abundance of the somatic mutation in the sample; quantifying the DNA methylation in the sample comprising the steps of: treating the sample to encode the presence or absence of DNA methylation at loci of the DNA molecules, wherein the sample comprises at least ten target loci from the DNA molecules; adding to the sample a set of synthetic molecules, the set of molecules comprising: target-associated regions having a nucleotide sequence that matches a target sequence region of an endogenous target, and variation regions with a nucleotide sequence that does not match a sequence region of an endogenous target molecule; generating a co-amplification mixture comprising an amplified set of synthetic molecules and an amplified set of at least ten target loci from the DNA molecules; sequencing the co-amplification mixture at a read depth of at least one read sequence per molecule in the sample to generate sequence reads; determining a number of the sequence reads that are methylated sequence reads; quantifying (determining) an absolute number of methylated molecules in the sample based on the number of methylated reads from the sample and a number of reads from the set of synthetic molecules; and returning a true call result for the abundance of the somatic mutation when the number of methylated molecules is above a predetermined or dynamically calculated threshold; and a no-call result for the abundance of the somatic mutation when the number of methylated molecules is at or below the predetermined or dynamically calculated threshold.

In some embodiments, the somatic mutation is selected from one or more of: a single nucleotide variant, a copy number alteration, an insertion, and a deletion.

In some embodiments, determining the abundance of the somatic mutation comprises using a variant allele fraction (VAF).

In some embodiments, the abundance of the somatic mutation indicates the presence of cancer when the abundance of the somatic mutation is at or above a predetermined threshold.

In some embodiments, the method includes, upon returning of a true call and the abundance of the somatic mutation indicates the presence of cancer, performing a treatment selection assay on the subject. In some embodiments, the treatment selection assay comprises genomic profiling to detect novel somatic mutations, the abundance of somatic mutations, or both.

In some embodiments, the method also includes, upon returning of a no-call, repeating the method of determining abundance of a somatic mutation and quantifying DNA methylation on a different sample taken from the subject.

Embodiments of the methods described herein, for example as shown in FIG. 1A, can be performed in any suitable manner. Embodiments of the method as shown in FIG. 1A can include every combination and permutation of the various system components and the various method processes, including any variants (e.g., embodiments, variations, examples, specific examples, figures, etc.), where portions of embodiments of the method and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method as described in FIG. 1A can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method (e.g., as shown in FIG. 1A), and/or variants without departing from the scope defined in the claims.

### Generating Synthetic Molecules and Adding the Synthetic Molecules to the Sample

In some embodiments, the method to quantify DNA methylation in a sample can include: adding, to the treated sample, a set of synthetic molecules (e.g., QCT molecules), the set of synthetic molecules including: target-associated regions with sequence similarity to a target sequence region of endogenous target molecules (e.g., associated with the genetic disorder; etc.), and variation regions (e.g., including embedded molecular identifier (EMI) regions including a set of variable “N” bases, where each “N” base is selected from any one of an “A” base, a “G” base, a “T” base, and a “C” base) with sequence dissimilarity to a sequence region of the endogenous target molecules.

In some embodiments, the methods can include generating a set of QCT molecules, which can function to generate molecules to be used (e.g., added, processed, sequenced, etc.) at one or more stages (e.g., steps, phases, periods, time periods, etc.) of at least one of sequencing library preparation and sequencing (e.g., high-throughput sequencing, etc.), such as for facilitating downstream computational processing (e.g., determining number of QCT sequence reads for facilitating quantifying the number (or percentage) of methylated molecules).

In some embodiments, synthetic molecules (e.g., QCT molecules) include target-associated regions (e.g., one or more target-associated regions per QCT molecule; etc.). As shown in FIG. 1B, target-associated regions include sequence similarity (e.g., full sequence similarity; sequence similarity satisfying a threshold condition; sequence similarity of a specified number of bases; etc.) to one or more target sequence regions of one or more target molecules (e.g., endogenous target molecules; corresponding to one or more biological targets; etc.), but can additionally or alternatively include any suitable association with any suitable components of one or more target molecules. Target-associated regions preferably enable co-amplification of the corresponding QCT molecules (e.g., including the target-associated regions, etc.) and nucleic acid molecules (e.g., nucleic acids, nucleic acid fragments, etc.) including the target sequence region, which can facilitate improved accuracy in molecular counting (e.g., in determining molecule count parameters; by accounting for amplification biases; etc.), but can additionally or alternatively enable any suitable processes associated with the sequencing library preparation, sequencing, and/or portions of embodiments of the methods described herein. In an example, the methods described herein can include co-amplification of the set of QCT molecules and nucleic acid molecules including the biological target, based on the sequence similarity of the target-associated region and the target sequence region of the biological target, and where quantifying the DNA methylation in the sample can include determining a target molecule count (e.g., methylated molecule count) describing a number of methylated molecules associated with the sequencing, based on the set of synthetic molecules (e.g., QCT sequence read clusters).

In some embodiments, synthetic molecules (e.g., QCT molecules) can omit target-associated regions. For example, QCT molecules can be used with components of samples including biological targets, without target-association (e.g., without having pre-determined similarity to target sequence regions of the biological targets) and/or without corresponding co-amplification with components of the samples (e.g., nucleic acid molecules including the target sequence regions; etc.). In some examples, QCT molecules can be pre-processed to be adapted to sequencing, such as where the pre-processed QCT molecules can be added to a processed sample suitable for sequencing, to be co-sequenced without the need for co-amplification (e.g., for improving user friendliness). QCT molecules omitting target-associated regions are preferably usable for facilitating contamination parameter determination but can additionally or alternatively be used for facilitating any suitable sequencing-related parameter determination. In a specific example, the set of QCT molecules can be adapted for subsequent sequencing (e.g., high-throughput sequencing such as NGS; etc.), where generating the set of QCT molecules can include amplifying a first subset of QCT molecules (e.g., each including a first shared QCT identifier region; etc.) of the set of QCT molecules; and amplifying a second subset of QCT molecules (e.g., each including a second shared QCT identifier region; etc.) of the set of QCT molecules, where the QCT molecule sequencing reads are derived from the sequencing corresponding to: a QCT mixture generated based on the first subset of QCT molecules and the sample including the biological target (e.g., including first target molecules corresponding to the biological target; etc.), and an additional QCT mixture generated based on the second subset of QCT molecules and an additional sample including the biological target (e.g., including second target molecules corresponding to the biological target; etc.), where the sample and the additional sample respectively correspond to a first sample compartment and a second sample compartment of the sample compartments. However, target-associated regions and/or QCT molecules omitting target-associated regions can be configured in any suitable manner.

In some embodiments, synthetic molecules (e.g., QCT molecules) include one or more variation regions (e.g., one or more variation regions per QCT molecule; adjacent variation regions; separated variation regions; etc.). As shown in FIG. 1B, a variation region preferably includes sequence dissimilarity (e.g., complete sequence dissimilarity; dissimilarity of a specified number of bases; partial sequence dissimilarity; etc.) to one or more sequence regions (e.g., distinct from a target sequence region; etc.) of target molecules. A variation region can additionally or alternatively include one or more EMI regions. In a variation, an EMI region can include a set of variable “N” bases (e.g., one or more variable “N” bases, etc.), where each “N” base is selected (e.g., randomly selected; selected according to predetermined statistical distributions and/or probabilities; etc.) from any one of an “A” base, a “G” base, a “T” base, and a “C” base. In a variation, an EMI region can include a synthesized region (e.g., on a microarray; using silicon-based synthesis; etc.) including one or more specified bases (e.g., designed and synthesized bases; etc.), such as synthesized regions designed to facilitate QCT sequence read cluster determination (e.g., by maximizing pairwise hamming distance between EMI regions; etc.). In variations, a QCT molecule can additionally or alternatively include a plurality of EMI regions (e.g., a variation region including a plurality of EMI regions; adjacent EMI regions; separated EMI regions; EMI regions including variable “N” bases; EMI regions including synthesized regions; etc.). For example, each variation region of the set of QCT molecules can include an embedded molecular identifier region including a set of variable “N” bases, where each “N” base is selected from any one of an “A” base, a “G” base, a “T” base, and a “C” base, where each QCT molecule of the set of QCT molecules further includes an additional EMI region including an additional set of variable “N” bases, where the additional EMI region is separated from the EMI region by a sequence region of the QCT molecule, such as where the set of variable “N” bases and the additional set of variable “N” bases can each include a determined (e.g., predetermined) number of “N” bases (e.g., greater than three “N” bases, greater than any suitable number of “N” bases, an exact number of “N” bases; etc.), and where determining a sequencing-related parameter (e.g., contamination parameter) can be based on QCT sequence read clusters derived based on the EMI regions and the additional EMI regions of the set of QCT molecules (e.g., based on distinct EMI sequence reads corresponding to pairs of an EMI region and an additional EMI region; etc.).

In some embodiments, as shown in FIG. 1B, a QCT molecule can include a QCT identifier region identifying the QCT molecule (and/or other suitable QCT molecules), such as a shared QCT identifier region (e.g., a shared sequence region, with dissimilarity to one or more sequence regions of the target molecules, etc.) identifying QCT molecules belong to a set of QCT molecules (e.g., where different QCT identifier regions are unique to different sets of QCT molecules, etc.). In an example, the variation region of the each QCT molecule of a first set of QCT molecules can include a first EMI region separated from a second EMI region by at least a first QCT identifier region, where each additional QCT molecule of a second set of QCT molecules can include a first additional EMI region separated from a second additional EMI region by at least a second QCT identifier region. In an example, the first, the second, the first additional, and the second additional EMI regions can include a set of variable “N” bases, and where each “N” base is selected from any one of an “A” base, a “G” base, a “T” base, and a “C” base, and where computationally determining the set of QCT sequence read clusters can include determining the set of QCT sequence read clusters based on the first and the second QCT identifier regions, and on the first, the second, the first additional, and the second additional EMI regions. In an example, for the each QCT molecule of the first set of QCT molecules, the corresponding QCT molecule sequence is characterized by full sequence similarity to a first sequence template of the biological target except for the first QCT identifier region, the first EMI region, and the second EMI region; and where, for the each additional QCT molecule of the second set of QCT molecules, the corresponding additional QCT molecule sequence is characterized by full sequence similarity to a second sequence template except for the second QCT identifier region, the first additional EMI region, and the second additional EMI region. In a specific example, QCT molecule sequences can be identical to the target molecule sequence (e.g., one or more regions of the target molecule sequence; etc.), except for two separate sections of 5N sequences interrupted by a distinct, previously determined QCT identifier region (e.g., unique identifier sequence, etc.). In a specific example, QCT identifier regions (e.g., unique QCT ID sequence, as shown in FIG. 1i), can be used to enable the use of multiple QCT libraries that can be added at one stage for internal control or at different stages for tracking of loss of input biological targets or other user errors. Additionally or alternatively, QCT identifier regions can be configured in any suitable manner. However, QCT molecules can include any suitable combination of any suitable type of regions (e.g., where different QCT molecules include the same or different types of and/or number of regions; with any suitable sequence similarity and/or dissimilarity to sequence regions of target molecules; etc.).

In some embodiments, the method can additionally or alternatively include generating one or more QCT libraries (e.g., each QCT library including QCT molecules, etc.) such as where a QCT library can include multiple sets of QCT molecules such as where each set of QCT molecules is identifiable by a different QCT identifier region. In an example, generating a QCT library can include amplifying different sets of QCT molecules (e.g., for preparation for sequencing, such as where the QCT molecules are amplified prior to addition to one or more components of a sample to generate a QCT mixture; etc.). In examples, generating a QCT library can include determining a number of QCT molecules to include in the QCT library. In a specific example, the solutions to the birthday problem can be used to determine the maximum number of unique QCT molecules that should be included in each sample given a particular diversity of QCT molecules, such as where, for 4\hat{ }10 sequences, which can be generated by 10 variable N bases in a QCT molecule, up to 1200 QCT molecules can be used with probability of ~0.5 of a single valid EMI collision (exp(−1200*1199/2/4\hat{ }10)~0.5), and where at 200 QCT molecules, the probability of a single valid collision is ~2%. In a specific example, generating a QCT library can include generating a QCT library adapted for deployment (e.g., at a single stage of the at least one of the sequencing library preparation and the high throughput sequencing, etc.) of less than 0.00001 nanograms (and/or other suitable amounts) of amplifiable QCT molecules for each sample of a set of samples. However, determining the number of QCT molecules to include in a QCT library, and generating QCT libraries, can be performed in any suitable manner.

In one embodiment, the QCT libraries can be generated by synthesizing complementary strand to single-stranded oligonucleotide sequences that contain variable “N” sequences. In a specific example, double stranded QCT libraries can be generated by re-suspending and annealing the QCT ultramers with a complementary primer sequence, extending the sequences using Klenow Fragment (exo-), and treating with Exonuclease I. The final product can be purified to remove unused single stranded DNA molecules, and QCT libraries can be quantified using fluorometric assays such as Qubit HS assay, from which the number of QCT molecules to be added to each sample can be calculated by using the expected molecular weight of the double-stranded QCT molecules. However, generating QCT molecules can be performed in any suitable manner.

In some embodiments, synthetic molecule libraries (e.g., QCT libraries) can be added at different sequencing library preparation stages (e.g., sample preparation stages) and/or sequencing stages to trace loss-of-sample. In one embodiment, if a first set of QCT molecules (e.g., QCT1 molecules; first QCT molecules including a first shared QCT identifier region; etc.) is dispensed at the point of sample collection, and an equal amount of a second set of QCT molecules (e.g., QCT2 molecules; second QCT molecules including a second shared QCT identifier region; etc.) is dispensed after sample purification (or after treating the sample to encode the presence or absence of DNA methylation), the purification yield (or treatment efficiency yield) may be assessed via comparisons of molecules counts for the first set of QCT molecules and the second set of QCT molecules (e.g., QCT1 vs QCT2 molecule counts, etc.). In one example, the synthetic molecule libraries can be used to trace the efficiency of treating the sample to encode the presence or absence of DNA methylation (i.e., measure bisulfite conversion efficiency).

In some embodiments, the set of synthetic molecules includes: target-associated regions having a nucleotide sequence that matches a target sequence region of an endogenous target, and variation regions with a nucleotide sequence that does not match a target sequence region of an endogenous target molecule. As used herein, the term “matches” refers to sufficient complementarity between two nucleotide sequences where the sequences bind and form the basis for a nucleic acid extension reaction (e.g., amplification). Sufficient complementarity can refer to a match with up to 5 mismatches between the two nucleotide sequences (e.g., the target associated region on a synthetic molecule and the target sequence region on the endogenous target). As used herein, the phrase “does not match” refers to insufficient complementarity between two nucleotide sequences such that sufficient Watson-Crick base pairing is not achieved to enable the two nucleotide sequences to bind and no or little amplification occurs when put into an amplification reaction. Insufficient complementarity can refer to a match with greater than 5 mismatches between the two nucleotide sequences (e.g., the target associated region on a synthetic molecule and the target sequence region on the endogenous target).

### Co-Amplification and Sequencing the Co-Amplification Mixture

In some embodiments, generating a co-amplification mixture comprising an amplified set of synthetic molecules and an amplified set of at least ten target loci from the DNA molecules comprises includes amplifying a synthetic molecule (e.g., QCT molecule) comprising a target-associated region and a nucleic acid molecule (e.g., a nucleic acid that encodes the presence or absence of DNA methylation). For example, target-associated regions preferably enable co-amplification of the corresponding QCT molecules (e.g., including the target-associated regions, etc.) and nucleic acid molecules (e.g., nucleic acids, nucleic acid fragments, etc.) including the target sequence region, which can facilitate improved accuracy in molecular counting (e.g., in determining molecule count parameters; by accounting for amplification biases; etc.), but can additionally or alternatively enable any suitable processes associated with the sequencing library preparation, sequencing, and/or portions of embodiments of the method. In an example, co-amplification of the set of QCT molecules and nucleic acid molecules including the methylated DNA is based on the sequence similarity of the target-associated region and the target sequence region of the methylated DNA.

In some embodiments, the sample includes DNA molecules with loci that are expected to have an increase in DNA methylation in cancerous tissue compared to non-cancerous tissue. Loci that are expected to have an increase in DNA methylation in cancerous tissue compared to non-cancerous tissue are chosen based on, for example and without limitation, a population based survey of methylated loci a patient specific metric (e.g., methylation patterns from a tumor), and/or literature-based studies that identify methylated loci.

In some embodiments, the loci that have an expected increase in DNA methylation in cancerous tissue compared to non-cancerous tissue are amplified and analyzed for the presence of DNA methylation. In some embodiments, the loci that have an expected increase in DNA methylation in cancerous tissue compared to non-cancerous tissue include at least 10, 20, 50, 100, 200, 300, 400, 500, 600, 700, 800, 900, 1000 or more locus, where each locus is expected to have an increase in DNA methylation in cancerous tissue compared to non-cancerous tissue. In some embodiments, the loci that have an expected increase in DNA methylation in cancerous tissue compared to non-cancerous tissue also include loci (e.g., normalization loci) where the DNA sequence is methylated (e.g., highly methylated) in both cancerous tissue and non-cancerous tissue. In such cases, generating a co-amplification mixture results in the co-amplification mixture including: an amplified set of synthetic molecules, an amplified set of a set (e.g., at least ten target loci that have an expected increase in DNA methylation in cancerous tissue compared to non-cancerous tissue), and an amplified set of at least one normalization loci (e.g., a loci that is methylated (e.g., highly methylated) in both cancerous and non-cancerous tissue.

In some embodiments, sequencing (e.g., in relation to 116) associated with one or more embodiments of the method 100 preferably includes high throughput sequencing, which can include and/or be associated with any one or more of: NGS, NGS-associated technologies, massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, any generation number of sequencing technologies (e.g., second-generation sequencing technologies, third-generation sequencing technologies, fourth-generation sequencing technologies, etc.), amplicon-associated sequencing (e.g., targeted amplicon sequencing), metagenome-associated sequencing, sequencing-by-synthesis, tunneling currents sequencing, sequencing by hybridization, mass spectrometry sequencing, microscopy-based techniques, and/or any suitable technologies related to high throughput sequencing. In some embodiments, sequencing can include any suitable sequencing technologies (e.g., Sanger sequencing, capillary sequencing, etc.).

### Determining Methylated Molecules and Quantifying Methylated Molecule

In some embodiments, determining the number of methylated molecules in the sample based on the number of methylated reads from the sample and the number of reads from the QCT/spike-in at each locus include one or more of target molecule counts (e.g., absolute molecule count of methylated molecules, such as in the original sample; absolute count of endogenous target molecules, such as in the original sample; etc.); reference molecule counts (e.g., absolute count of endogenous reference molecules; such as in the original sample; etc.); QCT molecule counts (e.g., corresponding to a number of valid QCT sequence read clusters; corresponding to a number of distinct QCT molecules added to components of the sample; etc.); associated ratios (e.g., correction factors; ratios between a molecule count and an associated number of sequence reads; etc.); and/or any other suitable parameters associated with methylated molecule counts.

As show in FIG. 1A, methylated molecule counts are preferably used in facilitating one or more diagnoses 122, but can additionally or alternatively be used for (e.g., as inputs for) any suitable portions of embodiments of the method 100, including facilitating treatment of one or more conditions 124.

In some embodiments, determining a methylated molecule count parameter (e.g., methylated molecule count; etc.) can be based on a correction factor ratio determined based on a QCT molecule count (e.g., corresponding to a number of QCT sequence read clusters, such as a number of valid QCT sequence read clusters; etc.) and QCT molecule sequence reads (e.g., a number of the QCT molecule sequence reads corresponding to the QCT sequence read clusters; etc.), such as by multiplying the number of methylated molecule sequence reads by the correction factor ratio. In a specific example, the number of valid non-contaminating QCT sequence read clusters (e.g., remaining QCT sequence read clusters after discarding the QCT sequence read clusters with 2 or fewer reads, and/or with any suitable number or fewer of reads; etc.) can indicate the QCT molecule count (e.g., the number of QCT molecules for a particular sample compartment; for a particular sample; for a particular sample identifier; etc.). In a specific example, by dividing the QCT molecule count by the sequencing reads resulting from the corresponding QCT molecules, the correction factor can be found, such as where the correction factor multiplied by the sequencing reads belonging to the target molecules (e.g., in the particular sample compartment; from the particular sample; associated with the particular sample identifier; etc.) would result a target molecule count (e.g., an absolute number of initial biological target molecules that were accessible by the assay for amplification; etc.). In an example, the average QCT sequencing depth used in determining the absolute count of the endogenous target molecules and the absolute count of endogenous reference molecules is determined separately from their corresponding QCTs.

Alternatively, in one embodiment, the read depth threshold for discarding QCT sequence read clusters (e.g., for determining molecule count parameters and/or suitable sequencing-related parameters; etc.) can be determined adaptively based on features of QCT molecule sequence read (e.g., EMI sequence read) depth distribution. For example, a threshold may be set for each indexed sample by computing the mean EMI read depth within each sample, computing the square-root of this mean read depth, and discarding QCT sequence read clusters with read depth below the square-root of the mean read depth. Additionally or alternatively, read depth thresholds for discarding QCT sequence read clusters can be computed in any suitable manner. However, determining methylated molecule count can be performed in any suitable manner.

In some embodiments, variant calling comprises determining the variant allele frequency (VAF) of the DNA variant. As used herein, “variant allele frequency” or “variant allele fraction” refers to the percentage of sequence reads observed matching a specific DNA variant divided by the overall coverage at that locus.

In some embodiments (e.g., of quantifying DNA methylation), the method in FIG. 1A can be used a) to determine parameters for use in algorithms for determining the diagnostic outcome of assays, b) to track the loss of input methylated DNA (e.g., at different stages of the assay), c) to return a no-call result when the number of methylated molecules is too low (e.g., to determine when an assay is not reliable, etc.), d) to return a no-call result when a corresponding quantification of an abundance of somatic mutation(s) in a sample is too low (e.g., to determine when an assay is not reliable), e) to design assays for detecting changes in methylation profiles over, and/or f) aiding therapeutic and clinical decision-making based on the results of diagnostic assays.

Embodiments can additionally or alternatively determine the portion of biological material that is accessible by the assay, such as through quantification of the biological targets (e.g., methylated molecules) based on using the QCT molecules, which can improve upon measuring the total genomic material available and calculating the expected biological target (e.g., methylated molecules) concentration, due to not all targets being accessible by assays. In a specific example, this may be due to shearing of DNA to a short size distribution, as in the case of circulating free DNA that is assayed in applications of liquid biopsy applications where circulating tumor DNA is assayed.

Some embodiments of the methods provided herein (see, e.g., FIG. 1A) can be used in association with one or more conditions (e.g., in association with characterizing, diagnosing, treating, and/or performing processes related to one or more conditions; etc.), where the conditions can include and/or otherwise be associated with cancer (e.g., through analyses associated with any suitable oncogenes, cancer biomarkers, and/or other cancer-associated targets; through analyses associated with liquid biopsies), and/or any other suitable conditions. In an example, the method (of FIG. 1A and/or as described herein) can include determining a methylated molecule count (e.g., corresponding to a number of methylated molecules in a sample; based on use of QCT molecules; etc.) for facilitating diagnosis associated with liquid biopsies.

In some embodiments, the number of methylated molecules for the cfDNA and the number of methylated molecules for gDNA are quantified in the same workflow. For example, methylated molecules of cfDNA and methylated molecules of gDNA can be indexed (e.g., using defined index sequences) in order to be able to differentiate between the source of methylated molecules (e.g., cfDNA versus gDNA). This enables the cfDNA and gDNA to be amplified and/or sequenced in the same reaction (e.g., multiplexing).

### Processing the Methylated Sequencing Reads

Some embodiments of the methods provided herein (see, e.g., FIG. 1A) include a step of processing the methylated sequence reads. In some embodiments, processing of the methylated sequence reads includes filtering out selected hypermethylated target loci; and/or subtracting background methylation. The processing step (e.g., filtering out selected hypermethylated target loci and/or subtracting background methylation) can be performed prior to determining the number of methylated molecules in the sample based on the number of methylated reads from the sample and a number of sequence reads from the set of synthetic molecules. The processing step (e.g., filtering out selected hypermethylated target loci and/or subtracting background methylation) can be performed prior to determining the number of molecules in the number of methylated molecules in the sample based on the number of methylated reads from the sample and a number of sequence reads from the set of synthetic molecules but prior to an aggregating step.

In some embodiments, the step of subtracting background methylation includes subtracting the number of methylated molecules as measured in the buffy coat (i.e., gDNA) from the number of methylated molecules in the cfDNA. In such cases, methylated molecules are quantified for both cfDNA and gDNA. For example, methylation measured in gDNA is then subtracted from cfDNA methylation in order to remove background from the cfDNA methylation signal. In some embodiments, the step of background subtraction includes subtracting the number of methylated molecules as measured in the buffy coat (i.e., gDNA) from the number of methylated molecules in the cfDNA on a per-locus basis. For example, methylation measured in gDNA for a particular target loci is subtracted from cfDNA methylation measured for the same target loci in order to remove background from the cfDNA methylation signal on a per-locus basis.

In some embodiments, the step of filtering out selected hypermethylated target loci comprises filtering out target loci having a total number of methylated molecules in the buffy coat above a threshold (e.g., a predetermined number of quantified methylated molecules). In some embodiments, the step of filtering out selected hypermethylated target loci can include filtering out target loci with high background methylation prior to quantifying the number of methylated molecules in the sample. In some embodiments, the selected hypermethylated target loci that are filtered out include target loci having a high methylation cfDNA signal in non-cancer subjects. In some embodiments, the selected hypermethylated target loci are selected from a global blacklist (see, e.g., Example 6). In some embodiments, the selected hypermethylated target loci are selected from a personal blacklist (see, e.g., Example 6).

In some embodiments, the selected hypermethylated target loci that are filtered out include target loci having a number (e.g., a total number) of methylated molecules in the buffy coat above a threshold. In some embodiments, the threshold is sample specific (e.g., the number of methylated molecules in the buffy coat is determined for each sample). In some embodiments, the threshold is subject-specific (e.g., the number of methylated molecules in the buffy coat is determined for each subject). In some embodiments, the threshold includes a pre-determined mean tumor methylated quantitative equivalent (QE), a predetermined max tumor methylated QE, or a combination thereof. Quantitative equivalents (QE) is an estimate of the number of genomic equivalents of a locus based on QCT analysis. In some embodiments, the threshold includes a max tumor QE in any non-cancer specimen of >15, where a hypermethylated target loci having a max tumor QE of greater than 15 in any non-cancer specimen is filtered out. In some embodiments, the threshold includes a mean tumor QE (including 0s)>2, where a hypermethylated target loci having a mean tumor QE>2 is filtered out.

### Aggregating and Normalizing

Some embodiments of the methods provided herein (see, e.g., FIG. 1A) include a step of aggregating the number of methylated molecules across all or a subset of the target loci (e.g., a subset of the at least ten target loci) to quantify DNA methylation in a sample. In some embodiments, aggregating comprises aggregating the number of methylated molecules across at least two target loci (e.g., at least two of the at least ten target loci) to quantify the DNA methylation in the sample. In some embodiments, the method includes aggregating the methylated molecules in the cfDNA (plasma) sample across at least two loci (e.g., at least two of the at least ten target loci). In some embodiments, the method includes aggregating the methylated molecules in the buffy coat (gDNA) sample across at least two loci (e.g., at least two of the at least ten target loci).

In some embodiments, aggregating the number of methylated molecules across all or a subset of the target loci (e.g., all or a subset of the at least ten target loci) comprises aggregating target loci that contain lower than a threshold amount of methylated molecules in the buffy coat sample. The threshold of amount of methylated molecules in the buffy coat sample can be predetermined or dynamically calculated. A dynamically calculated threshold is a threshold that is not fixed but varies based on certain conditions or inputs. In the case of a threshold amount of methylated molecules, the threshold can be dynamically calculated based on factors such as the size of the system being studied, the concentration of molecules present, or the sensitivity of the detection method used to measure the molecules. For example, in a sample (e.g., a blood draw) the threshold amount of methylated molecules may vary depending on cancer type or the stage of the cancer.

Some embodiments of the methods provided herein (see, e.g., FIG. 1A) include a step of normalizing the aggregate number of methylated molecules to help quantify the DNA methylation in a sample. In one embodiment, the method includes normalizing the aggregate number of target methylated molecules by the methylated molecules in the normalization loci (e.g., methylated molecules for at least one normalization loci). In some embodiments, the method includes normalizing the aggregate number of target methylated molecules by the aggregate number of methylated molecules in the normalization loci. In such cases, the method includes aggregating the number of methylated molecules across at least two normalization loci. In some embodiments, the method includes normalizing the aggregate number of methylated molecules from at least two of the at least 10 target loci by the methylated molecules for the at least one normalization locus.

The aggregating and/or normalizing steps enable quantification of a tumor methylation score that represents the normalized sum of methylated molecules (e.g., at least ten target loci) that are hypermethylated in the sample. The disclosure provides for methods of quantifying DNA methylation with a lower limit of detection of about 0.1 molecules per locus. Counts from multiple loci can be aggregated to achieve an average count for an aggregated group of loci in the detectable range. For example, counts from 4 loci with an average count of 0.03 molecules per locus can be combined to provide a count of 0.12 molecules for the aggregated group of loci, within the limit of detection.

Some embodiments of the methods provided herein (see, e.g., FIG. 1A) can be used for quantifying the abundance of tumor DNA in a sample.

Some embodiments of the methods provided herein (see, e.g., FIG. 1A) can be used for determining DNA methylation profile in a subject.

Some of the embodiments of the methods provided herein (see, e.g., FIG. 1A) can be used for quantifying genetic modifications in a sample. Some embodiments of the methods provided herein (see, e.g., FIG. 1A) can be used for quantifying the abundance of a somatic mutation in a sample. Some embodiments of the methods provided herein (see, e.g., FIG. 1A) can be used for quantifying the abundance of a copy number variation in a sample.

Additionally or alternatively, data described herein (e.g., sequencing-related parameters, identifiers, read depths, sequence reads, sequence region determinations, QCT molecule designs, primer designs, etc.) can be associated with any suitable temporal indicators (e.g., seconds, minutes, hours, days, weeks, time periods, time points, timestamps, etc.) including one or more: temporal indicators indicating when the data was collected, determined, transmitted, received, and/or otherwise processed; temporal indicators providing context to content described by the data, such as temporal indicators indicating the sequence of stages of sequencing library preparation and/or sequencing; changes in temporal indicators (e.g., data over time; change in data; data patterns; data trends; data extrapolation and/or other prediction; etc.); and/or any other suitable indicators related to time.

Additionally or alternatively, parameters, metrics, inputs, outputs, and/or other suitable data described herein can be associated with value types including any one or more of: scores, binary values, classifications, confidence levels, identifiers (e.g., sample identifiers, QCT molecule identifiers, etc.), values along a spectrum, and/or any other suitable types of values. Any suitable types of data described herein can be used as inputs, generated as outputs, and/or manipulated in any suitable manner for any suitable components associated with embodiments of the method 100 and/or system 200.

In some cases the embodiments as described in FIG. 1B can additionally or alternatively include a sample handling network configured to generate molecules (e.g., QCT molecules; QCT libraries; etc.), process biological samples, and/or perform other suitable processes; a sequencing system configured to sequence processed genetic material from mixtures generated based on biological samples and QCT molecules; a computing system (e.g., a remote computing system; a local computing system; etc.) configured to analyze the sequence reads, determine QCT sequence read clusters, determine sequencing-related parameters, facilitate diagnoses, facilitate treatment, and/or perform other suitable processes (e.g., computational processes); and/or any other suitable components. The components of the system in FIG. 1B can be physically and/or logically integrated in any manner (e.g., with any suitable distributions of functionality across the components, such as in relation to portions of embodiments of the method in FIG. 1A; etc.). However, the method FIG. 1A and system FIG. 1B can be configured in any suitable manner.

### Facilitating Diagnosis, Treatment, or Additional Assessment

In one aspect, this disclosure features a method (FIG. 1A) that can additionally or alternatively include facilitating diagnosis 122, which can function to aid, determine, provide, and/or otherwise facilitate one or more diagnoses for one or more conditions.

Facilitating one or more diagnoses can include any one or more of determining one or more diagnoses (e.g., based on number of methylated molecules; etc.); providing one or more diagnoses (e.g., to one or more users; to one or more care providers, such as for use by one or more care providers in providing medical diagnoses to patients; etc.); aiding one or more diagnoses (e.g., providing one or more sequencing-related parameters and/or other suitable parameters to one or more care providers and/or other suitable entities, for use in determining a diagnosis, such as in combination with other data; etc.); and/or any suitable processes associated with diagnoses. For example, aiding diagnosis can include providing a quantification of DNA methylation in a sample from a patient (e.g., to a user; to a care provider; etc.) adapted for use in determination of a diagnostic outcome for assays associated with liquid biopsies. In an example, quantifying DNA methylation can include quantifying the number of methylated molecules in the sample, (e.g., the absolute number of methylated molecules in the sample) for facilitating diagnosis associated with liquid biopsies.

In some embodiments, facilitating diagnosis (e.g., cancer diagnosis) can include facilitating diagnosis based on the amount of methylated molecules in the sample (see FIG. 1A, 122). For example, determining the absolute number of methylated molecules can include determining the absolute number of methylated molecules associated with a DNA sequence, based on dividing the total read count for the methylated molecules by the average QCT sequencing depth, where determining the absolute number of methylated molecules can include determining the absolute number of methylated molecules associated with a second DNA sequence not expected to have a methylation, based on dividing the total read count for the methylated molecules by the average QCT sequencing depth, and where facilitating the diagnosis (e.g., the cancer diagnosis) can include facilitating the diagnosis (e.g., cancer diagnosis; etc.) of the methylated molecule based on the comparison.

The methods provided by this disclosure have increased sensitivity compared to other methods known in the art and therefore allow for the detection of lower amounts of ctDNA. This can make the methods useful for the detection minimal residual disease (MRD), which is the amount of cancer cells that remain after treatment. MRD can be an indicator of likely relapse and is important in guiding treatment decisions.

In some embodiments, facilitating diagnosis (e.g., cancer diagnosis) can include facilitating diagnosis based on the amount of methylated molecules in the sample (see FIG. 1A, 122). For example, determining the absolute number of methylated molecules can include determining the absolute number of methylated molecules, based on dividing the total read count for the methylated molecules by the average QCT sequencing depth, where determining the absolute count of the methylated molecules can include determining the absolute count of the methylated molecules in a sample not expected to have methylated sequence at the same correspond DNA sequence, based on dividing the total read count for the methylated molecules by the average QCT sequencing depth, and where facilitating the diagnosis (e.g., cancer diagnosis) can include facilitating the diagnosis (e.g., cancer diagnosis) of the methylated molecules based on the comparison.

In one aspect, this disclosure features a method (FIG. 1A) that can additionally or alternatively include facilitating treatment 124. For example, the methods described herein, including: the quantification of DNA methylation, the determination of a DNA methylation profile, and/or the quantification of the abundance of a somatic mutation can be used to assess whether a cancer treatment has been effective for a particular patient. The results provided by the methods described herein can help inform an oncologist's future treatment decisions. In one example, where a method to quantify DNA methylation in a sample results in the discovery of increase DNA methylation in a subject, the subject's oncologist can use the quantified DNA methylation increase as a basis, in part, for adjusting the subject's treatment regimen. In another example, where a method to determine the methylation profile in a sample results in the discovery of a change in the DNA methylation profile indicative of a change in the subject's cancer, the subject's oncologist can use the change in DNA methylation profile as a basis, in part, for adjusting the subject's treatment regimen. In another example, where a method for quantifying the abundance of a somatic mutation in a sample results in the discovery of somatic mutations indicative of cancer and/or a change in the cancer, the subject's oncologist can use the quantification of the abundance of the somatic mutation as a basis, in part, for adjusting the subject's treatment regimen. In another example, where a method to quantify DNA methylation in a sample results in the discovery of decreased DNA methylation in a subject, the subject's oncologist can use the quantified DNA methylation increase as a basis, in part, for adjusting the subject's treatment regimen, e.g., to lower the treatment dose or the frequency of administration, or to stop treatment altogether.

A tumor comprises different subclones that each have their own associated set of methylated loci. While undergoing therapy (e.g., chemotherapy), the treatment kills off some but not all subclones, allowing the resistant subclones to proliferate. Eventually, the tumor's composition becomes primarily these resistant subclones. Often, the set of loci that uniquely identify each subclone is not known a priori. This disclosure provides a tumor-naive approach of measuring tumor fraction that can be used to identify that a new cancer subclone has taken over the tumor.

This approach comprises the following steps: (1) Taking a blood sample prior to administration of a therapy; (2) identifying a first set of non-zero loci (set A); (3) identifying a second set of zero loci (set B); (4) taking a blood sample after the first administration of a therapy; (5) determining the tumor fraction on the set A loci; (6) determining the tumor fraction on the set B loci; and (7) performing statistical analysis on the tumor fraction from A and B. If the B tumor fraction is statistically significantly higher than the A tumor fraction, then this indicates that the difference is not due to stochastic noise and that the tumor methylation profile has altered, which suggests that the patient is at risk for developing therapy resistance.

In one embodiment, this disclosure features a method that can additionally or alternatively include recommending additional assessment of the subject based on, which can function to aid, determine, provide, and/or otherwise facilitate diagnosis or treatment. For example, the methods described herein, including: the quantification of DNA methylation, the determination of a DNA methylation profile, and/or the quantification of the abundance of a somatic mutation can be used as the basis for making additional assessments of the subject or recommending that additional assessments of the subject. In some cases, making an additional assessment includes subjecting the subject to a treatment selection assay. In some cases, making an additional assessment includes subjecting the subject to a genomic profiling assay (i.e., an assay to assess mutation profile of the subject).

In one embodiment, the quantified DNA methylation in a sample collected from a subject, quantified according to the methods described herein, can be incorporated into a clinical recommendation for the subject. A clinical recommendation can include a plan for further testing or treatment: For example, based on the quantified DNA methylation in the sample and the clinical implications of the DNA methylation, a plan for further testing or treatment can be developed. In some cases, this may involve additional testing to confirm the diagnosis, monitoring for disease progression or recurrence, or prescribing targeted therapies that are specific to the subject's DNA methylation profile.

### Quantifying DNA Methylation in Sample

This disclosure also features methods to quantify DNA methylation in a sample comprising DNA molecules.

In some embodiments, the method includes: treating the sample to encode the presence or absence of DNA methylation at loci of the DNA molecules. In other embodiments the sample has already been treated to encode the presence or absence of DNA methylation in the DNA molecules.

In some embodiments, the sample (e.g., the sample containing a mixture of methylated and unmethylated DNA sequences) may include at least ten target loci from the DNA sequences (e.g., loci that are amplified and assessed to quantify methylation). In some cases, each locus of the at least ten target loci is chosen due to an expected increase in DNA methylation at each loci in cancerous tissues compared to normal tissue. The sample can also include normalization loci (e.g., loci that are highly methylated in both cancerous tissues and normal tissue (e.g., non-cancerous tissue)). In some cases, the normalization loci are included in the target loci (e.g., the at least ten target loci). The method also includes adding to the sample a set of synthetic molecules that include target-associated regions having a nucleotide sequence that matches a target sequence region of an endogenous target molecule (wherein the endogenous target molecule comprises at least one of the target loci), having at least one of the target loci, and variation regions with sequence that does not match a sequence region of an endogenous target molecule. The method further includes generating a co-amplification mixture comprising an amplified set of synthetic molecules and an amplified set of at least ten target loci from the DNA sequences. In some embodiments, the co-amplification mixture comprises at least one normalization locus that is expected to have high methylation in cfDNA across both cancerous and non-cancerous tissues. The method includes sequencing the co-amplification mixture to generate sequence reads, and determining a number of the sequence reads that are methylated sequence reads. The method additionally includes quantifying (determining) a number (e.g., an absolute number) of methylated molecules in the sample (e.g., for each locus) based on the number of methylated reads from the sample (e.g., from each locus) and a number of reads from the set of synthetic molecules. In one embodiment, the method includes quantifying (determining) a number (e.g., an absolute number) of methylated molecules in the sample for each target loci based on the number of methylated reads from the sample for each target loci and a number of reads from the set of synthetic molecules. In some embodiments, the method further comprises aggregating the number of methylated molecules across at least two of the at least ten target loci to quantify the DNA methylation in the sample.

In another embodiment, this disclosure also features methods for quantifying DNA methylation in a sample containing cell free DNA (cfDNA) molecules. In some embodiments, the quantified DNA methylation is a tumor methylation score. The method includes treating the sample (e.g., wherein the sample is taken from a blood draw that includes plasma having cfDNA and buffy coat having genomic DNA (gDNA)) to encode the presence or absence of DNA methylation at loci of the DNA molecules (e.g., the cfDNA and/or the gDNA molecules). The sample (e.g., the sample containing a mixture of methylated and unmethylated DNA sequences) may include at least ten target loci from the DNA molecules (e.g., loci that are amplified and assessed for the presence of methylation). In some cases, each locus of the at least ten target loci is chosen due to an expected increase in DNA methylation at each loci in cancerous tissues compared to normal tissue (e.g., non-cancerous tissue). The sample can also include normalization loci (e.g., loci that are highly methylated in both cancerous tissues and normal (e.g., non-cancerous) tissue). A set of synthetic molecules is added to the sample, where the set of molecules include: target-associated regions having a nucleotide sequence that matches a target sequence region of an endogenous target, and variation regions with a nucleotide sequence that does not match a sequence region of an endogenous target molecule. The sample containing the synthetic molecules is amplified thereby generating a co-amplification mixture that includes an amplified set of synthetic molecules, and an amplified set of at least ten target loci from the DNA molecules, which optionally includes normalization loci. The method includes sequencing the co-amplification mixture at a read depth of at least one read sequence per molecule in the sample to generate sequence reads and determining a number of the sequence reads that are methylated sequence reads. The method also includes processing the methylated sequence reads using one or more of the following: filtering out selected hypermethylated target loci (e.g., filtering out target loci having a total number of methylated molecules in the buffy coat above a threshold); and/or subtracting background methylation (e.g., subtracting background methylation comprises subtracting the number of methylated molecules as measured in the buffy coat from the number of methylated molecules in the cfDNA (e.g., on a per-locus basis)). The method further includes quantifying (determining) a number (e.g., an absolute number) of methylated molecules in the sample based on the number of methylated reads from the sample and a number of sequence reads from the set of synthetic molecules. In one embodiment, the method includes quantifying (determining) a number (e.g., an absolute number) of methylated molecules in the sample for each target loci based on the number of methylated reads from the sample for each target loci and a number of reads from the set of synthetic molecules. In some embodiments, the method also includes aggregating the number of methylated molecules across at least two of the at least ten target loci to quantify DNA methylation in the DNA sample. In some embodiments, the sample is taken from a blood draw comprising plasma and buffy coat, where the plasma includes cfDNA molecules and the buffy coat comprises gDNA.

In one embodiment, the method includes determining (quantifying) the number of methylated molecules in cfDNA and/or gDNA using QCT molecular counting technology (e.g., as described in U.S. Pat. Pub. 2019/0211395A1, which is incorporated by reference) at the at least ten target loci, where each locus of the at least ten target loci is chosen due to an expected increase in DNA methylation at each loci in cancerous tissues compared to normal (e.g., non-cancerous) tissue. In such embodiments, the number of methylated molecules for cfDNA and the number of methylated molecules for gDNA are each quantified (e.g., in separate workflows) by treating the sample to encode the presence or absence of DNA methylation in loci of the DNA molecules, where the sample comprises at least ten target loci; adding to the sample a set of synthetic molecules, the set of molecules comprising: target-associated regions having a nucleotide sequence that matches a target sequence region of an endogenous target and variation regions with a nucleotide sequence that does not match a sequence region of an endogenous target molecule; generating a co-amplification mixture comprising an amplified set of synthetic molecules and an amplified set of at least ten target loci (or a subset thereof) from the DNA molecules; sequencing the co-amplified mixture (e.g., at a read depth of at least one read sequence per molecule in the sample) to generate sequence reads; determining a number of the sequence reads that are methylated sequence reads; and quantifying (determining) a number (e.g., an absolute number) of methylated molecules in the sample based on the number of methylated reads from the sample and a number of reads from the set of synthetic molecules.

In some embodiments, quantifying DNA methylation in a sample includes quantifying the number of methylated molecules for the cfDNA and the number of methylated molecules for gDNA, which can be quantified in the same workflow. In one embodiment, methylated molecules of cfDNA and methylated molecules of gDNA can be indexed (e.g., using defined index sequences) in order to be able to differentiate between the source of methylated molecules (e.g., cfDNA versus gDNA), thereby enabling the cfDNA and gDNA to be amplified and/or sequenced in the same reaction (e.g., multiplexing).

In one embodiment, quantifying DNA methylation in a sample containing gDNA molecules from buffy coat includes: treating a sample that includes methylated and unmethylated gDNA sequences from buffy coat to encode the presence or absence of DNA methylation in the gDNA sequences. In such cases, the sample includes at least ten target loci from the gDNA molecules, wherein each locus of the at least ten target loci is chosen due to an expected increase in DNA methylation at each loci in cancerous tissues compared to normal tissue and are the same target loci analyzed in the cfDNA extracted from plasma. The method includes adding to the sample a set of synthetic molecules, the set of molecules comprising: target-associated regions having a nucleotide sequence that matches a target sequence region of an endogenous target and variation regions with a nucleotide sequence that does not match a sequence region of an endogenous target molecule. The method also includes generating a co-amplification mixture comprising an amplified set of synthetic molecules and an amplified set of at least ten target loci from the DNA molecules and sequencing the co-amplification mixture to generate sequence reads. Further, the method includes determining a number of the sequence reads that are methylated sequence reads; and quantifying (determining) a number of methylated molecules in the sample based on the number of methylated reads from the sample and a number of reads from the set of synthetic molecules. Optionally, the method includes aggregating the number of methylated molecules across at least two of the at least ten target loci to quantify DNA methylation in the sample containing DNA molecules from the buffy coat.

In some embodiments, methods for quantifying DNA methylation in a sample include addition of a spike-in of known sequence and quantity to the sample prior to the treating step. The spike-in comprises a known sequence having unmethylated cytosines that are converted to uracils upon being subjected to the treating step. The initial number of spike-in molecules can be calculated as well as the percent of cytosine bases that were converted to thymine/uracil bases. This enables calculation of the bisulfite conversion yield and conversion efficiency, thereby establishing bisulfite conversion QC metrics, and therefore determining whether a sample failed the bisulfite conversion step (i.e., step of treating the sample containing DNA molecules to encode the presence or absence of DNA methylation at loci of the DNA molecules).

In some embodiments, the method for quantifying DNA methylation in a sample includes a step of processing the methylated sequence reads by one or more of the following: filtering out selected hypermethylated target loci (e.g., filtering out target loci having a total number of methylated molecules in the buffy coat above a threshold); or subtracting background methylation (e.g., subtracting the number of methylated molecules as measured in the buffy coat from the number of methylated molecules in the cfDNA, which can be done a per-locus basis). In some embodiments, processing is performed prior to the step of quantifying the number (e.g., the absolute number) of methylated molecules in the sample. In some embodiments, filtering hypermethylated target loci, subtracting background methylation, or both, are performed prior to quantifying the number of methylated molecules in the sample.

In some embodiments, methods for quantifying DNA methylation in a sample include a step of further comprising aggregating the number of methylated molecules across at least two of the at least ten target loci to quantify the DNA methylation in the sample. Aggregation can include aggregating target loci that contain lower than a threshold amount of methylated molecules in the buffy coat sample.

In some embodiments, methods for quantifying DNA methylation in a sample include aggregating the number of methylated molecules across the at least ten target loci (or a subset thereof, e.g., at least two target loci); and normalizing the aggregate number of methylated molecules from all or a subset of the at least 10 target loci by the methylated molecules for the at least one normalization locus. In some embodiments, this results in quantification of DNA methylation (e.g., a tumor methylation score) that represents the normalized sum of methylated molecules (e.g., at the at least ten target loci that are hypermethylated in the sample.

In some embodiments, aggregating the number of methylated molecules across the at least ten target loci (or a subset thereof, e.g., at least two target loci)) includes aggregating at least one target loci that demonstrates high methylation in the sample containing DNA molecules.

In one embodiment, the method further comprises determining cancer tissue of origin by quantifying methylation in a DNA sample, wherein determining the cancer tissue of origin comprises: determining the number of methylated molecules in the sample based on the number of methylated reads from the sample and the number of reads from the set of synthetic molecules at each locus; and determining the tissue of origin based on an abundance of methylated molecules across loci.

### Quantifying Tumor DNA in a Sample

This disclosure also features methods for quantifying the amount of tumor DNA in a sample containing DNA molecules. The method includes: adding to the sample (e.g., wherein the sample is taken from a blood draw that includes plasma having cfDNA and buffy coat having genomic DNA (gDNA)) a set of synthetic molecules, the set of molecules comprising: target-associated regions having a nucleotide sequence that matches a target sequence region of an endogenous target (e.g., comprising at least one target loci), and variation regions with a nucleotide sequence that does not match a sequence region of a endogenous target molecule. The method includes generating a co-amplification mixture comprising an amplified set of synthetic molecules, an amplified set of at least 10 target loci from the DNA molecules, and optionally an amplified one or more normalization locus; sequencing the co-amplification mixture at a read depth of at least one read sequence per molecule in the sample to generate sequence reads; quantifying (determining) a number of molecules containing a somatic mutation in the sample based on the number of reads from the sample containing a somatic mutation and the number of reads from the set of synthetic molecules.

The quantification of tumor DNA can be used to determine the tumor fraction in a sample. Tumor fraction measurements are useful indicators to monitor tumor growth and treatment effects.

### Determining a Methylation Profile in a Subject Over Time

This disclosure also features methods for determining a DNA methylation profile in a subject over time. Determining a methylation profile in a subject over time using serial quantification of DNA methylation enables early indication of the response or progression of the cancer to therapy (or reoccurrence). As provided described herein, the instant methods uniquely enable this type of serial quantification of DNA methylation.

In one embodiment, a method for determining a DNA methylation profile in a subject over time includes quantifying DNA methylation a first time point and a second time point; and determining whether the methylation (which is a proxy tumor measurement) has changed over time (e.g., increased or decreased compared to the measuring at a first time point). In some cases, the change in DNA methylation (i.e., Tumor Methylation Score) exceeds a significance threshold and is reported as an increase or a decrease. In some embodiments, the methods for determining a DNA methylation profile in a subject over time can distinguish a 0.2 percentage point change (or lower (e.g., 0.1, 0.05, 0.01, 0.05, 0.001 percent change) in DNA methylation (e.g., number of methylated molecules) with 3 standard deviations of separation.

In some embodiments, a method for determining a DNA methylation profile in a subject over time includes: i) treating a sample isolated from subject to encode the presence or absence of DNA methylation at target loci of the DNA molecules, wherein the sample comprises at least ten target loci from the DNA molecules, where each locus of the at least ten target loci is chosen based on an expected increase in DNA methylation at each loci in cancerous tissue compared to non-cancerous tissue and the sample optionally includes normalization loci (e.g., loci that are highly methylated in both cancerous tissues and normal tissue (e.g., non-cancerous) tissue); ii) adding to the sample a set of synthetic molecules (e.g., QCT molecules), the set of molecules comprising: target-associated regions having a nucleotide sequence that matches a target sequence region of an endogenous target molecule comprising at least one of the target loci, and variation regions with a nucleotide sequence that does not match a sequence region of an endogenous target molecule; iii) generating a co-amplification mixture comprising an amplified set of synthetic molecules and an amplified set of at least ten target loci from the DNA molecules; iv) sequencing the co-amplification mixture at a read depth of at least one read sequence per molecule in the sample to generate sequence reads; v) determining a number of the sequence reads that are methylated sequence reads; vi) quantifying (determining) a number of methylated molecules (e.g., for each target loci) in the sample based on the number of methylated sequence reads (e.g., from each target loci) in the sample and a number of reads from the set of synthetic molecules; repeating steps i) to vi) at a second time point; and determining the methylation pattern in the subject based on the number of methylated molecules (e.g., for each target loci) in the sample at the first time point and the number of methylated molecules (e.g., for each target loci) in the sample at the second time point. In some embodiments, the sample has already been treated to encode the presence or absence of DNA methylation at target loci in the DNA molecules, and therefore, step i) is not performed.

In some embodiments, determining the methylation profile in the subject at the first time point and the second time point identifies a change in the methylation profile. In one embodiment, methods for determining a DNA methylation profile in a subject over time include repeating steps i) to vi) at a third time point; and determining the methylation profile in the subject based on the number of methylated molecules (e.g., for each target loci) in the sample at the third time point.

In some embodiments, determining the methylation profile in the subject at the first time point, the second time point, and the third time point identifies a change in the methylation profile between the first and second time points, the second and third time points, the first and third time points, or a combination thereof.

In one embodiment, methods for determining a DNA methylation profile in a subject over time includes quantifying the number of methylated molecules in both cfDNA (plasma) and gDNA (buffy coat) using QCT molecular counting technology (e.g., as described in U.S. Pat. Pub. 2019/0211395A1, which is incorporated by reference) at the at least ten target loci, where each locus of the at least ten target loci is chosen due to an expected increase in DNA methylation at each loci in cancerous tissues compared to normal (e.g., non-cancerous) tissue. In such embodiments, the number of methylated molecules for cfDNA is quantified as described herein and the number of methylated molecules for gDNA are each quantified as described herein.

In some embodiments, methods for determining a DNA methylation profile in a subject overtime include addition of a spike-in of known sequence and quantity to the sample prior to the treating step at each time point. The spike-in comprises a known sequence having unmethylated cytosines that are converted to uracils upon being subjected to the treating step, which enables calculation of the bisulfite conversion yield and conversion efficiency, thereby establishing bisulfite conversion QC metrics that can be used to determine if a sample failed the bisulfite conversion step.

In some embodiments, methods for determining a DNA methylation profile in a subject over time include a step of processing the methylated sequence reads by one or more of the following: filtering out selected hypermethylated target loci (e.g., filtering out target loci having a total number of methylated molecules in the buffy coat above a threshold); or subtracting background methylation (e.g., subtracting the number of methylated molecules as measured in the buffy coat from the number of methylated molecules in the cfDNA, which can be done a per-locus basis). In some embodiments, processing is performed prior to the step of quantifying the number (e.g., the absolute number) of methylated molecules in the sample. In some embodiments, filtering hypermethylated target loci, subtracting background methylation, or both, are performed prior to quantifying the number of methylated molecules in the sample.

In some embodiments, methods for quantifying DNA methylation in a sample include a step of further comprising aggregating the number of methylated molecules across at least two of the at least ten target loci to quantify the DNA methylation in the sample. Aggregation can include aggregating target loci that contain lower than a threshold amount of methylated molecules in the buffy coat sample.

In some embodiments, methods for determining a DNA methylation profile in a subject over time include aggregating the number of methylated molecules across the at least ten target loci (or a subset thereof, e.g., at least two target loci); and normalizing the aggregate number of methylated molecules from all or a subset of the at least 10 target loci by the methylated molecules for the at least one normalization locus. In some embodiments, the aggregating and normalizing results in quantification of DNA methylation (e.g., a Tumor Methylation Score) that represents the normalized sum of methylated molecules (e.g., at the at least ten target loci that are hypermethylated in the sample at a given time point). Comparing across time points enables determination of a DNA methylation profile in a subject (over time). In some cases, the DNA methylation profile identifies a change in the methylation profile from the first time point to the second time points, second to the third time points, or the first to the third time points.

In some embodiments, a method for determining a DNA methylation profile in a subject overtime includes assigning a change in methylation profile a metric of: an increase, a decrease, or a no-change based on comparison to a significance threshold. The significance threshold can be predetermined or dynamically calculated. For example, without limitation, a method for determining a DNA methylation profile in a subject over time indicates a change where the change is an increase because the DNA methylation profile (e.g., the Tumor Methylation Score™) exceed a predetermined significance threshold of about 15% compared to a previous timepoint. For example, without limitation, a method for determining a DNA methylation profile in a subject over time indicates a change where the change is a decrease because the DNA methylation profile (e.g., the Tumor Methylation Score™) exceeds a predetermined significance threshold of about 15% compared to a previous timepoint. In some embodiments, the predetermined significance threshold to be exceeded for a change in a DNA methylation profile (e.g., the Tumor Methylation Score) to be considered an increase or a decrease is 20%, 19%, 18%, 17%, 16%, 15%, 14%, 13%, 12%, 11%, 10%, 9%, 8%, 7%, 6%, 5%, 4%, 3%, 2% or 1% compared to a previous timepoint.

In some embodiments, the change in methylation pattern indicates a change in a tumor in the subject. The change in the tumor comprises a change in the size of the tumor, a change in abundance of a somatic mutation associated with the tumor, a presence of a new somatic mutation associated with the tumor, a chromosomal abnormality associated with the tumor, or that the tumor is resistant to a therapy. In some embodiments, an increase in a DNA methylation profile (e.g., Tumor Methylation Score™) of at least 1.5-fold (e.g., 2-fold, 3-fold, 4-fold, or 5-fold) compared to a DNA methylation profile at a previous timepoint indicates a change in the tumor. In some embodiments, an increase in a DNA methylation profile (e.g., Tumor Methylation Score™) of at least 2-fold compared to a DNA methylation profile at a previous timepoint indicates a change in the tumor. In some embodiments, where DNA methylation in a sample includes at least 10% (e.g., at least 15%, at least 20%, at least 30%, at least 40%, at least 50%, at least 60%, at least 70%, at least 80%, or at least 90%) of the DNA methylation as originating from newly methylated target loci (e.g., a target loci not previously found to be methylated at a previous time point), the DNA methylation profile identifies a change in the tumor (e.g., the tumor associated with the sample). In some embodiments, where DNA methylation in a sample includes at least 40% of the DNA methylation in the sample (e.g., methylation at the target loci in cfDNA) of the DNA methylation as originating from newly methylated target loci (e.g., a target loci not previously found to be methylated at a previous time point), the DNA methylation profile identifies a change in the tumor (e.g., the tumor associated with the sample). In some embodiments, an increase in a DNA methylation profile (e.g., Tumor Methylation Score™) of at least 2-fold compared to a DNA methylation profile at a previous timepoint and where at least 40% of the DNA methylation in the sample (e.g., 40% of Tumor Methylation Score™) is from newly methylated target loci (e.g., a target loci not previously found to be methylated at a previous time point) indicate a change in the tumor (e.g., the tumor associated with the sample).

In one embodiment, a DNA methylation profile determined using the methods described herein can be incorporated into a clinical recommendation. A clinical recommendation can include a plan for further testing or treatment: For example, based on the quantified DNA methylation in the sample and the clinical implications of the DNA methylation, a plan for further testing or treatment can be developed. In some cases, this may involve additional testing to confirm the diagnosis, monitoring for disease progression or recurrence, or prescribing targeted therapies that are specific to the subject's DNA methylation profile.

This disclosure provides for methods of quantifying tumor methylation in an unbiased manner, i.e., without a priori information on the loci that need to be studied.

### Quantifying the Abundance of a Somatic Mutation in a Sample

This disclosure also features methods for determining an indication of confidence in the presence or absence of a somatic mutation in a sample where an indication of confidence in the presence or absence of a somatic mutation in the sample is assigned a “true call” or a “no-call” based, in part on, quantification of DNA methylation in the same sample from which the somatic mutation was determined to be present or absent. In one example, concordance between presence or absence of a somatic mutation and DNA methylation shows that methylation (i.e., methylation analyzed and identified according to the methods described herein) can be used to inform the confidence levels of whether a somatic mutation is present or absent, which in turn, can be used for a clinical recommendation for the subject.

In one embodiment, a method for determining an indication of confidence in the presence or absence of a somatic mutation in a sample containing DNA molecules, includes the following one or more steps. In some embodiments, the method includes a step of determining a presence or absence of a somatic mutation in the sample. In other embodiments, the method does not include a step of determining a presence or absence of a somatic mutation in the sample because the treating step had been done prior to use of the methods provided herein. In such cases, the method includes quantifying DNA methylation in the sample that was used to determine the presence or absence of the somatic mutation, but with the determination of the presence or absence of the somatic mutation performed prior to using the methods described herein.

In some embodiments, the method for determining an indication of confidence in the presence or absence of a somatic mutation in a sample containing DNA molecules includes: quantifying DNA methylation in the sample comprising the steps of as described herein. In some cases, the method includes optionally treating the sample to encode the presence or absence of DNA methylation at target loci of the DNA molecules, wherein the sample comprises at least ten target loci from the DNA molecules, where each locus of the at least ten target loci is chosen based on an expected increase in DNA methylation at each loci in cancerous tissue compared to non-cancerous tissue and the sample optionally includes normalization loci (e.g., loci that are highly methylated in both cancerous tissues and normal (e.g., non-cancerous) tissue). In some embodiments, the sample is treated to encode the presence or absence of DNA methylation in the DNA sequences prior to performing the methods described herein. In some embodiments, the method includes adding to the sample a set of synthetic molecules, the set of molecules comprising: target-associated regions having a nucleotide sequence that matches a corresponding nucleotide sequence to an endogenous target, and variation regions with a nucleotide sequence that does not match a sequence region of an endogenous target molecule. The method also includes generating a co-amplification mixture comprising an amplified set of synthetic molecules and an amplified set of at least ten target loci from the DNA molecules; and sequencing the co-amplified mixture (e.g., at a read depth of at least one read sequence per molecule in the sample to generate sequence reads). The method further includes determining a number of the sequence reads that are methylated sequence reads; quantifying a number of methylated molecules in the sample based on the number of methylated reads from the sample and a number of reads from the set of synthetic molecules.

Following determination of the presence or absence of the somatic mutation and the quantity of the DNA methylation in the sample, the method returns an indication of confidence (i.e., confidence in the determination of the presence or absence of the somatic mutation) as: a true call result for the presence or absence of the somatic mutation when the number of methylated molecules in the sample is above a predetermined or dynamically calculated threshold; or a no-call result for the presence or absence of the somatic mutation when the number of methylated molecules in the sample is at or below a predetermined or dynamically calculated threshold, whereby a true call identifies confidence in the determination of the presence or absence of the somatic mutation.

In one embodiment, methods for determining an indication of confidence in the presence or absence of a somatic mutation in a sample include quantifying the number of methylated molecules in both cfDNA (plasma) and gDNA (buffy coat) using QCT molecular counting technology (e.g., as described in U.S. Pat. Pub. 2019/0211395A1, which is incorporated by reference) at the at least ten target loci, where each locus of the at least ten target loci is chosen due to an expected increase in DNA methylation at each loci in cancerous tissues compared to normal (e.g., non-cancerous) tissue. In such embodiments, the number of methylated molecules for cfDNA is quantified as described herein (see Section 6.7) and the number of methylated molecules for gDNA are each quantified as described herein (see Section 6.7). The quantification of the number of methylated molecules provides a mechanism by which to assign the determination of the presence or absence of a somatic mutation a true call (e.g., a correct call) or a no-call (e.g., insufficient DNA to assign sufficient confidence to the abundance measurement). For example, quantifying DNA methylation in the same sample that was used to determine the presence or absence of the somatic mutations returns an indication of confidence for the somatic mutation determination. A true call result for the presence or absence of the somatic mutation is returned when the number of methylated molecules in the sample is above a predetermined or dynamically calculated threshold. A no-call result for the presence or absence of the somatic mutation is returned when the number of methylated molecules in the sample is at or below the predetermined or dynamically calculated threshold. A no call indicates that the determination of the presence or abundance of the somatic mutation cannot be assigned sufficient confidence to say the measurement is correct.

In some embodiments, methods for determining an indication of confidence in the presence or absence of a somatic mutation in a sample include addition of a spike-in of known sequence and quantity to the sample prior to the treating step. The spike-in comprises a known sequence having unmethylated cytosines that are converted to uracils upon being subjected to the treating step. As noted above, this enables calculation of the bisulfite conversion yield and conversion efficiency, thereby establishing bisulfite conversion QC metrics that can be used to determine if a sample failed the bisulfite conversion step.

In some embodiments, methods for determining an indication of confidence in the presence or absence of a somatic mutation in a sample include a step of processing the methylated sequence reads by one or more of the following: filtering out selected hypermethylated target loci (e.g., filtering out target loci having a total number of methylated molecules in the buffy coat above a threshold); or subtracting background methylation (e.g., subtracting the number of methylated molecules as measured in the buffy coat from the number of methylated molecules in the cfDNA, which can be done a per-locus basis). In some embodiments, processing is performed prior to the step of quantifying the number (e.g., the absolute number) of methylated molecules in the sample. In such cases, filtering hypermethylated loci, subtracting background methylation, or both, are performed prior to quantifying the absolute number of methylated molecules in the sample.

In some embodiments, upon returning a true call and where the presence of the somatic mutation indicates the presence of cancer, the method includes performing or repeating a treatment selection assay (e.g., treatment selection assay comprises genomic profiling to detect novel somatic mutations, the abundance of somatic mutations, or both) on the subject. In some embodiments, upon returning of a no-call, the method is repeated, at least in part, on a different sample taken from the same subject.

In some embodiments, the method for determining an indication of confidence in the presence or absence of a somatic mutation in a sample include a step of further comprising aggregating the number of methylated molecules across at least two of the at least ten target loci to quantify the DNA methylation in the sample. Aggregation can include aggregating target loci that contain lower than a threshold amount of methylated molecules in the buffy coat.

In some embodiments, the method for determining an indication of confidence in the presence or absence of a somatic mutation in a sample includes aggregating the number of methylated molecules across the at least ten target loci (or a subset thereof, e.g., at least two target loci); and normalizing the aggregate number of methylated molecules from all or a subset of the at least 10 target loci by the methylated molecules for the at least one normalization locus.

### Kit

In another aspect, this disclosure features a kit for quantifying DNA methylation in a sample using the methods described herein. In some embodiments, a kit for quantifying DNA methylation in a sample using the methods described herein. In some embodiments, a kit can include one or more of the following: conversion reagents required to encode the presence or absence of DNA methylation at target loci in DNA molecules, synthetic molecules (e.g., QCT molecules) to be added to the sample at various points in sample processing, reagents required for the amplification of the DNA sample, and reagents required for preparing a sequencing library such as those needed for indexing PCR.

This disclosure describes methods for measuring tumor fraction in cfDNA without requiring prior knowledge of tumor-specific alterations. This “tumor-naive” approach overcomes limitations of conventional methods that require tumor tissue analysis.

### Sample Collection and Processing

In one embodiment, the method begins with collection of a blood sample from a subject. The blood sample is separated into portions including a portion comprising plasma and a portion comprising buffy coat. DNA extraction is performed on each portion to obtain cfDNA from plasma and white blood cell (WBC) DNA from the buffy coat.

The cfDNA from the plasma and the WBC DNA from the buffy coat are subjected to bisulfite conversion, wherein CpG methylation sites remain unchanged while non-methylated cytosines are converted to uracils. This conversion allows for subsequent detection of methylation patterns by sequencing.

### Selective Enrichment and Absolute Quantification

Samples are selectively enriched at target loci, which may be CpG sites, DMR loci, or any other suitable target. In one embodiment, selective enrichment is performed using a multiplex PCR assay to specifically amplify the targer loci. Alternatively, selective enrichment may be performed using hybrid capture or any other suitable approach. In some embodiments, the assay targets more than 2,000 loci. The more than 2,000 loci may be differentially hypermethylated in cancer compared to normal cells.

In some embodiments, the assay incorporates Quantitative Counting Templates (QCT), which are synthetic DNA molecules with embedded molecular identifiers and PCR primer binding regions that co-amplify with the sample DNA. After PCR amplification, the sample is indexed and next-generation sequencing (NGS) is performed.

The QCT-derived sequencing reads are used to determine the number of sequencing reads per DNA molecule at each locus, which is then used for absolute quantification of sample-derived DNA. This method results in absolute quantification of methylated DNA at each of the multiple loci.

### Statistical Analysis for Tumor Fraction Determination

In a tissue-naive setting, it is not known a priori which of the loci are actually methylated in the tumor. Detecting a methylated DNA molecule at a locus provides evidence that the tumor is methylated at that locus. Yet, not detecting a methylated molecule could be either due to lack of methylation in that particular patient's tumor or sampling noise.

At high tumor burdens (e.g., >0.1% tumor fraction), the probability of obtaining 0 molecules in a locus is low, sampling noise is negligible, and the tumor fraction can be accurately obtained by averaging tumor fraction measurements across the detected loci. However, at low tumor burden, averaging across only detected loci would result in a severe over-estimate of TF. Alternatively, averaging across all loci (both detected and non-detected) would result in a severe under-estimate because many of the loci may not actually be methylated in the tumor.

To address this, in one embodiment, a zero-truncated Poisson (ZTP) statistical model is applied to the absolute counts of methylated DNA molecules detected across the loci. This approach does not require a priori knowledge of precisely which loci are methylated in the tumor. Rather, the proportion of loci at different non-zero levels (e.g., 1 molecule, 2 molecules, 3 molecules, etc.) is used to fit the average number of methylated DNA molecules across all loci.

The ZTP model enables accurate tumor fraction determination even when there is on average less than 1 molecule per locus, which is common in scenarios such as a typical blood sample containing only 1000 haploid genomic equivalents (GE) of cfDNA and a tumor fraction <0.1%.

### Improving Specificity by WBC Methylation Analysis

To improve specificity, some embodiments include an analysis of methylation patterns in the matched WBC DNA sample. Loci that exhibit methylation in the WBC sample are excluded from the analysis of the cfDNA sample.

Studies have shown that over 80% of the methylated cfDNA was found in loci that also had corresponding methylation in WBC. By analyzing the buffy coat and excluding the loci found to be methylated in WBC from the analysis of cfDNA, the specificity of the assay can be significantly improved.

### Background Subtraction for Improved Accuracy

Further improvements in accuracy are achieved through background subtraction. In one embodiment, a ZTP fit is applied to WBC measurements to estimate the true rate of WBC methylation, even among loci that did not detect any methylated DNA molecules due to sampling effects.

To obtain a “background subtracted” TF measurement, the ZTP TF measurement is scaled in proportion to the expected amount of methylation from WBC. This correction is applied using the formula: (methyl_cfdna−methyl_bg)/methyl_cfdna, where methyl_cfdna is the sum of methylated cfDNA molecules per input GE, and methyl_bg is the expected sum of non-tumor methylated molecules per input GE.

### Haplotype Block Analysis

In some embodiments, the method further includes grouping multiple loci into “haplotype blocks” to further improve tumor fraction accuracy. Loci blocks (e.g., 4 loci each) are assigned to represent a haplotype block, based on the principle that tumor-specific DNA methylation should co-occur across all loci within each haplotype block.

Within each block, the absolute counts of methylated DNA from the multiple loci are summed to obtain a count of methylated DNA molecules for that block. The ZTP method is then performed on loci blocks rather than individual loci. Grouping loci into blocks significantly improves the tumor fraction accuracy, especially at low tumor fractions.

### Applications

The methods described above have numerous applications in cancer management:

The variant allele fraction of somatic alterations detected in ctDNA can be compared with the overall ctDNA fraction to assess whether the somatic variant is consistently present in all tumor cells or if the somatic variant is only present in a subclonal population.

This information can be valuable for detecting the emergence of resistant subpopulations indicated by a resistance mutation (e.g., EGFR T790M is resistant to EGFR tyrosine kinase inhibitor), or in treatment decisions such as applying a therapy targeted against particular alterations.

The ctDNA tumor fraction can also be used to assess absence of a mutation, which is useful in certain clinical contexts. For example, KRAS wild-type colorectal cancer is treated differently than KRAS mutated cancer. An accurate determination of KRAS wild-type status is only possible when the ctDNA methylation fraction exceeds the limit of detection threshold for KRAS mutations.

### EXAMPLES

### Materials and Methods for Examples 1-10

### Tumor Hypermethylation Target Selection

The Cancer Genome Atlas's (TCGA) was queried for subjects with human methylation data for both tumor and normal tissue of the same tissue type. Data from TCGA was collected using the Infinium HumanMethylation450 BeadChip (Illumina, San Diego, CA) and provided beta values representing the methylation fraction at specific CpG locations in the genome. In addition, methylation data for white blood cells collected from patients of age similar to cancer patients (mean=63.9 years, sd=13.3 years) was obtained from GEO accession GSE40279 (see Hannum, G. et al., (2013). Molecular Cell, 49(2), 359-367. doi.org/10.1016/j.molcel.2012.10.016).

Tumor hypermethylation was calculated at each CpG site by subtracting normal tissue beta from tumor beta. To avoid choosing CpG sites with spurious hypermethylation, an average hypermethylation was calculated for each CpG island, and CpG islands were ranked in order of highest hypermethylation. Additionally, CpG islands were filtered for average white blood cell beta <0.2 to minimize background signal from buffy coat contributions to the cfDNA. From each selected CpG island, several CpG locations were chosen for primer design.

A lung cancer specific assay was designed using TCGA data from subjects with lung adenocarcinoma and lung squamous cell carcinoma. A pan-cancer assay was also designed using TCGA data from many cancer types (e.g., lung adenocarcinoma, lung squamous cell carcinoma, colon adenocarcinoma, breast invasive carcinoma, pancreatic adenocarcinoma, liver hepatocellular carcinoma, bladder urothelial carcinoma, esophageal carcinoma, kidney renal cell carcinoma, kidney renal papillary cell carcinoma, prostate adenocarcinoma, thyroid carcinoma, and uterine corpus endometrial carcinoma).

### Highly Methylated Target Selection

In addition to selecting targets with hypermethylation in tumors compared to normal tissue, control loci that are highly methylated in both buffy coat, tumor, and normal tissue were selected as well. Methylation data was obtained and analyzed similarly as for the hypermethylated targets, but ranked in order of highest white blood cell methylation signal and filtered for beta >0.9 for white blood cell, tumor tissue, and normal tissue for six cancer types (lung adenocarcinoma, lung squamous cell carcinoma, colon adenocarcinoma, breast invasive carcinoma, pancreatic adenocarcinoma, liver hepatocellular carcinoma).

### Primer and QCT Design

Primer3 was used to design primer pairs targeting the genomic locations of chosen CpG locations and using a reference genome that was bisulfite converted in silico. This process was performed assuming full methylation of the reference hg19 human genome, converting all Cs to Ts except for CpGs. The ideal annealing temperature was set to 60 °C

Single stranded QCTs were designed for each amplicon. The predicted amplicon sequence for each primer pair was determined using Bowtie and the converted human genome, and 17 bases of flanking genomic sequence were added to both 5′ and 3′ ends. Eleven bases in the insert region of the QCT were replaced with Ns, allowing for a small number of unique QCTs to be added to each PCR reaction.

### Additional Amplicons

In addition to tumor hypermethylated and highly methylated genomic targets, additional amplicons were included in the assay. Fifteen genotyping amplicons were designed targeting A>G or G>A SNPs that are commonly found according to NCBI's Single Nucleotide Polymorphism Database (dbSNP). The genotype at these locations can be used to check for sample swaps that may occur during lab processing or at the clinic. Three amplicons targeting genomic locations on chromosome Y were included to determine the sex of the patient.

Two artificial amplicons were included to check for bisulfite conversion yield and conversion efficiency. These synthetic oligos are spiked-in to the cfDNA and gDNA sample just prior to bisulfite conversion at a fixed quantity. These oligos are then amplified in the multiplex reaction along with all the other amplicons. During analysis, the initial number of spike-in molecules can be calculated as well as the percent of cytosine bases that were converted to thymine bases (the oligos were synthesized without any methylation). For the multiplex PCR no-template control, the same amount of bisulfite conversion spike-ins are added after bisulfite conversion, allowing for an estimate of the overall bisulfite conversion yield. Calculating the bisulfite conversion yield and conversion efficiency allows for the establishment of bisulfite conversion QC metrics and therefore determination of whether a sample failed the bisulfite conversion step of the process.

### Specimen Sourcing

To obtain a number of tumor specimens spanning a variety of cancer types, banked flash frozen tumors and buffy coats from the same subjects were obtained from Spectrum Health (Grand Rapids, MI).

To test whether the assay could detect changes in methylation that were concordant with clinical outcomes, samples were collected from cancer patients both retrospectively and prospectively. Retrospective samples consisting of banked plasma and buffy coat samples were obtained through collaborations with the University of California at San Diego and the University of Florida. Prospective sample collection was performed through contract research organizations and their partner clinics; patients that were diagnosed with cancer and had not started treatment were enrolled. Blood was collected in Streck tubes pre-treatment and at subsequent time points post-treatment. Clinical outcomes were provided when available. To test how the assay performs on healthy individuals, blood was collected from healthy volunteers at various time points.

### Specimen Preparation

Blood collected in EDTA tubes were spun down within one hour of collection and plasma and buffy coat were isolated. Blood collected in Streck tubes were allowed to sit overnight before spinning down and isolating plasma and buffy coat. Plasma volume was recorded for normalization in analysis.

cfDNA was extracted using the QIAamp Circulating Nucleic Acid Kit (Qiagen), and gDNA was extracted from tumor samples and buffy coat samples using the DNeasy Blood & Tissue Kit (Qiagen).

Contrived samples mimicking cell-free DNA (cfDNA) samples from cancer patients were created by mixing tumor genomic DNA (gDNA) into buffy coat gDNA at various tumor fractions.

### Bisulfite Conversion and Library Prep for Next Generation Sequencing

Samples were either bisulfite converted using the Diagenode Premium Bisulfite kit (Cat No. C02030030) or the Zymo EZ-96 DNA Methylation-Lightning MagPrep kit (Cat No. D5046). When either cfDNA or gDNA sample volumes were larger than the recommended input volume, samples were split in half, converted separately, and then re-combined. Enzymatic conversion was also tested. As a positive control for detecting methylation, universally methylated genomic DNA (Cat. No.S7821, Sigma-Aldrich) was diluted in buffy coat at various tumor fractions.

Primer mixes were created by pooling all primer pairs and iteratively removing and/or rebalancing the concentrations of each primer pair to optimize for balanced coverage across target amplicons. QCTs were diluted to 200 molecules per PCR reaction at each amplicon. A total of 113 target amplicons were included for the lung cancer assay, and 679 target amplicons were included for the pan-cancer assay.

Multiplex PCR was performed on bisulfite converted specimens using Q5U polymerase (NEB, Ipswich, MA). Subsequently, indexing PCR was performed using Q5 polymerase (NEB, Ipswich, MA) in order to sequence multiple samples on the same sequencing run with dual indexes. Pooled libraries were bead cleaned and loaded on NextSeq 2000 (Illumina, San Diego, CA) sequencing instruments using P3 100 cycle reagents for single-directional sequencing and 5% PhiX.

### Calculating the Number of Methylated Molecules

Fastq files were adapter trimmed on the 3′ end using BBDuk and then mapped using BWA-MEM to a custom genome composed of the target hypermethylated and highly methylated amplicon, and QCT molecules.

For each amplicon, reads that mapped to the target amplicon (e.g., the target hypermethylated amplicon or the highly methylated amplicon) were binned based on sequence. The number of CpGs contained in each sequence was calculated, and each sequence was classified as belonging to a methylated read if the number of CpGs was greater than or equal to the maximum number of possible CpGs for that amplicon minus one. Reads mapping to the corresponding QCT sequence were separately processed and binned based on the random N sequence of the QCT. Assuming each QCT molecule's sequence is unique in each reaction, an average number of reads per QCT molecule was calculated. The total number of methylated molecules for that amplicon can then be calculated by dividing the total number of methylated reads by the average reads per QCT molecule.

When measurements were obtained for paired cfDNA and buffy coat samples, background levels of methylation were reduced by subtracting the buffy coat methylation signal from the cfDNA methylation signal on a per-locus basis. It is estimated that about 55% of the cfDNA is of white blood cell origin based on methylation patterns (see Moss, J. et al., (2018), Nature Communications, 9(1), doi.org/10.1038/s41467-018-07466-6), suggesting that subtracting the entirety of the buffy coat methylation is a conservative approach to remove background methylation signal. Any calculated negative numbers of molecules after background subtraction were capped at zero.

In order to perform a comparable subtraction between buffy coat and cfDNA samples, the samples must first be normalized to the input amount of genomic equivalents. The average of the highly methylated loci methylated molecules is used to estimate the total number of genomic equivalents in the sample overall. The number of methylated molecules at each locus is normalized by the estimated genomic equivalents of that sample. The number of normalized methylated molecules is then used for background subtraction.

### Hypermethylation Locus Filtering

Based on testing healthy subjects, certain hypermethylation loci were found to have significant methylation signal even after buffy coat subtraction. To minimize the amount of false cancer signal, certain loci were filtered out from analysis. In some cases, this is referred to herein as “blacklisting.” Any loci found to either consistently contribute a moderate amount of methylation or occasionally contribute a high amount of methylation in healthy subjects were added to a list of loci to ignore. These loci often contain high amounts of methylation in buffy coat.

After filtering hypermethylation loci, the results from healthy subjects was used to establish a noise floor, below which the methylation signal is not interpretable because it is comparable to the amount seen in healthy subjects.

### Calling Changes in Methylation

When two time points of data were available, a call was made as to whether there has been an increase, decrease, or no change in the amount of methylated molecules. The call was made by modeling each time point's measurement as a normal distribution with a mean of the number of measured methylated molecules (normalized to the total number of input molecules). A standard deviation is assigned to the normal distribution based on the total number of methylated molecules; this can be done by interpolating based on previous studies where the coefficient of variation was measured from contrived samples (see, e.g., Example 7, Arm 3). The normal distribution from the first time point is subtracted from the second time point and normalized to the mean of the first time point's normal distribution, creating a normalized difference normal distribution. An “Increase” call was made if the mean of this distribution is ≥15% and the log 2 likelihood ratio that the difference was ≥15% compared to <15% was greater than 3. Similarly, a “Decrease” call was made if the mean of this distribution is ≤−15% and the log 2 likelihood ratio that the difference was ≤−15% compared to >−15% is greater than 3. If the relative difference was not of sufficiently large magnitude or the statistical likelihood was not sufficiently strong, a “No Change” call was made. If either time point was below the noise floor, the mean for that time point was set to the noise floor, the standard deviation was still calculated based on the total number of methylated molecules, and a call was still made. If the methylation signal from both time points was below the noise floor, an “Indeterminate” call was made. Any other suitable statistical methods, including but not limited to mean, median, standard deviation, likelihood ratio, expectation maximization, statistical significance tests can be used to make the calls.

### Methylation Profile

To capitalize on the fact that the assay measures methylation at hundreds of hypermethylation locations, a methylation profile was determined at each time point. One way to build the methylation profile was to categorize loci based on which time point a locus was first found to have informative methylation signal. This was defined as greater than 2 molecules after background subtraction. Categorizing the loci by first informative time point revealed when and how much the methylation profile changed.

### Example 1. Lung Cancer Assay can Detect Significant Methylation in 0.5% Tumor Fraction Contrived Samples

The lung cancer assay was assessed for its limit of detection using contrived samples. Without wishing to be bound by theory, contrived specimens are prepared to mimic clinical specimens as closely as possible. For these experiments, the contrived samples included sheared tumor gDNA, which were sheared using a sonicator to an average fragment length of ~170 bp to mimic the size distribution of cfDNA. Contrived samples made with universal methylation were used as a positive control (e.g., in place of tumor DNA as a positive control). Positive controls were sheared in the same manner as the contrived lung cancer samples. A total of 1.35E9 sequencing reads were obtained, resulting in an average of 11E6 average reads per sample. There were an average of 62 reads per QCT across all samples and amplicons. Each contrived sample was created with 5000 genomic equivalents (g.e.).

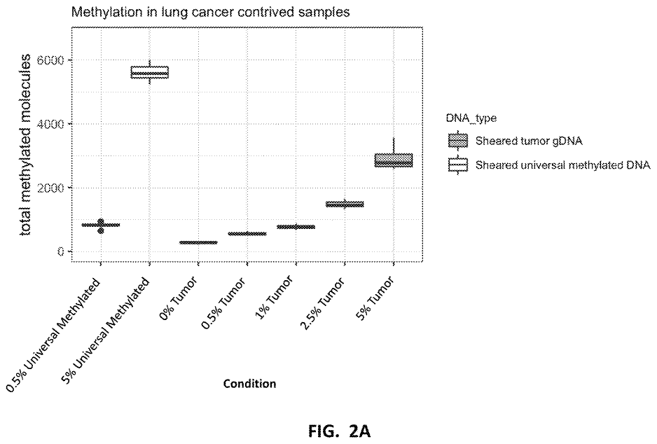

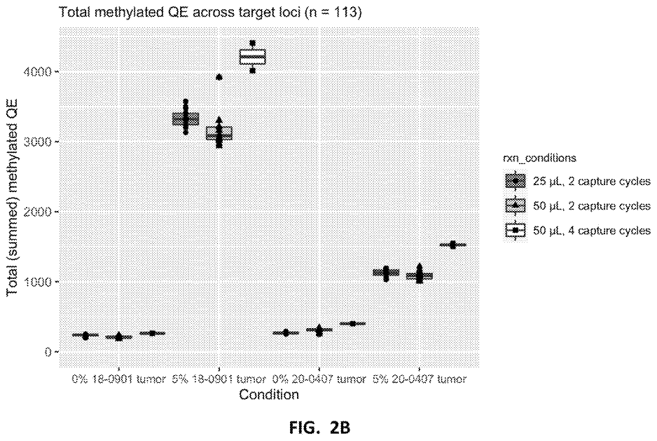

Technical replicates of lung cancer contrived samples were tested at 0%, 0.5%, 1%, 2.5%, and 5% tumor fractions (See FIG. 2A). The assay was able to distinguish 0.5% tumor fraction samples from 0% tumor fractions, suggesting that the limit of detection was at least 0.5%.

The lung cancer assay was also used to measure methylation in additional lung tumor specimens (see FIG. 2B). FIG. 2B shows the lung cancer assay was used to detect methylation in contrived samples from two additional lung cancer subjects. For these experiments different PCR volumes and different thermal cycling protocols were used (see FIG. 2B).

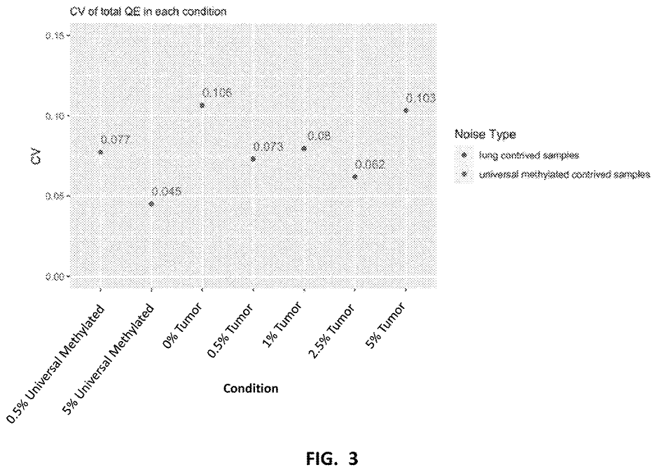

Additional analysis using the lung cancer assay included measuring concordance values (CV) (FIG. 3) of the samples analyzed in FIG. 1A. CVs were used to determine whether ctDNA, and correspondingly the amount of tumor, truly increased or decreased or whether the increase or decrease was false positive or a false negatives. Without any additional post-processing, the CV of the methylation measurements was around 10% or less. This suggested that the methylation measurements generated using the methods described herein confidently identified a 30% change in signal within 3 standard deviations. According to RECIST guidelines, a partial response was defined by at least a 30% decrease in the longest diameter of the target tumor lesion, and progressive disease was defined by at least a 20% increase in the longest diameter of the target tumor lesion. In view of these RECIST guidelines, the assays described herein enable assessment of tumor response to therapy in the context of current standard of care. In fact, quantification of treatment response following RECIST guidelines is limited by the selection of a limited number of identifiable tumors on imaging, whereas the methylation assay provides a measurement of all tumors present in the body that shed DNA into the bloodstream.

### Example 2. Background Subtraction Improves Signal-to-Background Ratio

Optimizations were performed to improve the signal-to-background ratio. Without wishing to be bound by theory, improving the signal-to-background ratio is particularly helpful for detecting smaller tumor signals. For example, one medical application is for minimal residual disease (MRD) detection, where the difference between having any tumor signal and zero signal could be the difference between recurrence and remission.

Despite filtering for targets with low methylation in buffy coat and normal tissue in the target selection process, some targets may still have significant amounts of background methylation. One approach to reduce the impact of background methylation was to mask the signal from target loci with buffy coat methylation above a certain threshold. Another parallel approach was to subtract the methylation signal as measured in the buffy coat from the methylation signal measured in the cfDNA (in the plasma) on a per-locus basis.

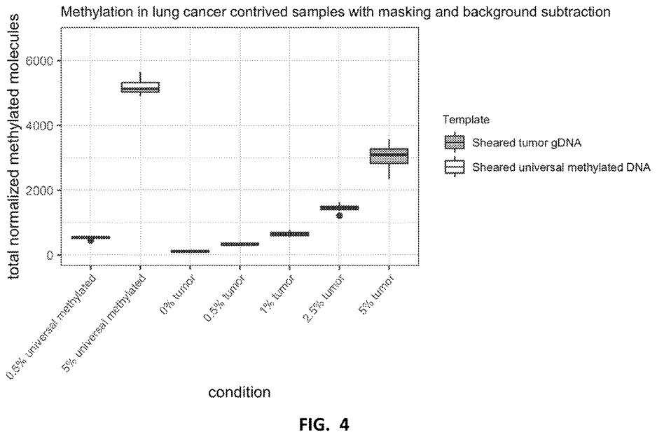

FIG. 4 shows total normalized methylated molecules for technical replicates of lung cancer contrived samples at various tumor fractions. In particular, FIG. 4 shows data from a lung cancer assay that included background subtraction (i.e., subtracting the methylation signal as measured in the buffy coat from the methylation signal measured in the cfDNA) and masking of target loci with high background methylation (i.e., mask the signal from target loci with buffy coat methylation above a certain threshold). For example, the background methylation signal was calculated by using the buffy coat methylation signal per locus, normalized to the average highly methylated loci methylated molecules (e.g., average of normalization loci) in each buffy coat sample, and averaging across all buffy coat samples. 55% of the background signal was subtracted from each cfDNA contrived sample's methylation signal, normalized to the average highly methylated loci methylated molecules in each contrived sample. Hypermethylation target loci with greater than 10 methylated molecules per 1000 highly methylated loci methylated molecules were masked. Any other suitable threshold can be determined.

By masking loci and subtracting the buffy coat methylation signal from the contrived cfDNA signal, background signal was reduced in the 0% tumor fraction contrived samples from an average of 277 methylated molecules to 111 methylated molecules. More stringent masking of hypermethylation target loci was expected to further reduce the background signal.

Overall, this data showed signal-to-background ratio could be improved using background subtraction (i.e., subtract the methylation signal as measured in the buffy coat from the methylation signal measured in the cfDNA) in parallel with masking of target loci with high background methylation (i.e., mask the signal from target loci with buffy coat methylation above a certain threshold).

### Example 3. Hypermethylation-Based Measurements are Robust to gDNA Contamination, Unlike Somatic Mutation-Based Approaches

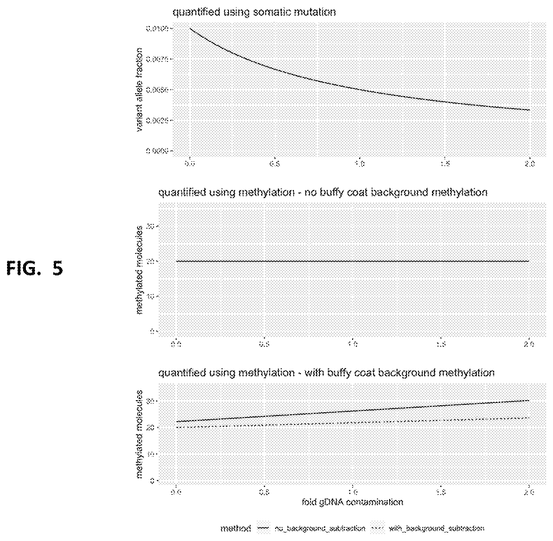

A common problem with cfDNA assays is gDNA contamination. This can occur during the plasma isolation of centrifuged whole blood when buffy coat is accidentally isolated along with the plasma fraction. gDNA contamination can have a detrimental effect on the accuracy of cfDNA tumor quantification especially when using somatic mutation variant allele fractions for cfDNA tumor quantification. This is because the additional buffy coat contributes to the denominator. As shown in FIG. 5 (top panel), 1×gDNA contamination (e.g., contamination that amounts to a doubling of the total number of molecules) reduced the variant allele fraction by half. This could result in a false positive signal. When a positive signal correlates with treatment efficacy, such a result has the potential to alter medical decision making.

This experiment showed that detection of tumor hypermethylation was less vulnerable to gDNA contamination than a somatic mutation ctDNA assay. This is at least because targets were selected for low buffy coat methylation and background subtraction methods were used. If the background methylation signal was zero in the buffy coat, the absolute number of methylated molecules was not affected at all by gDNA contamination (FIG. 5; middle panel). Even if buffy coat did contribute a small amount of methylation background signal (FIG. 5; bottom panel), measuring the buffy coat methylation and subtracting that signal from the cfDNA signal kept the total methylation signal within an increase of 20% for a 1× amount of gDNA contamination. This minimized the effect of gDNA contamination on the tumor methylation measurement and any potential downstream medical decisions to be made based on the tumor methylation measurement.

In addition to target selection and background subtraction, additional biochemical methods to minimize gDNA contamination existed, including bead purification to size select the DNA fragments in a cfDNA sample. However, as these methods can also fail, it is helpful to have orthogonal approaches for robustness against gDNA contamination.

### Example 4. Day-to-Day Variability in Buffy Coat Methylation in Healthy Subjects was Comparable to Subject-to-Subject Variability

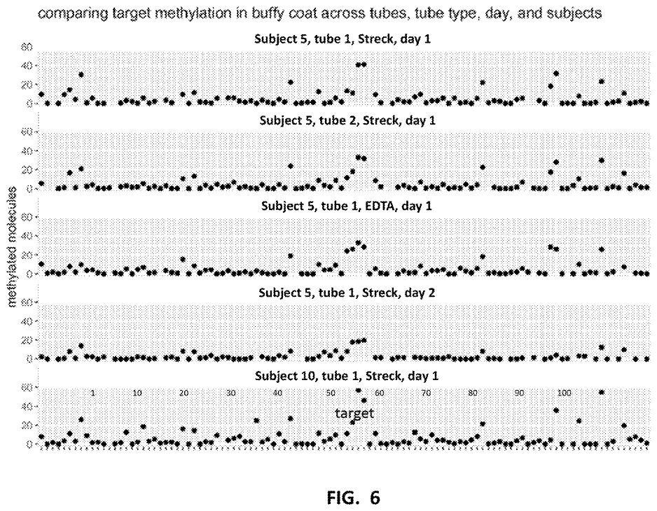

The degree of day-to-day variability of methylation in healthy subjects was assessed as part of the assay's ability to make accurate, time serial measurements. Because a significant portion of the cfDNA signal is of buffy coat origin, the methylation profile of buffy coats was measured from healthy subjects across different tubes of the same blood draw, different tube types, different days, and different subjects. The data in FIG. 6 shows methylation profiles for buffy coats (i.e., 5000 g.e. of buffy coat) isolated from different tubes of the same blood draw, different tube types, different days, and different subjects (see Table 1 for sample identifiers in FIG. 6).

TABLE 1

| | Column in FIG. 6 |
|---|---|
| Sample identifiers | (left to right) |
| BARHL2_cg11823511_3 | 1 |
| BNC1_MSP_W_0 | 2 |
| C2orf40_cg17161250_0 | 3 |
| C3orf72_cg184′4754_96 | 4 |
| CCDC140_cg12973118_0 | 5 |
| CNPY1_cg01198033_73 | 6 |
| CNPY1_cg07416383_4 | 7 |
| CTNNA2_cg20072442_66 | 8 |
| CTNNA2_cg24632241_54 | 9 |
| CYP11A1_cg16332610_29 | 10 |
| DIDO1_cg15680020_66 | 11 |
| DNM3_cg02011074_79 | 12 |
| DNM3_cg23391785_0 | 13 |
| FAIM2_cg18486102_2 | 14 |
| FAM59B_cg00399175_2 | 15 |
| FLJ32063_cg00768993_63 | 16 |
| FLJ32063_cg08690859_17 | 17 |
| FLJ32063_cg19376851_3 | 18 |
| GABRA4_cg24154839_2 | 19 |
| GRIA4_cg04747226_MSP_W_8 | 20 |
| HOXA9_cg15506609_0 | 21 |
| HOXB4_cg07438617_0 | 22 |
| HOXD8_cg14473102_31 | 23 |
| HOXD8_cg24416513_0 | 24 |
| INA_cg18932798_0 | 25 |
| intron_cg00158528_24 | 26 |
| intron_cg01447112_0 | 27 |
| intron_cg02340083_36 | 28 |
| intron_cg03075534_3 | 29 |
| intron_cg03204678_4 | 30 |
| intron_cg03257575_2 | 31 |
| intron_cg04067139_29 | 32 |
| intron_cg04502985_0 | 33 |
| intron_cg051838931_52 | 34 |
| intron_cg06643013_0 | 35 |
| intron_cg07149609_21 | 36 |
| intron_cg07891531_0 | 37 |
| intron_cg08189989_0 | 38 |
| intron_cg08235161_10 | 39 |
| intron_cg08368617_77 | 40 |
| intron_cg091929100_3 | 41 |
| intron_cg10034364_0 | 42 |
| intron_cg10422777_0 | 43 |
| intron_cg11071231_35 | 44 |
| intron_cg11665991_32 | 45 |
| intron_cg12505170_12 | 46 |
| intron_cg12853633_0 | 47 |
| intron_cg13239420_0 | 48 |
| intron_cg13368519_50 | 49 |
| intron_cg13713830_0 | 50 |
| intron_cg14189141_0 | 51 |
| intron_cg15415452_29 | 52 |
| intron_cg17869514_5 | 53 |
| intron_cg18144593_0 | 54 |
| intron_cg18969232_75 | 55 |
| intron_cg19025113_83 | 56 |
| intron_cg19375537_16 | 57 |
| intron_cg19516105_0 | 58 |
| intron_cg19737787_0 | 59 |
| intron_cg20785796_0 | 60 |
| intron_cg21235151_0 | 61 |
| intron_cg21821214_0 | 62 |
| intron_cg22524657_0 | 63 |
| intron_cg22876812_0 | 64 |
| intron_cg23089825_1 | 65 |
| intron_cg23348270_4 | 66 |
| intron_cg23713079_16 | 67 |
| intron_cg25168494_20 | 68 |
| intron_cg25570913_MSP_W_0 | 69 |
| intron_cg25738714_43 | 70 |
| intron_cg25739043_10 | 71 |
| intron_cg25950325_84 | 72 |
| intron_cg26379859_2 | 73 |
| intron_cg27315333_0 | 74 |
| intron_cg27529871_3 | 75 |
| intron_cg27555582_0 | 76 |
| ITGA8_cg16422098_99 | 77 |
| ITGA8_cg26104297_1 | 78 |
| L1TD1_cg03731268_79 | 79 |
| LHX1_cg14754787_18 | 80 |
| NPTX2_MSP_W_9 | 81 |
| PAX3_cg09424526_0 | 82 |
| PAX3_cg14265823_29 | 83 |
| PAX9_cg01672943_0 | 84 |
| PAX9_cg01972418_8 | 85 |
| PITX2_MSP_W_37 | 86 |
| PPFIA3_cg13484549_6 | 87 |
| PRDM14_cg11229513_5 | 88 |
| PROM1_cg12839172_14 | 89 |
| PTGDR_cg18693395_0 | 90 |
| PTGDR_cg24989962_0 | 91 |
| RALYL_cg25757598_0 | 92 |
| RNF220_cg18180569_7 | 93 |
| RYR2_cg07790615_4 | 94 |
| SFRP1_MSP_W_93 | 95 |
| SOX2OT_cg14317285_0 | 96 |
| SOX2OT_cg17202313_55 | 97 |
| TAC1_MSP_W_39 | 98 |
| TFPI2_MSP_W_92 | 99 |
| TMEM132C_cg00579520_16 | 100 |
| TMEM132C_cg03530754_97 | 101 |
| USP44_cg13879483_MSP_W_3 | 102 |
| USP44_cg22538054_MSP_W_3 | 103 |
| VAX1_cg17138769_45 | 104 |
| VWC2_cg01893212_MSP_W_29 | 105 |
| WT1_cg19126300_30 | 106 |
| ZNF781_cg25324105_0 | 107 |
| ZNRF3_cg13298692_18 | 108 |

For example, the two tubes from Subject 5 from the same day with the same tube type were very similar, which was expected (see FIG. 6). Tube type did not cause significant differences as the EDTA tube was very similar to both Streck tubes. The data from the Streck tube collected on Day 2 was similar to data from the Streck tube collected on Day 1, but with a few loci with notable differences. There were noticeable subject-to-subject differences, as Subject 10 had several methylated loci that were not methylated in any of the tubes from Subject 5.

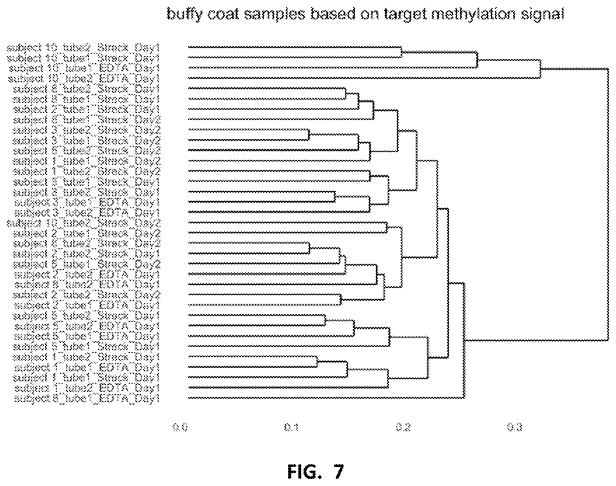

FIG. 7 shows hierarchical clustering of methylation profiles from buffy coats isolated from different tubes of the same blood draw, different tube types, different days, and different subjects. Clustering was performed based on methylated molecules at each target hypermethylation locus, normalized to the amount of methylated molecules measured at highly methylated loci. Cluster distance was calculated using the L1 norm.

Hierarchical clustering revealed that the tubes collected on the same day, no matter the tube type, clustered together the closest (see FIG. 7). Examples of tubes on the same day clustering together included Subject 1 Day 1, Subject 3 Day 1, Subject 3 Day 2, and Subject 10 Day 1. However, tubes collected from the same subject but on different days cluster as far apart as tubes collected from different subjects. This suggested that background subtraction was more optimal if performed using methylation profiles from buffy coat collected at each unique time point.

### Example 5. Pan-Cancer Assay Detects Methylation Signal in Multiple Cancer Types

A pan-cancer assay was designed based on the methods described herein.

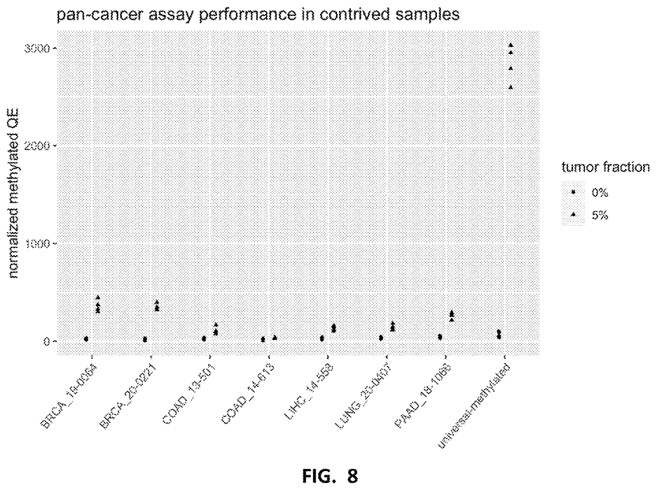

FIG. 8 shows data from the methylation signal in the pan-cancer assay in contrived samples of several different cancer types. For these experiments, 5% tumor fraction contrived samples were made by mixing 5% of tumor gDNA with 95% of buffy coat from the same subject by mass. 0% tumor fraction contrived samples were pure buffy coat. Specimens were labelled with their cancer type (BRCA: breast invasive carcinoma, COAD: colon adenocarcinoma, LIHC: liver hepatocellular carcinoma, LUNG: lung cancer, PAAD: pancreatic adenocarcinoma). Universal methylated samples were included as positive control for the methylation assay.

As shown in FIG. 8, the pan-cancer assay was able to detect methylation signals in 5% tumor fraction contrived samples of several cancer types using the same chemistry for all samples. There was noticeable variability in methylation signal across specimens, which could have been due to a couple causes. First, the methylation profile of the tumor likely differed from tumor to tumor. This profile may or may not have coincided with the target locations included in the assay. In the assays described herein, increasing the number of target locations can help minimize the effect of biological variability in methylation across tumors. The variability could have also been due to a technical limitation with contrived samples made with tumor specimens, as the tumors themselves contained an unknown proportion of normal tissue. Therefore, comparing measurements made on contrived samples of the same tumor fraction across specimens should be done cautiously. Despite the exact tumor quality being unknown, the assay was still able to differentiate the 5% tumor fraction contrived tumor samples from a pure buffy coat sample from the same subject.

Overall, this data established that the methods described herein include the ability for pan-cancer detection.

### Conclusion from Examples 1-5

This data demonstrated an innovative approach to cancer treatment monitoring by quantifying the absolute number of methylated molecules. Existing ctDNA assays have poor precision for treatment monitoring due to a very limited number of detectable variants and therefore a very small total number of variant molecules. Whole exome sequencing of a tumor biopsy could be performed to identify additional variants and overcome molecule sampling limitations, but that requires a tumor biopsy which may be difficult to obtain at all from the patient or may not have sufficient material remaining from the clinic. By resolving several technical shortcomings of existing treatment monitoring approaches, we believe that this methylation-based, tumor-naive, multiplexed, quantitative assay will be of great use to oncologists and their patients for guiding treatment decision making.

### Example 6. Filtering Hypermethylated Loci

The lung-cancer assay described in Example 2 improved the signal-to-background ratio using background subtraction, which included subtracting the methylation signal as measured in the buffy coat from the methylation signal measured in the cfDNA, in parallel with masking of target loci with high background methylation (i.e., mask the signal from target loci with buffy coat methylation above a certain threshold). However, masking of target loci with high background methylation proved to be an effective but blunt tool that warranted additional optimization. Described below are attempts to further improve the signal-to-background ratio using masking of target loci.

Attempts to improve signal-to-background ratio using masking of target loci included an analysis of 16 samples from 10 separate healthy subjects with the aim of identifying loci that when masked (filtered out) would reduce the signal-to-background ratio. The pan-cancer assay(s) described herein measured methylation at loci that are often hypermethylated in cancer. One limitation of the assay(s) was that not all methylation signal was cancer signal. This could have been due to loci that have non-zero amounts of background methylation (e.g., methylation in non-cancer subjects).

As noted above, limiting the amount of background methylation has an impact on accurate treatment monitoring. Without wishing to be bound by theory, methylation that is randomly presented can introduce spurious signal that may lead to incorrect clinical interpretations. Large amounts of background methylation (e.g., equal to or greater than 20 molecules) also raises the noise floor (e.g., the total amount of methylation seen in subjects without cancer). Large amounts of background methylation also effect the ability to detect relative changes of the cancer over time as the background is likely to be present each time point.

Previous work established an amplicon blacklist that contributed significant background methylation (see Example 2). This blacklist was generated by analyzing 4 samples from 4 healthy subjects.

Although effective at limiting the amount of background methylation, there remained a need for more comprehensive analysis of non-cancer samples to better establish an updated amplicon blacklist. As noted above, 16 specimens from 10 separate subjects previously run on the methylation assay were analyzed to determine an updated amplicon blacklist. The updated blacklist was applied to the methods described herein and samples were assessed for whether clinical interpretations changed significantly following analysis using the amplicon blacklist.

### Example Methods for Blacklist Determination

The training set included the 16 specimens from 10 separate subjects described above. Analysis included 8 specimens from batch 2 that did not have cancer but underwent a liver surgical procedure. These 8 specimens were collected from four subjects at 2 times. Subjects were 38, 39, 83 and 84 years old. Analysis also included 8 specimens from 6 healthy subjects: 2 subjects had 2 time points each, the rest each had one.

To determine an updated amplicon blacklist two approaches were used: a personalized amplicon blacklist that was patient or sample specific and a global amplicon blacklist.

### Personalized Blacklist

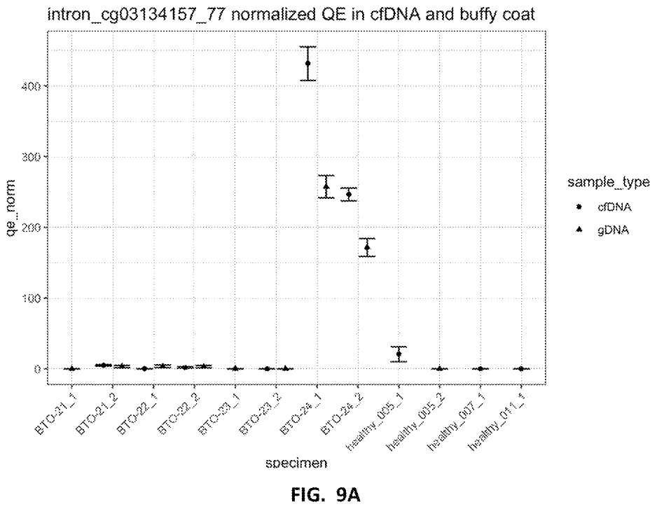

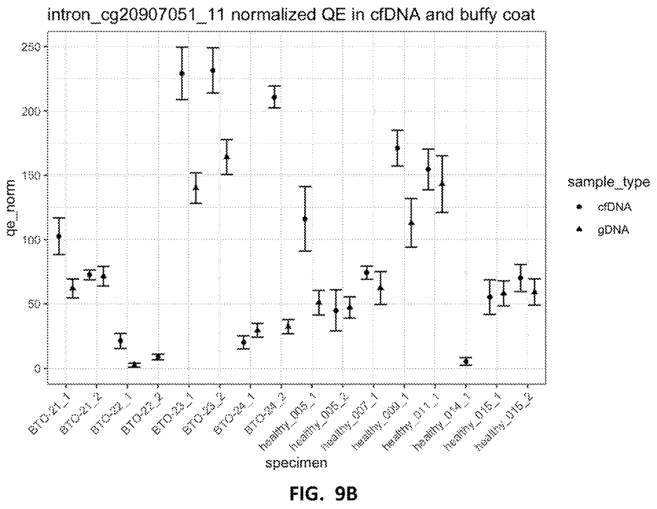

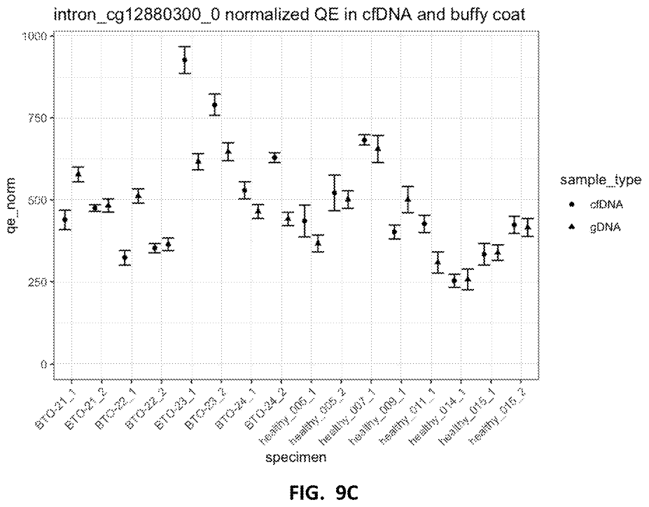

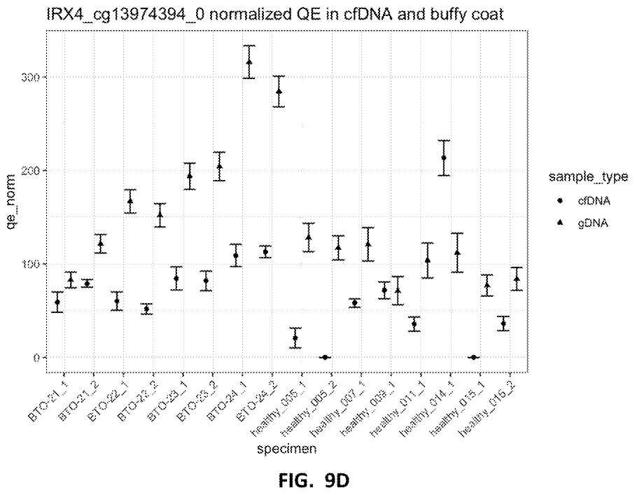

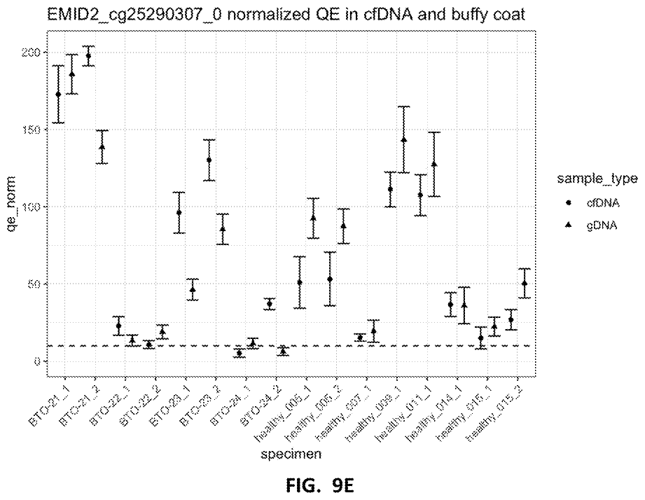

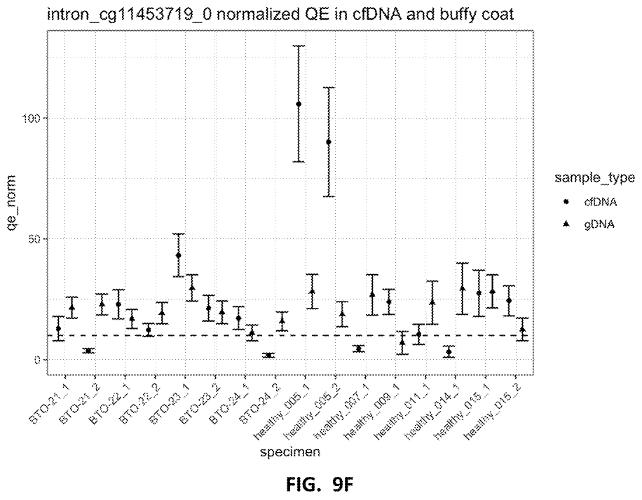

A personalized approach enables blacklisting of only loci determined likely to be contributing background methylation signal in a particular sample. One non-limiting example of “personalized blacklisting” or “sample-specific blacklisting” included evaluating the buffy coat methylation signal for loci with high methylation cfDNA signal in non-cancer subjects (see FIG. 9A-9F).

Additional factors for identifying loci to be included in “personalized blacklists” included determining age and sex of the patients from whom the samples were taken.

FIG. 9A shows an example of loci used in a “personalized blacklist”: intron cg03134157_77 (“intron 0313”). As shown in FIG. 9A, tumor QE for intron 0313 showed significantly greater cfDNA methylation than buffy coat methylation only when there as significant buffy coat methylation. Tumor QE was calculated by subtracting blue from red. Error bars indicated one standard deviation as estimated by molecular counting noise based on the raw QE. Overall, the consistency of methylation at intron 0313 or little to no methylation at intron 0313 meant that the buffy coat methylation threshold for this locus was about 20 tumor QE. This meant that the buffy coat methylation threshold would be about 20 tumor QE to properly exclude this locus. Note, an excessively small buffy coat threshold may risk blacklisting too many loci (essentially becoming a global blacklist).

A second loci was analyzed for its use in a “personalized blacklist.” FIG. 9B shows methylation of intron cg20907051_11 (“intron 2090”). In particular, intron 2090 showed non-significant differences in cfDNA methylation in buffy coat methylation regardless of whether there was significant buffy coat methylation (see FIG. 9B). Because the vast majority of healthy samples tested have non-zero tumor QE, intron 2090 is example of a locus to blacklist for all samples.

Additional loci analyzed for inclusion in a “personalized blacklist” are shown in FIG. 9C-9F. FIG. 9C shows that tumor QE for intron_cg12880300_0 had an average of 59 tumor QE and had greater than 2 tumor QE in 9 out of 16 specimens. This locus was an example of a loci to blacklist for all samples due to the consistently high buffy coat methylation and variable cfDNA methylation.

FIG. 9D shows tumor QE for locus IRX4_cg13974394_0 (“IRX4”). For the IRX4 locus, significant tumor QE (red greater than blue) was seen in just 1 out of 16 specimens. This one sample was not flagged during QC. In fact, reads per QCT were about 4.9 for this cfDNA sample, which was lower than the other samples but not significantly so. This data suggested that the IRX4 may be best assessed on a sample by sample basis. Additionally, the data suggested that the high gDNA signal makes any actual tumor QE measurement on this locus relatively imprecise. Moreover, methylation appears to be correlated with age in this locus. BTO-23 and BTO-24 are in their 80s, BTO-21 and BTO-22 are in their late 30s, and healthy volunteers are overwhelmingly on the young side.

FIGS. 9E-9F show tumor QE for locus EMID2_cg252903070 (“EMID2) and intron_cg1453719_0 respectively. As shown in FIG. 9E, a filter of 10 QE (dashed line) for gDNA would have filtered out BTO-21_2 BTO-23_1 and BTO-23_2 but not BTO-24_2. As shown in FIG. 9F, a filter of 10 QE (dashed line) for gDNA would have filtered out all sample except BTO-24_1 and healthy600961. In these two loci, a personalized blacklist approach would require a relatively strict threshold of 10 QE, but even that fails to ignore spurious methylation signals in several samples. Therefore, it may be best to include these loci on a global blacklist.

TABLE 2
Global blacklist
Top 20 amplicons based on mean tumor methylated QE

| amplicon_name | n_samples_gt2 | mean_tumor_norm | max_tumor_norm | mean_bg_norm |
|---|---|---|---|---|
| intron_cg12880300_0 | 9 | 59.175 | 309.998 | 276.094 |
| intron_cg20907051_11 | 12 | 35.4 | 178.619 | 51.837 |
| CD8A_cg00916536_0 | 13 | 21.457 | 66.402 | 47.985 |
| intron_cg03134157_77 | 3 | 16.839 | 173.932 | 26.812 |
| intron_cg11453719_0 | 7 | 12.72 | 77.711 | 7.741 |
| EMID2_cg25290307_0 | 5 | 12.093 | 58.953 | 18.124 |
| WT1_cg02524954_4 | 8 | 9.652 | 69.461 | 35.228 |
| intron_cg26763727_7 | 6 | 9.593 | 75.436 | 3.454 |
| TLX3_cg05787556_10 | 5 | 7.603 | 44.381 | 21.624 |
| IRX4_cg13974394_0 | 1 | 6.338 | 101.406 | 7 |
| C17orf64_cg12788108_85 | 7 | 6.044 | 46.582 | 0.388 |
| TNFRSF10C_cg05636175_0 | 10 | 5.538 | 42.451 | 0.119 |
| PRHOXNB_cg27513935_0 | 5 | 4.736 | 37.049 | 0.571 |
| ITPRIPL1_cg17932631_8 | 7 | 4.237 | 23.643 | 0 |
| CDH13_cg19369556_56 | 4 | 4.22 | 26.412 | 0.359 |
| ASCL1_cg17015844_34 | 8 | 3.897 | 28.098 | 0.157 |
| NPY5R_cg11784623_3 | 5 | 3.508 | 39.096 | 1.196 |
| BARHL1_cg19793376_2 | 5 | 3.155 | 30.034 | 0.491 |
| intron_cg15992563_52 | 3 | 2.877 | 30.529 | 0.143 |
| DIDO1_cg24804517_46 | 9 | 2.838 | 11.782 | 0.238 |

To determine the criteria for including amplicons on a global blacklist, an empirical approach was used with set thresholds for mean tumor methylated QE and max tumor methylated QE (described below). The thresholds were applied to determine the blacklist and calculate the new total tumor QE in non-cancer samples. Thresholds were also applied to previous analysis (e.g., UCSD and UF results) to determine if these results were still clinically valid.

This analysis focused, in part, on the top 20 amplicons based on mean tumor methylated QE (see Table 2). In Table 2, the columns indicate the number of samples with greater than 2 tumor QE for that locus (n_samples_gt2), mean tumor QE (mean_tumor_norm) and max tumor QE (max_tumor_norm) across all 16 samples, and the mean buffy coat methylated QE (mean bg_norm).

Based in part on the analysis of the top 20 amplicons as shown in Table 2, the thresholds for the global blacklist included: (1) max tumor QE in any non-cancer specimen >15; and (2) mean tumor QE (including 0s)>2.

In a non-limiting example, applying the thresholds of (1) max tumor QE in any non-cancer specimen >15; and (2) mean tumor QE (including 0s)>2 resulted in a global blacklist that included 32 amplicons. Compared to the previous blacklist (see Table 3), 16 amplicons were overlapping, which meant 6 amplicons were removed and 16 were added.

TABLE 3

| amplicon_name | old_blacklist | new_blacklist |
|---|---|---|
| ALDH1A2_cg02900766_3 | yes | yes |
| ASCL1_cg17015844_34 | yes | yes |
| BARHL1_cg19793376_2 | | yes |
| C17orf64_cg12788108_85 | yes | yes |
| CD8A_cg00916536_0 | yes | yes |
| CDH13_cg19369556_56 | yes | yes |
| DIDO1_cg24804517_46 | yes | yes |
| EMID2_cg25290307_0 | | yes |
| FOXF2_cg19519310_22 | | yes |
| GDF10_cg14720763_6 | | yes |
| GPC5_cg26960333_0 | yes | yes |
| HAAO_cg20857709_0 | yes | yes |
| HIST1H2BB_cg26426142_0 | | |
| intron_cg01418261_0 | | yes |
| intron_cg01683794_56 | yes | yes |
| intron_cg03134157_77 | | |
| intron_cg03933990_11 | yes | yes |
| intron_cg04908789_0 | | yes |
| intron_cg11453719_0 | yes | yes |
| intron_cg12880300_0 | yes | yes |
| intron_cg15992563_52 | | yes |
| intron_cg16476975_10 | yes | |
| intron_cg20907051_11 | yes | yes |
| intron_cg26763727_7 | yes | yes |
| intron_cg27452217_0 | | |
| IRX4_cg13974394_0 | yes | yes |
| ITPRIPL1_cg17932631_8 | yes | yes |
| JAM3_cg04913265_0 | yes | yes |
| KCNA6_cg10671668_0 | | yes |
| NPY5R_cg11784623_3 | | yes |
| OTX2OS1_cg14248715_9 | yes | |
| PRHOXNB_cg27513935_0 | yes | yes |
| TLX3_cg05787556_10 | yes | yes |
| TLX3_cg25942450_0 | yes | |
| TNFRSF10C_cg05636175_0 | | yes |
| USP44_cg00927554_0 | | yes |
| WT1_cg02524954_4 | | yes |
| ZFP28_cg12973930_0 | | yes |
| total | 22 | 32 |

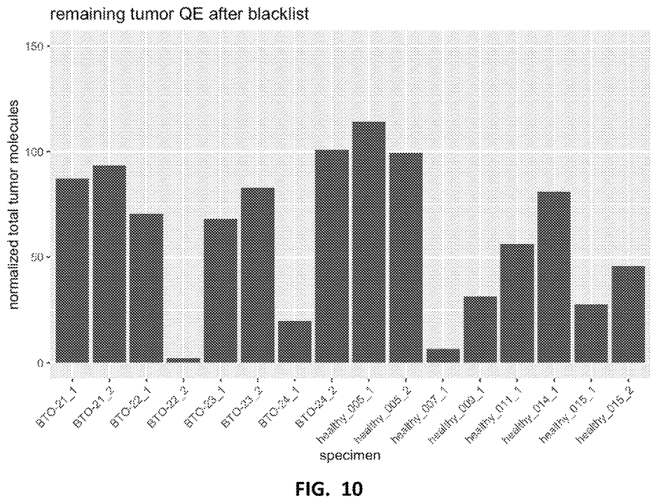

The new global blacklist generated based on the thresholds described above (i.e., (1) max tumor QE in any non-cancer specimen >15; and (2) mean tumor QE (including 0s)>2) was applied to 16 non-cancer specimens to determine the noise floor. As shown in FIG. 10, no sample had methylation greater than 114 tumor QE. Therefore, the noise floor was set at 120 for additional analysis. However, one skilled in the art would appreciate the noise floor would change based on a similar type of analysis applied to a different sample or different set of samples.

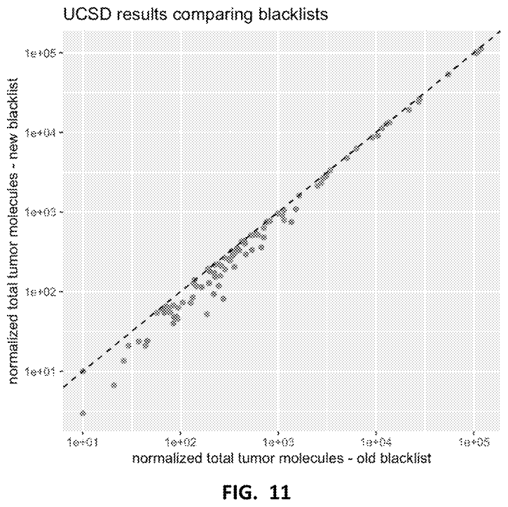

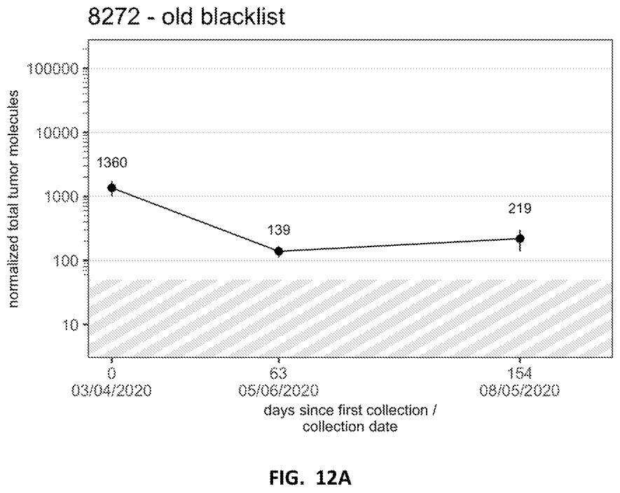

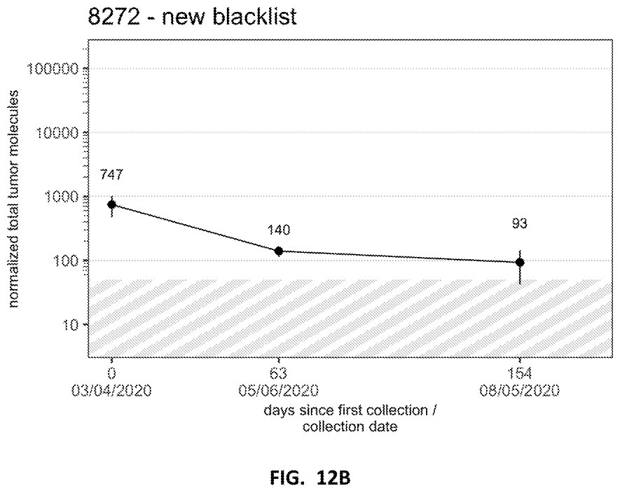

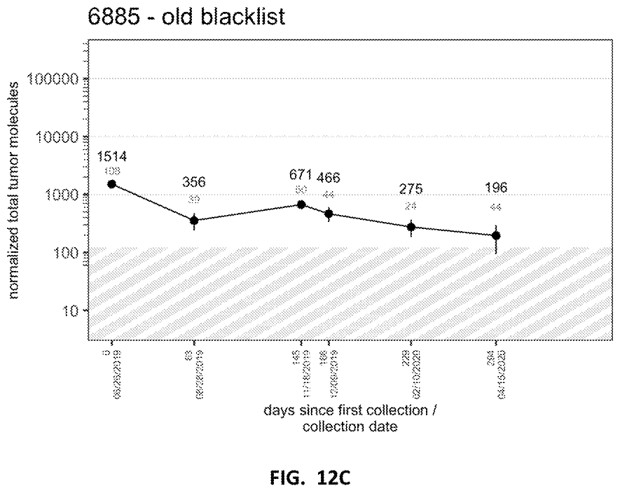

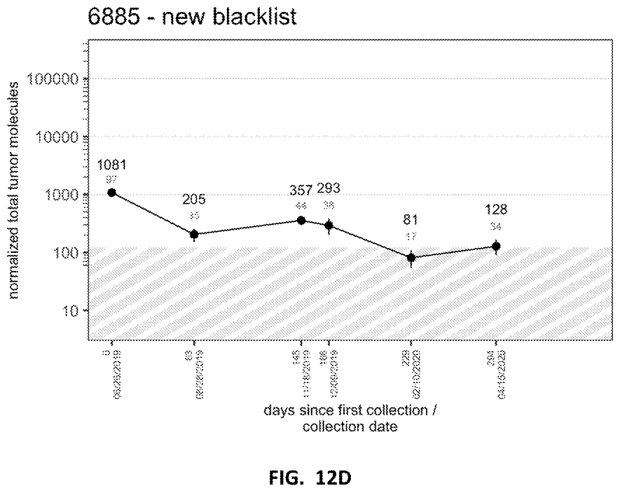

The new global blacklists were then compared to the old global blacklists. As shown in FIG. 11, comparing the normalized total tumor molecules using either the old or new global blacklist, most subjects had equal if not fewer tumor molecules. In addition, the methylation results for all 30 subjects were manually inspected and assessed whether the clinical interpretations would have changed. Out of 30 total subjects, only 2 subjects had slightly different results, both of which were within the statistical noise. For both subjects 8272 and 6885, the results for the last collection changed slightly, but still within molecular noise error bars (see FIGS. 12A-12D). Overall, this data showed that small changes to the blacklist did not significantly affect the clinical results.

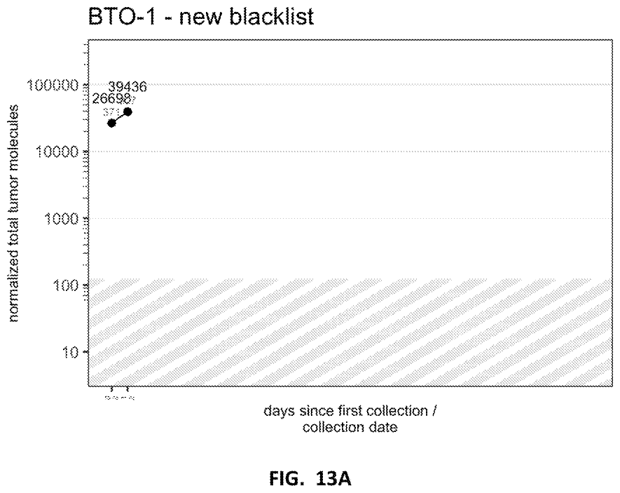

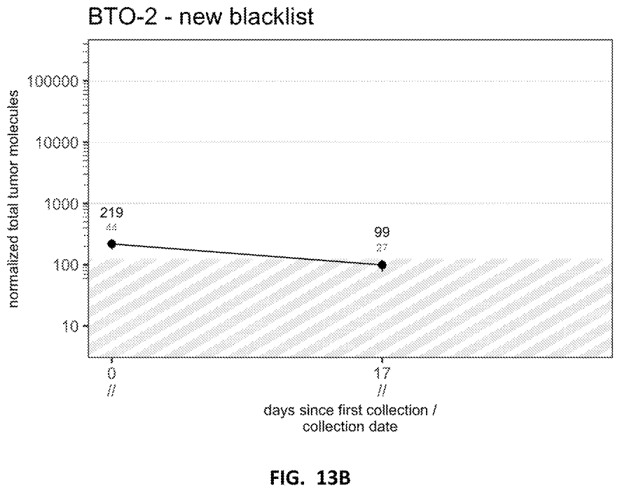

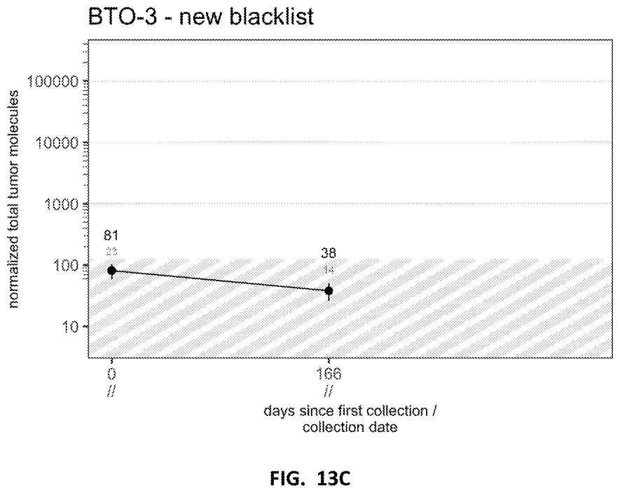

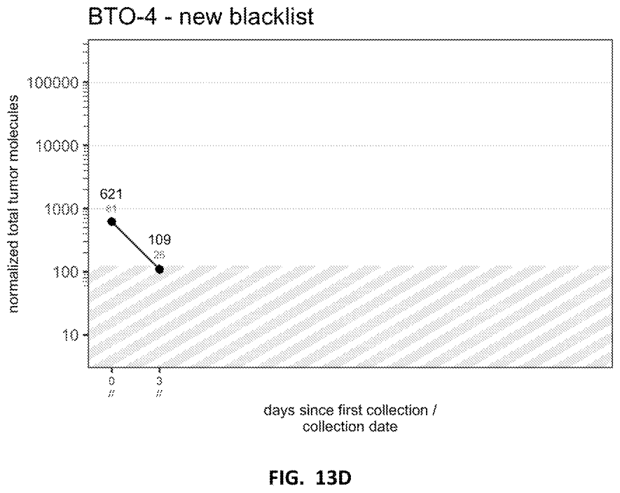

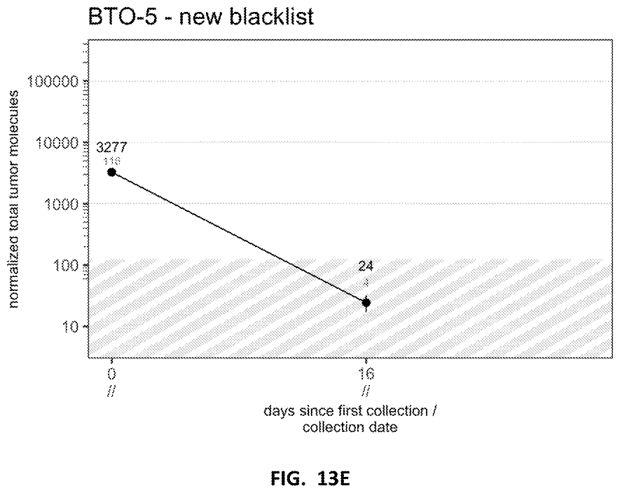

The new global blacklists were then compared to the old global blacklists for 5 patient samples (see BTO-1 to BTO-5 in FIGS. 13A-13E, respectively) that had surgical removal of liver cancer, though the patients had a range of cancer stages. As shown in FIGS. 13A-13E, the trend for each patient was concordant with the previous analysis. Additionally, each patient had a decrease in tumor methylation, with the exception of BTO-1, who was a Stage 4 liver cancer patient where high levels of methylation was detected and a sample was provided one day after surgery.

### CONCLUSION

This data showed the benefits of a global blacklist versus a personalized blacklist (either patient- or sample-specific). In particular, a global blacklist avoided having to deal with unanswered biological variability and temporal variability questions. However, this analysis does not rule out use of a personalized blacklist, but additional optimization may be needed to design effective personalized blacklists.

This data also showed that adjusting the blacklist from 22 to 32 amplicons did not significantly change clinical results beyond measurement noise in two clinical study datasets. Importantly, the noise floor increased from 50 to 120 normalized total tumor QE.

### Example 7. Analytical and Clinical Validation of the Pan-Cancer Assay

The pan-cancer assay provided in this disclosure and described, for example, in Example 5 underwent analytical validation, including testing for accuracy, precision, reproducibility, sensitivity, and specificity.

### INTRODUCTION AND SUMMARY OF VALIDATION ARMS

Accurate, rapid, and accessible treatment monitoring for cancer patients is an unmet medical need. When the outcome of a cancer treatment regimen is uncertain, determining whether a treatment is effective for a patient earlier rather than later could enable a switch to a different treatment regimen thus potentially prolonging life, reducing unnecessary side effects from ineffective treatments and improving quality of life, and improving the overall efficiency of the health care system. Assessing treatment efficacy earlier, or even predicting the eventual treatment outcome, can be of extra importance for late stage cancer patients or for the efficient execution of clinical studies for novel cancer therapies, where time is of the essence.

ctDNA (circulating tumor DNA), obtained through a liquid biopsy from the cancer patient, has been shown to reflect the amount of cancer present in the patient. In addition, methylation has been shown to be a robust biomarker of cancer, with several groups developing methylation-based assays using ctDNA for various cancer diagnostics applications. Using our patented QCT technology (Tsao et al., 2019), Applicant's has developed a novel assay called pan cancer assay to quantify the amount of methylation in the ctDNA for an accurate and precise treatment monitoring application.

The validation studies were conducted to demonstrate the analytical and clinical validity of the pan-cancer treatment monitoring assay.

The analytical validations included: Arm 1: Accuracy, Precision, and Reproducibility on Sheared Tumor DNA Samples; Arm 2: Concordance on Clinical Samples with Known Clinical Outcomes; Arm 3: Limit of Detection using Sheared Tumor DNA Samples; and Arm 4: Diversity of Cancer Types.

### Arm 1: Accuracy, Precision, and Reproducibility on Sheared Tumor DNA Samples

The aim for Arm 1 was to calculate the accuracy, precision, and reproducibility based on sheared tumor DNA samples. Replicates of sheared tumor DNA samples at 1% and 2% tumor fraction were made from a total of 7 different tumors from 7 different cancer types. The sheared tumor DNA samples were processed in two different batches, and loaded on two different sequencers. The Response Score, which describes the amount of methylation in the sample, was calculated for each sample, and the Response Score for 1% and 2% tumor fraction samples were compared with each other.

In summary and described in further detail below, data from Arm 1 showed: 100% accuracy (i.e., 40 out of 40 comparisons called correctly); 100% precision (i.e., 0 subjects with discordant results within each batch); and 100% reproducibility (i.e., 9 out of 19 comparisons concordant between batches).

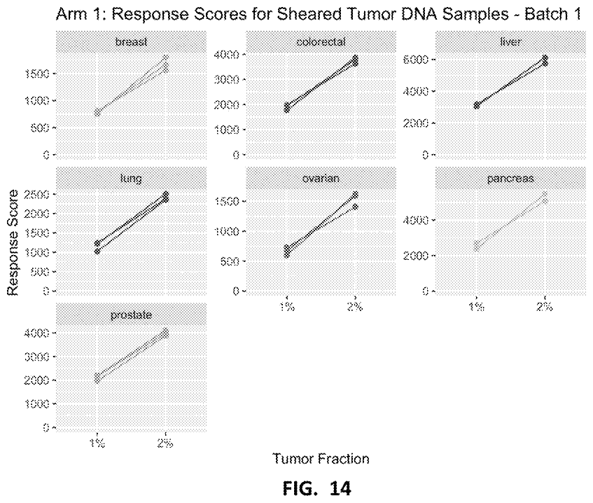

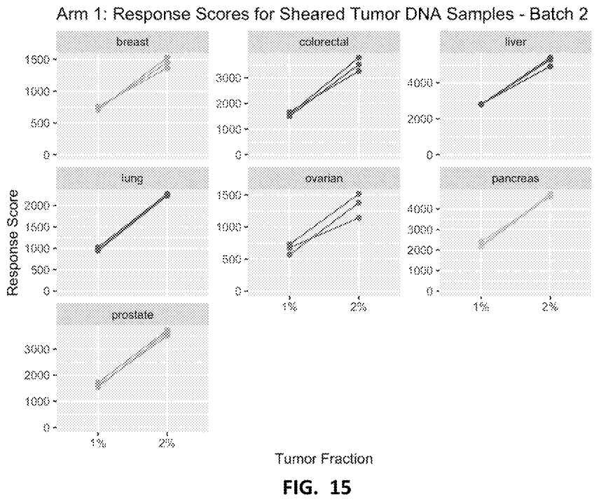

In both batches, all comparisons between 1% and 2% tumor fraction samples that passed QC were called as “Increase,” which is concordant with the identity of these samples. The Response Scores from both batches are as shown in FIG. 14 and FIG. 15.

Additional calculations were made for sensitivity and specificity of the pan-cancer assay. Sensitivity was calculated based on comparing 1% and 2% samples, as described above, and specificity was calculated by comparing 1% samples against themselves and 2% samples against themselves within each batch. This analysis revealed sensitivity=100% [95% CI: 91.2%, 100%](i.e., 40 out of 40 comparisons) and specificity=100% [95% CI: 95.5%, 100%](i.e., 80 out of 80 comparisons).

Overall, the result from Arm 1 demonstrates that the pan-cancer assay is accurate, precise, and reproducible across operators and sequencers.

Arm 2: Concordance on Clinical Samples with Known Clinical Outcomes

The aim for Arm 2 was to assess the accuracy of the pan-cancer assay on clinical samples from a total of 20 subjects. These 20 subjects were composed of 8 cancer subjects with known clinical outcomes, and 12 healthy subjects without known history of cancer. Response Scores were measured at two time points for each subject. Calls were made based on the Response Scores from both time points, and the calls were compared with known clinical outcomes.

In summary and described in further detail below, data from Arm 2 showed: 100% accuracy for cancer subjects (6 out of 6 concordant calls); 100% accuracy for healthy subjects (11 out of 11 concordant calls).

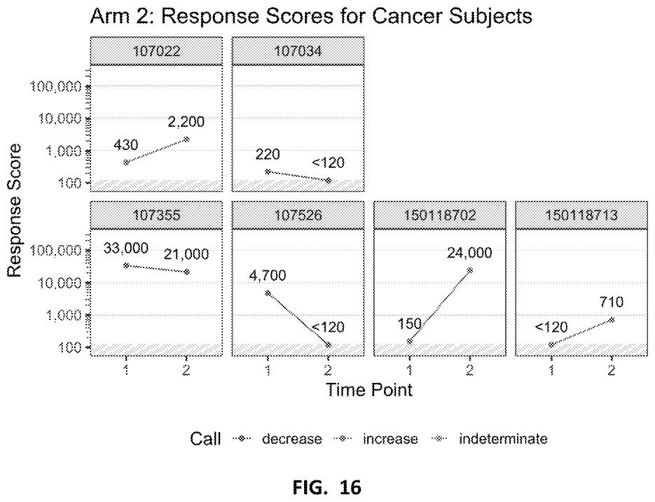

Out of the 6 cancer subjects with detectable signal, all 6 subjects had calls that were concordant with known clinical outcomes. Results for all cancer subjects are plotted in FIG. 16, and known clinical outcomes can be found in Table 4.

TABLE 4
Known clinical outcomes for 6 cancer subjects

| Subject | Cance | Known Clinical | Validation | |
|---|---|---|---|---|
| ID | Type | Outcome | Call | Interpretation |
| 107034 | Lung | Partial response | Decrease | Concordant |
| 107355 | Lung | Partial response | Decrease | Concordant |
| 107022 | Pancreas | Progression | Increase | Concordant |
| 150118702 | Pancreas | Progression | Increase | Concordant |
| 150118713 | Lung | Progression | Increase | Concordant |
| 107526 | Lung | Partial response | Decrease | Concordant |

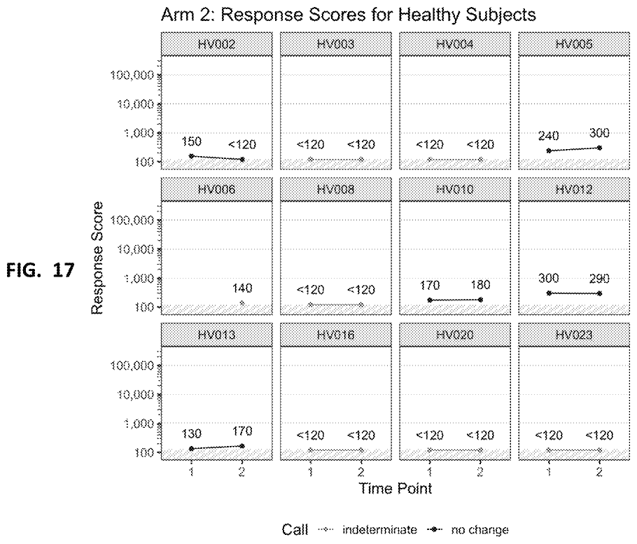

Out of the 12 healthy subjects, 11 subjects had no change in cancer or an Indeterminate call, which is concordant with the healthy status of these subjects (see FIG. 17).

In summary, the results for Arm 2 demonstrate the validity of the pan-cancer assay to accurately assess clinical samples from a total of 20 subjects.

### Arm 3: Limit of Detection Using Sheared Tumor DNA Samples

The aim for Arm 3 was to assess the limit of detection of the pan-cancer assay by measuring sensitivity at different input conditions.

For these experiments, the sheared tumor DNA samples at 0%, 0.25%, 0.5%, 1%, and 2% tumor fraction were made from a single tumor and its matching buffy coat and analyzed using the pan-cancer assay. For each tumor fraction, the Response Score was measured for 16 replicates, and these measurements were used to calculate CV. Assay sensitivity was calculated by comparing Response Scores for samples at each tumor fraction with the Response Scores of 0% samples, from which the limit of detection was determined.

In summary and described in further detail below, the results showed the Limit of Detection=0.25% Tumor Fraction.

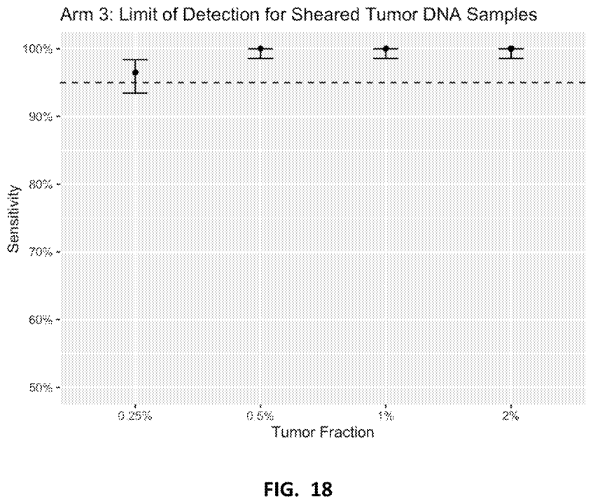

The limit of detection was defined as the lowest tumor fraction with at least 95% sensitivity. For 0.25% tumor fraction samples, the sensitivity was 96.5% [95% CI: 93.4%, 98.4%]. The sensitivities for each tumor fraction are shown in FIG. 18.

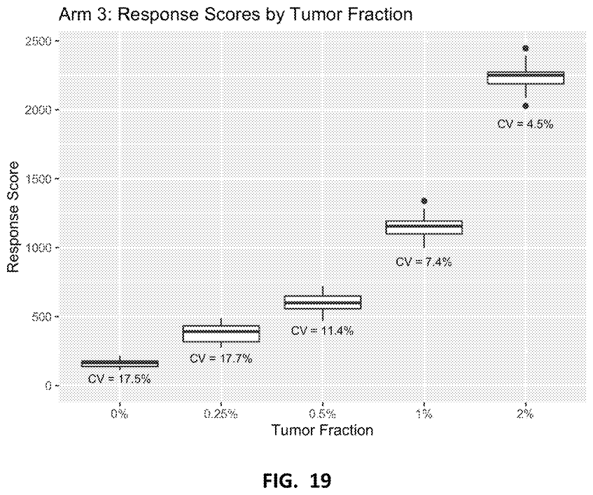

The Response Scores for each tumor fraction are plotted in FIG. 19. As expected, higher tumor fractions corresponded to higher Response Scores. Within each group of technical replicates, the CV of the Response Score was calculated for a given tumor fraction (see FIG. 19). There is clear separation between the 0% and 0.25% tumor fractions, which is in line with the 0.25% tumor fraction limit of detection (see FIG. 19). The standard deviation and mean of each tumor fraction were used to calculate CV.

In summary, the results for Arm 3 demonstrate that the limit of detection for the pan-cancer assay is at least 0.25% tumor fraction.

### Arm 4: Diversity of Cancer Types

The aim for Arm 4 is to assess the performance of the pan-cancer assay on different cancer types. For each of the 54 different cancer patients spanning 10 unique cancer types, a tumor gDNA sample and a matched buffy coat gDNA sample were processed.

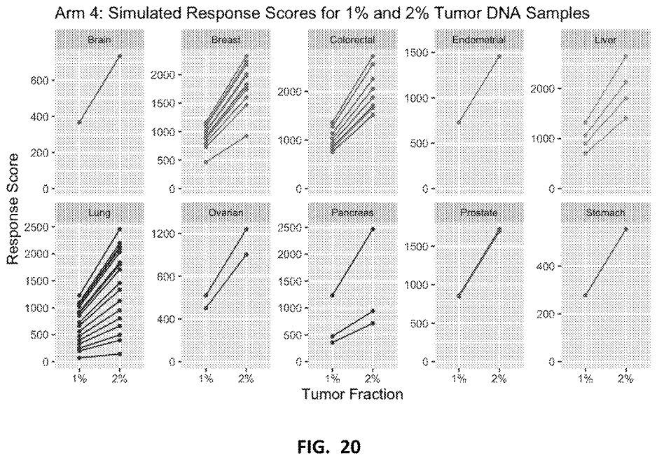

For these experiments, bioinformatic simulations were used to assess the performance of the pan-cancer assay on different cancer types. For example, bioinformatics was used to simulate 1% and 2% tumor DNA samples by scaling down the number of methylated molecules based on estimated tumor purity. Using the standard analysis workflow, all 2% tumor sample Response Scores were correctly called as Increased relative to 1% tumor sample Response Scores (see FIG. 20; Table 5). As shown in Table 5, sensitivity across cancer types was 100% [95% CI: 93.4%, 100%].

TABLE 5
Sensitivity for Distinguishing 1% and 2%
Tumor DNA Samples Across Cancer Types

| Cancer | Fraction of Cancer Patients | Sensitivity [95% Confidence |
|---|---|---|
| Type | Called Correctly | Interval] |
| Lung | 18 out of 18 | 100% [81.5%, 100%] |
| Colorectal | 11 out of 11 | 100% [71.5%, 100%] |
| Breast | 11 out of 11 | 100% [71.5%, 100%] |
| Liver | 4 out of 4 | 100% [39.8%, 100%] |
| Pancreas | 3 out of 3 | 100% [29.2%, 100%] |
| Ovarian | 2 out of 2 | 100% [15.8%, 100%] |
| Prostate | 2 out of 2 | 100% [15.8%, 100%] |
| Endometrial | 1 out of 1 | 100% [2.5%, 100%] |
| Stomach | 1 out of 1 | 100% [2.5%, 100%] |
| Brain | 1 out of 1 | 100% [2.5%, 100%] |
| TOTAL | 54 out of 54 | 100% [93.4%, 100%] |

In summary, this data confirms that the pan-cancer assay performs with high sensitivity across a variety of cancer types. For all 54 cancer patients included in this validation arm, the pan-cancer assay detected increases in Response Score when a change from 1% to 2% tumor fraction was simulated.

### CONCLUSION

This analytical and clinical validation report describes the results from the experimental arms performed to support the validation of the pan-cancer assay. Overall, the results show that the assay has high accuracy and precision, high reproducibility, high sensitivity and specificity, and a low limit of detection. By measuring the amount of ctDNA in cancer patients through a liquid biopsy, the pan-cancer assay gives oncologists additional information that can be used to improve clinical outcomes for their cancer patients.

### Example 8. Detection of Methylation Concordant with Disease Progression

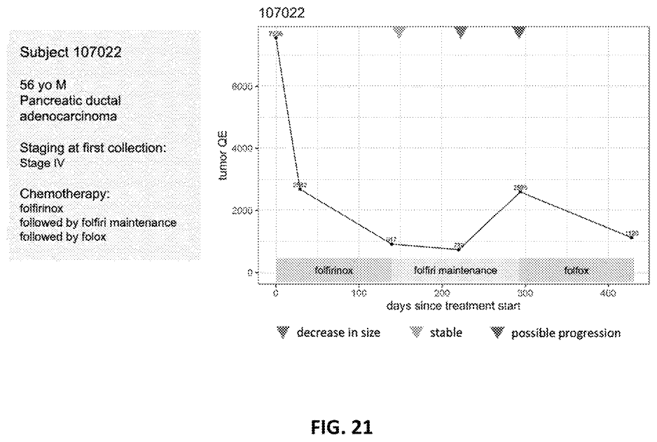

The pan-cancer assay was used to assess concordance between methylation and disease progression. For this analysis, methylation and CT imaging were both used to assess disease progression in four patients. Methylation was monitored using the pan-cancer assay. CT imaging was performed by the clinician and used to stage disease progression. The data as shown in FIGS. 21-24 is presented as methylation (“tumor QE”) over time (“days since treatment start”) with triangles indicated a clinician-based staging of disease progression.

FIG. 21 shows methylation analysis for a 56 year old male subject with pancreatic ductal adenocarcinoma stage IV at time of first collection and clinician-based staging of disease progression (see triangles in FIG. 21). As noted in FIG. 21, methylation analysis was performed at the indicated time points, which coincided with a treatment regimen of: folfirinox (“folfiri”), followed by folfiri maintenance, followed by folox. Here, methylation level was in concordance with the clinician-based staging. For example, a decrease in methylation corresponded to a decrease in size of the tumor or stable growth of the tumor (as indicated by the clinician), and an increase in methylation corresponded with possible disease progression (as indicated by the clinician).

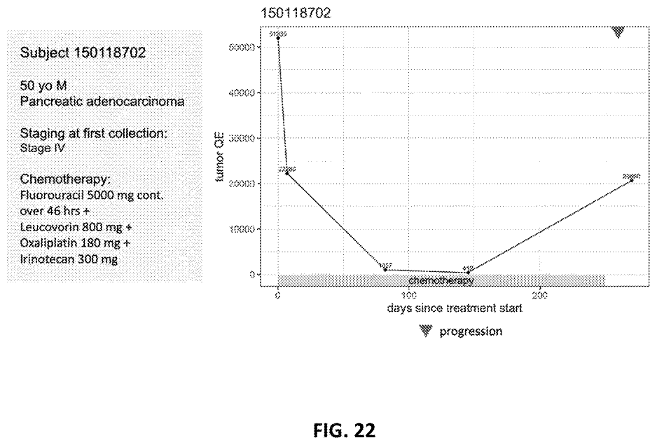

FIG. 22 shows methylation analysis for a 50 year old male subject with pancreatic adenocarcinoma stage IV at time of first collection and clinician-based staging of disease progression (see triangle in FIG. 22). As noted in FIG. 22, methylation analysis was performed at the indicated time points, which coincided with a treatment regimen of: fluorouracil 5000 mg continuous over 46 hours+leucovorin 800 mg+oxaliplatin 180 mg+Irinotecan 300 mg. Here, the methylation level was in concordance with the clinician-based staging. For example, as methylation levels increased prior to day 200 and continuing to rise beyond day 200, the clinician-based staging indicated disease progression.

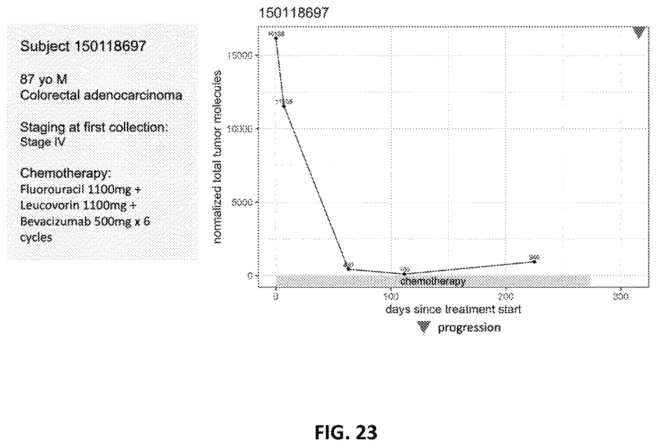

FIG. 23 shows methylation analysis for an 87 year old male subject with colorectal adenocarcinoma stage IV at time of first collection. FIG. 23 also shows clinician-based staging of disease progression (see triangle in FIG. 23). As noted in FIG. 23, methylation analysis was performed at the indicated time points, which coincided with a treatment regimen of: 6 cycles of fluorouracil 1100 mg+leucovorin 1100 mg+Bevacizumab 500 mg. Here, the methylation level was in concordance with the clinician-based staging. For example, as methylation levels increased prior to day 200 and continuing to rise beyond day 200, the clinician-based staging indicated disease progression.

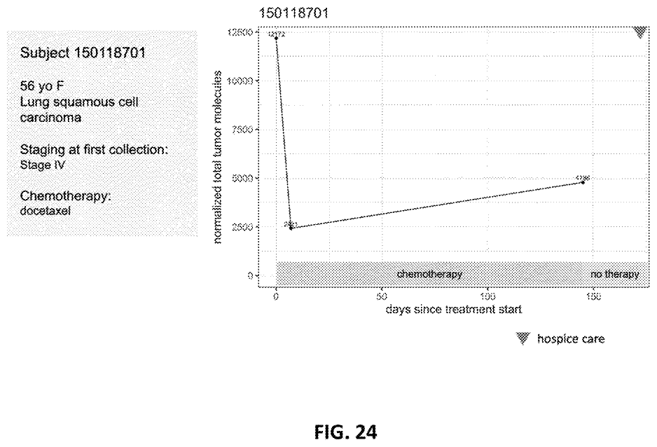

FIG. 24 shows methylation analysis for a 56 year old female subject with lung squamous cell carcinoma stage IV at time of first collection. FIG. 24 also shows clinician-based staging of disease progression (see triangle in FIG. 24). As noted in FIG. 24, methylation analysis was performed at the indicated time points, which coincided with a treatment regimen of: docetaxel. Similar to the patients analyzed in FIGS. 21-23, the methylation level here was in concordance with the clinician-based staging.

### Example 9. Concordance with Variant Allele Fraction

The pan-cancer assay was also used to assess concordance between methylation and variant allele fraction.

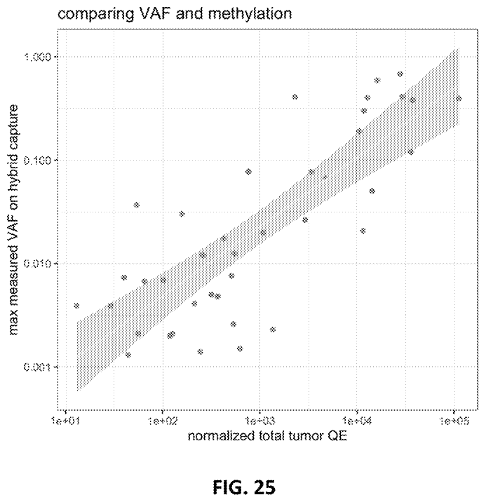

For this experiment, 40 samples were run through a treatment selection assay. For each sample, an aliquot of plasma was collected at the same time point as the methylation assay. From the treatment selection assay, the maximum variant allele fraction (VAF) was calculated and compared against the methylation assay. As shown in FIG. 25, there was a correlation between the maximum VAF and the number of methylated molecules. A perfect one-to-one correlation was not expected because somatic mutations were likely to be heterogenous throughout the tumor and may not reflect the total abundance of the cancer.

One limitation of treatment selection assays was false negative actionable mutations. Given the correlation between VAF and methylation, the methylation results were used to inform whether a false negative actionable mutation identified with VAF was likely a false negative, or not. For example, if no actionable mutations were found but methylation levels were low, this suggested a low tumor fraction at this time point and possible false negative actionable mutations due to the low number of tumor molecules. On the other hand, if no actionable mutations were detected but methylation levels were high, this suggested a high tumor fraction at this time point, and that a false negative actionable mutation of large magnitude would be unlikely.

In summary, the concordance between VAF and methylation showed that methylation (i.e., methylation analyzed and identified according to the methods described herein) can be used to inform the results of VAF analysis, which in turn, can be used for supplementing therapy selection assay decisions.

### Example 10. Changes in Methylation Profiles

The pan-cancer assay was also used to assess methylation profiles over time. For these experiments, methylation patterns were analyzed for two subjects (Subject 6885 and subject 5458) over 294 days and 250 days, respectively. In summary and described in greater detail below, it was observed that some subjects had relatively constant methylation profiles over time, whereas some patients had large changes in their methylation profiles.

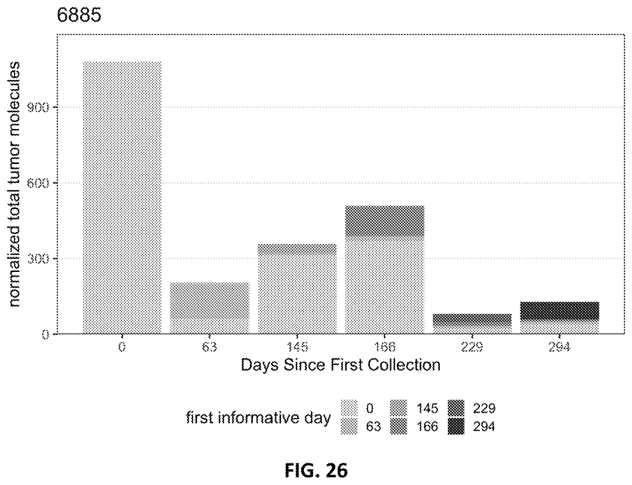

For example, as shown in FIG. 26, for Subject 6885, each new time point had novel loci that were methylated. However, with the exception of loci present at the Day 0 time point, these novel loci did not contribute a significant amount of methylation at subsequent time points. For example, the loci that first appear on Day 63 did not contribute a significant amount of methylation at later time points.

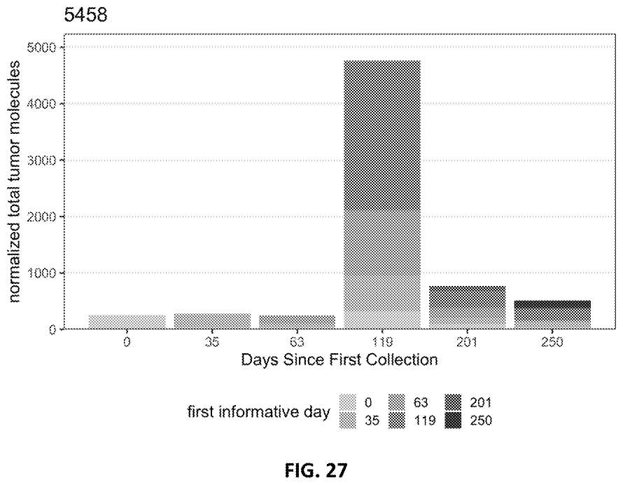

In a second, non-limiting example shown in FIG. 27, for Subject 5458, there was an increase in total methylation on Day 119 that combined with interesting methylation patterns. In particular, on Day 119, slightly more than half of the methylation comes from new loci (i.e., methylated loci not detected at day 0, 35, or 63), suggesting that the tumor significantly evolved compared to previous time points. In fact, time points after Day 119 maintained a significant fraction of loci that first appeared on Day 119, indicating that the change in tumor methylation was persistent. Based on the large increase and the sudden change in composition of the methylation pattern, this subject was flagged as warranting a repeat of a treatment selection assay to detect any novel somatic mutations present in the cancer. Clinically, a pattern such as that seen in Subject 5458 may indicate the need to re-evaluate treatment options.

### CONCLUSION FOR EXAMPLES 8-10

Overall, this data established the pan-cancer assays utility for (1) detecting concordance between methylation and disease progress; (2) detecting concordance between methylation and variant allele fraction; and (3) assessing methylation profiles over time.

### Example 11: Methylation Pattern in Patient with Pancreatic Ductal Adenocarcinoma

The pan-cancer assay was used to assess methylation profiles over time in a patient with pancreatic ductal adenocarcinoma. The assay was used to detect a decrease in methylated molecules in the circulating tumor DNA (ctDNA) compared to the initial measurement. Aberrantly methylated DNA is a known marker of cancer cells (PMID 15542813), and a change in methylated ctDNA corresponds to a change in tumor fraction. This result suggests that tumor fraction has decreased compared to the previous measurement.

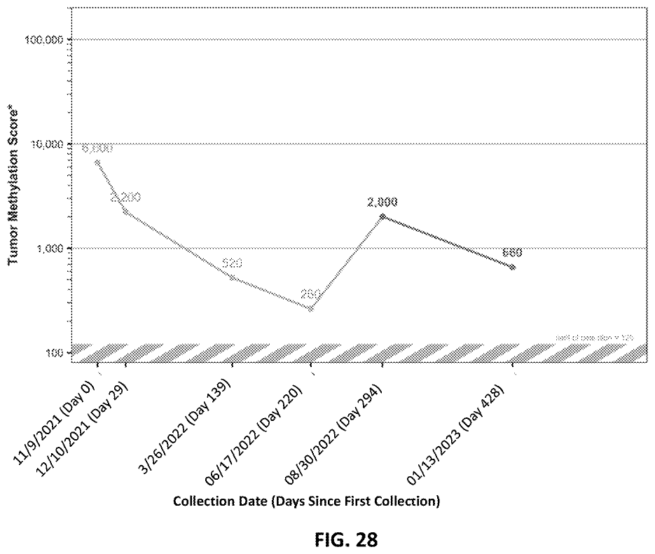

For these experiments, plasma and buffy coat were isolated from whole blood collected in a Streck cell-free DNA tube. Cell-free DNA (cfDNA) was extracted from the plasma, and genomic DNA (gDNA) was extracted from the buffy coat. The number of methylated molecules was quantified in both cfDNA and gDNA using QCT molecular counting technology (PMID: 31591409) at >500 locations in the genome known to be hypermethylated in cancer compared to non-cancerous tissue and blood. Methylation measured in gDNA was subtracted from cfDNA methylation in order to remove background from the ctDNA signal. The remaining cfDNA methylated molecules were summed across all hypermethylation locations to calculate the Tumor Methylation Score™. FIG. 28 shows the Tumor Methylation Score™ represented as the normalized sum of methylated molecules at >500 loci that are hypermethylated in circulating tumor DNA (ctDNA). As shown in FIG. 28, a decrease in Tumor Methylation Score™ was detected that corresponds to a decrease in methylated ctDNA molecules. In particular, FIG. 28 shows that tumor fraction decreased compared to the previous measurement by about 3.1-fold.

The Tumor Methylation Score™ from the current collection was compared to the most recently reported Tumor Methylation Score™ to determine an increase, decrease, or no change call. The change in Tumor Methylation Score™ must exceed a significance threshold in order to be reported as an increase or a decrease. No interpretive calls for change in Tumor Methylation Score™ are made for baseline tests without any prior collections. Results should be discussed with a medical professional and interpreted in conjunction with the patient's complete clinical history within the context of multiple timepoints.

In some cases, methylation may not be reported when the sample contains an insufficient amount of DNA. Results below the Tumor Methylation Score™ limit of detection (LOD), depicted on the graph by cross-hatched shading, are not interpreted and will be reported as less than the LOD. Performance specifications based on internal validation studies demonstrated that this assay can distinguish a 0.2 percentage point change in tumor fraction with 3 standard deviations of separation. This pan-cancer assay was designed for quantifying Tumor Methylation Score™ in patients with solid tumors. Results may vary or be invalid if the patient has undergone recent blood transfusion, stem cell transplant, or other procedures that may significantly affect the composition of cfDNA or buffy coat gDNA.

### Example 12. cfDNA Methylated Tumor DNA Measurements with Plasma VAF-Based Tumor Fraction Estimates

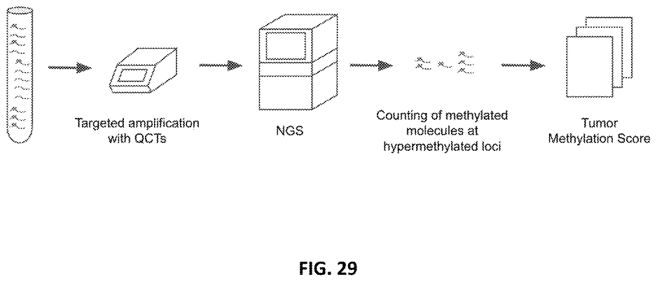

A study was conducted to evaluate correlation of VAF with Tumor Methylation Score (TMS) and Tumor Fraction (TF) in a pan-cancer cohort of clinical samples. The Northstar Response® assay was used to generate TMSs and TFs, while the Northstar Select® assay was used to identify somatic variants and CHIP mutations. Northstar Response® is a tumor-naive, cfDNA methylation-based therapy response monitoring assay that measures methylated molecules by counting over 500 targeted loci in plasma and buffy samples. When possible, multiple buffy samples from the same patient were pooled in silico to improve non-tumor cfDNA measurement confidence intervals. FIG. 29 provides an overview of the Northstar Response® assay. Hypermethylation is indicated by rectangles on cfDNA fragments. QCTs are co-amplified along with cfDNA, followed by next-generation sequencing, which is used to calculate the methylated reads per locus. Buffy genomic DNA (gDNA) follows the same protocol and is used to control for cfDNA contributions from circulatory immune cells. Northstar Select® is a tumor-naive cfDNA based assay which detects clinically actionable somatic variants with a high degree of sensitivity and specificity.

### Methods

### Tumor Fraction Estimation

Tumor Fraction (TF), the proportion of cfDNA fragments corresponding to ctDNA, was computed by fitting a zero-truncated Poisson (ZTP) distribution to NSR methylated read counts. The estimated Poisson mean was normalized to coverage via control probes corresponding to ubiquitously methylated loci, yielding the plasma-only TF. A ZTP-based based approach was used to integrate gDNA as a control, subtracting the gDNA estimate from the cfDNA estimate for normalization, yielding the TF.

To analytically measure TF performance, contrived tumor samples were serially diluted alongside a negative control. 16 experimental replicates for plasma, and 16 for buffy, were run per each concentration. The estimated number of methylated loci was derived from the ZTP distribution by including an estimated number of probe non-detection events given the tumor fraction and methylated loci.

### Analysis

For correlation analysis on a per-subject level, subjects were required to have three or more distinct samples, with each sample representing a NSR TF estimate and NSS VAF summary statistic. To ensure measurable comparisons, positive detection was require in both NSR and NSS. Summary statistics were computed for both NSR (TF; TMS) and NSS (max(VAF); sum(VAF)) detection methods. Aggregate metrics such as max(VAF) or sum(VAF) required VAF<40% to eliminate germline variants. Northstar Select CHIP is an orthogonal assay which detects variants in CHIP-prone genes with buffy coat gDNA. CHIP calls, positive or negative, were integrated into comparisons.

### Results

Table 6 shows demographics for a NSS-NSR correlation cohort by subject. Correlation and R² between NSS and NSR was estimated for subjects with at least three tumor fraction measurements. The cohort was derived from clinical study and non-study samples completed on NSS and NSR.

TABLE 6
Details of Cohort.

| Characteristic | N = 196¹ |
|---|---|
| Sex | |
| Female | 101 |
| Male | 95 |
| Age | 63 (13) |
| Primary Tumor Organ | |
| Breast | 38 |
| Colon/Rectum/Anus | 37 |
| Pancreas | 30 |
| Stomach/Esophagus | 26 |
| Lung | 20 |
| Other | 20 |
| Prostate | 12 |
| Ovary | 6 |
| Kidney | 4 |
| Skin | 2 |
| Brain | 1 |
| Number of Timepoints | 4.03 (1.61) |

¹n; Mean (SD)

Table 7 shows the median R² between NSS variant detection summary statistics across all variants (mean; median; sum; max) and NSR measurements(TMS; TF), per each subject. The highest R² were observed with the sumo function and TMS. When eliminating variants of undetermined significance (VUS) from consideration, the median VAF R² increased to 0.73 and 0.76 for TF and TMS respectively while other metrics were approximately unchanged.

TABLE 7
Calculated metrics from cohort.

| Median R² | Max(VAF) | Sum(VAF) | Mean(VAF | Median(VAF) |
|---|---|---|---|---|
| Tumor | 0.81 | 0.82 | 0.78 | 0.45 |
| Fraction | | | | |
| Tumor | 0.86 | 0.86 | 0.84 | 0.48 |
| Methylation | | | | |
| Score | | | | |

Table 8 shows demographics for the individual variant cohort that did not require multiple time points of data per patient:

TABLE 8
Demographics of individual variant cohort.

| Characteristic | N = 4,420¹ |
|---|---|
| Sex | |
| female | 2,271 |
| male | 2,094 |
| Unknown | 55 |
| Age | 64 (13) |
| Primary Tumor Organ | |
| Breast | 746 |
| Lung | 675 |
| CRC/Anal | 614 |
| Prostate | 344 |
| Pancreas | 319 |
| Skin | 283 |
| Other | 277 |
| Brain | 253 |
| Stomach/Esophagus | 235 |
| Liver | 142 |
| Ovary | 133 |
| Bladder | 122 |
| Kidney | 100 |
| Uterus/Cervix | 91 |
| Neuroendocrine | 61 |
| Head/Neck | 25 |
| Number timepoints, Mean (SD) | 1 (1) |

¹n; Mean (SD)

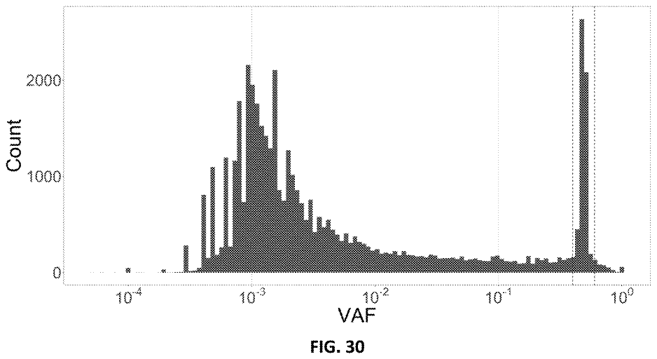

FIG. 30 shows the distribution of all detected VAFS, n>45,000 detections. All plots removed any VAF with 0.4<VAF<0.6 due to the presence of likely germline detections. The above plot demonstrates the cutoff (dashed line) corresponding to likely germline alleles in the plasma, shed by non-tumor cells.

The results demonstrated that VAF is highly correlated with TF and TMS.

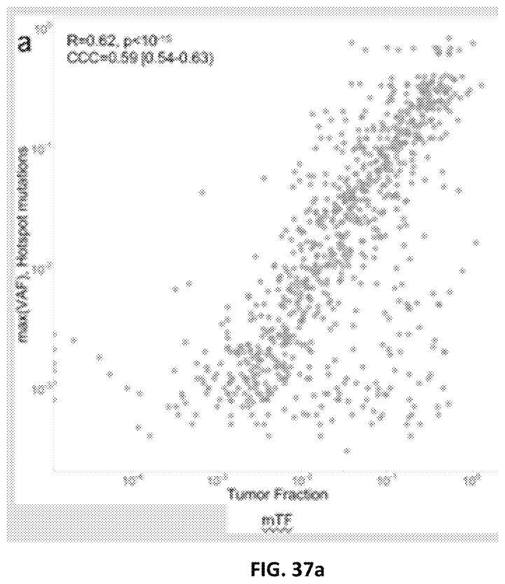

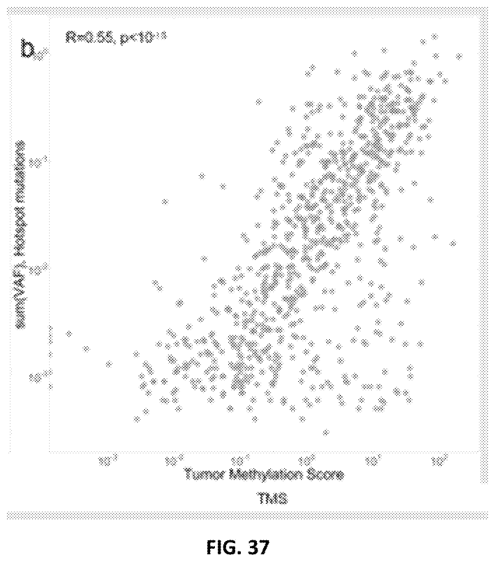

Table 9 shows the demographics for a cohort used to demonstrate correlation between mTF, TMS, max(VAF), and sum(VAF), the data for which is shown in FIG. 37. Subjects required one or more driver mutations. Only one longitudinal sample was included per each patient to eliminate bias towards patients with more samples. The distribution of tumor types is consistent with known incidence rates.

TABLE 9
Demographics of a cohort used to demonstrate correlation
between mTF, TMS, max(VAF), and sum(VAF).

| Characteristic | N = 852¹ |
|---|---|
| Sex | |
| Female | 470 |
| Male | 382 |
| Age | 63 (13) |
| Primary Tumor Organ | |
| CRC/Anal | 222 |
| Breast | 178 |
| Other | 145 |
| Lung | 101 |
| Pancreas | 87 |
| Stomach/Esophagus | 46 |
| Prostate | 35 |
| Ovary | 14 |
| Skin | 13 |
| Brain | 7 |
| Kidney | 4 |
| Number of Timepoints | 4.03 (1.61) |

¹n

| Characteristic | N = 171¹ |
|---|---|
| Sex | |
| Female | 86 |
| Male | 85 |
| Age | 62 (12) |
| Primary Tumor Organ | |
| CRC/Anal | 56 |
| Breast | 9 |
| Pancreas | 22 |
| Other | 15 |
| Stomach/Esophagus | 15 |
| Lung | 9 |
| Prostate | 8 |
| Ovary | 4 |
| Skin | 2 |
| Kidney | 1 |
| Number of Timepoints | 2.70 (1.34) |

¹n; Mean (SD)

Table 10 shows demographics for a cohort used for fold-change comparison between NSR and NSS, the data for which is shown in FIG. 35). The mean number of timepoints is shown, with the fold-change calculation taking the ratio of measurements between sequential timepoints for each patient. Fewer subjects were available when compared to Table 9 due to the requirement of longitudinal profiling with both NSS and NSR.

TABLE 10
Demographics of a cohort used for fold-
change comparison between NSR and NSS.

| Characteristic | N = 171¹ |
|---|---|
| Sex | |
| female | 86 |
| male | 85 |
| Age | 62 (12) |
| Primary Tumor Organ | |
| CRC/Anal | 56 |
| Breast | 39 |
| Pancreas | 22 |
| Other | 15 |
| Stomach/Esophagus | 15 |
| Lung | 9 |
| Prostate | 8 |
| Ovary | 4 |
| Skin | 2 |
| Kidney | 1 |
| Number of Timepoints | 2.70 (1.34) |

¹n; Mean (SD)

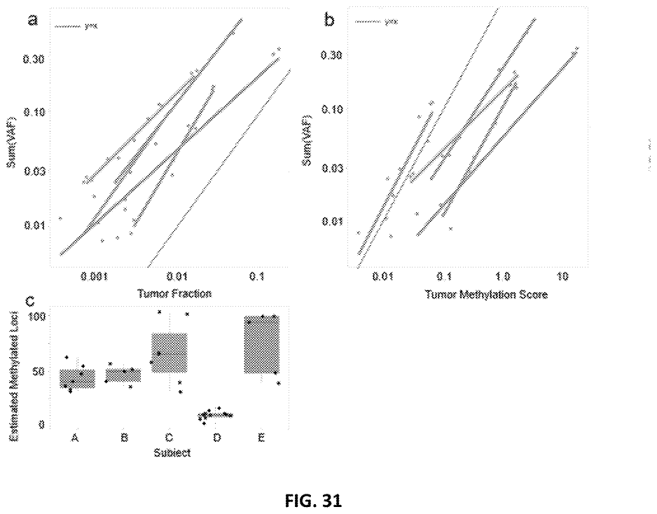

FIG. 31 illustrates that the longitudinal association between VAF-based TF estimates and methylome-based TF estimates is patient dependent. Five example subjects demonstrate variability in the association as evidenced by variable linear regression fits. Sum(VAF), as a measurement, depends on the number of detected mutations in the tumor. Similarly, whereas TF (a) simply estimates the proportion of cfDNA corresponding to ctDNA, TMS (b) includes methylation status. In addition to TMS and TF, NSR estimates the number of methylated loci (c). Subject D demonstrated fewer methylated loci (c) as evidenced by a relative shift in sum(VAF) association (b).

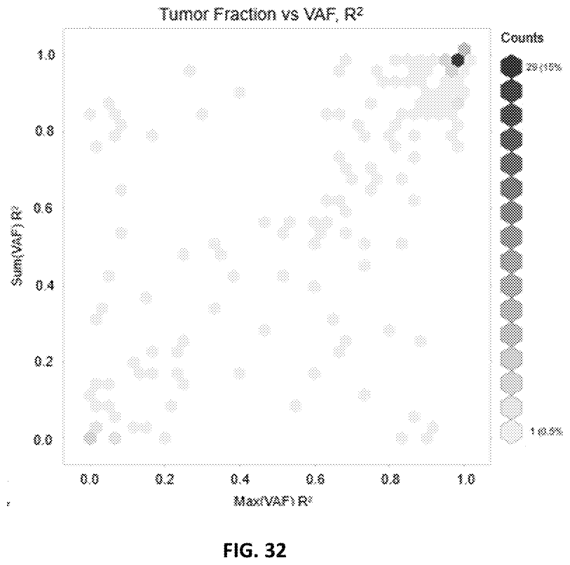

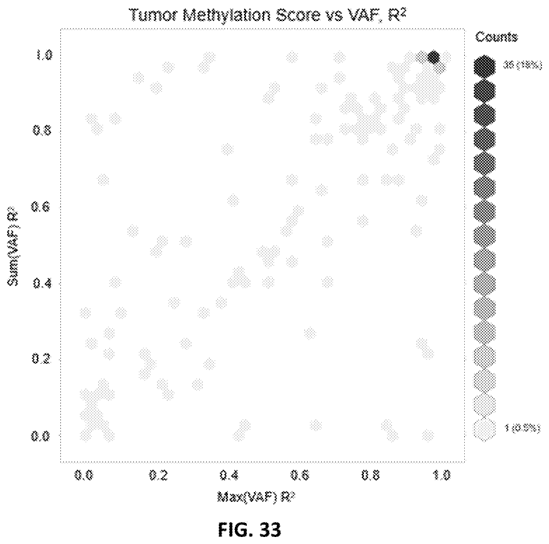

FIG. 32 is a 2D histogram of per-patient R² between TF and either sum(VAF) or max(VAF). Because correlations are patient-dependent, as shown in FIG. 31, the counts of each correlation range are rendered above. FIG. 33 is a 2D histogram of per-patient R² between TMS and either sum(VAF) or max(VAF).

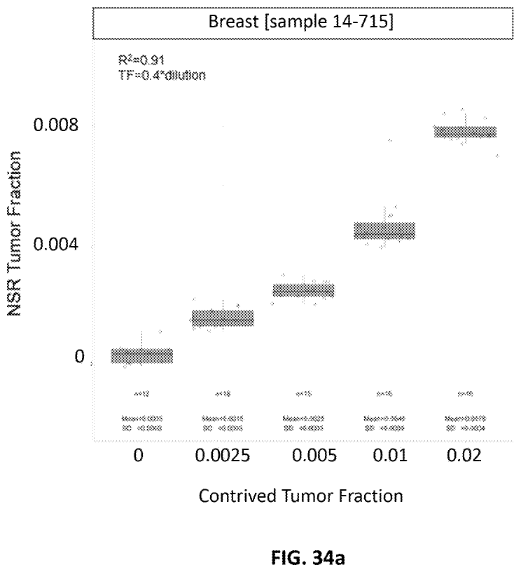

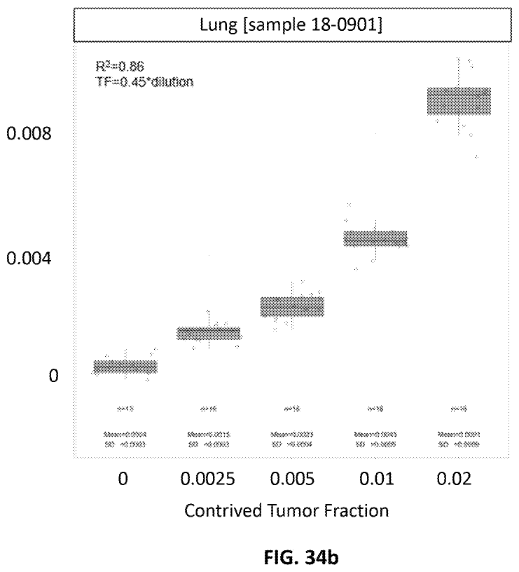

FIG. 34 illustrates that mTF is highly correlated with analytical tumor fraction samples. 16 independent replicate (buffy, tumor) pairs had their tumor fractions evaluated at four serial dilutions of a contrived tumor sample and a negative control. Pearson R² and Passing Bablok regression slopes are shown. Two tumor-derived DNA samples from breast (sample 14-715) and lung (18-0901) were serially diluted across four different in vitro concentrations and one negative control. Sheared tumor DNA was diluted with paired buffy sheared normal DNA, with the negative control consisting entirely of buffy-derived sheared DNA. 16 replicates were performed at each concentration excepting QC failures, with 16 paired replicates of buffy gDNA used as a background subtraction control for each replicate. Pearson's R² was used to compute correlation between the contrived TF and mTF, with 86% and 91% of variance in mTF explained by the TF concentration for lung and breast samples respectively, demonstrating high correlation in this analytical experiment.

FIG. 35 illustrates that serial fold-changes are significantly correlated between max(VAF) vs mTF (a) and sum(VAF) vs TMS (b). In a cohort of n=171 subjects with longitudinal profiling of both NSR and NSS, the first two landmark fold-changes from the first two on-treatment samples were included, or only the first in cases where only one on-treatment blood draw was taken, yielding n=231 measurements in total. Only driver mutations were considered, and only samples with measurements over 0.5% mTF/VAF were considered to reduce noise due to random sampling. Fold-change was computed as the ratio of measurement at any blood collection to the immediate prior blood collection. Typical fold-changes are approximately 10-fold, with outlier measurements on the order of 10²-fold. Both TMS and mTF were found to significantly correlate with sum(VAF) and max(VAF) respectively.

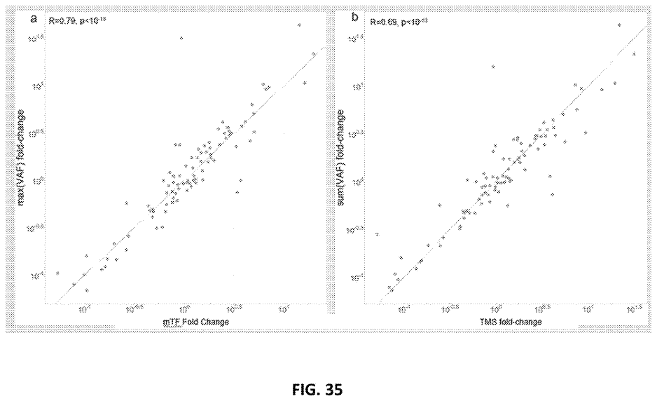

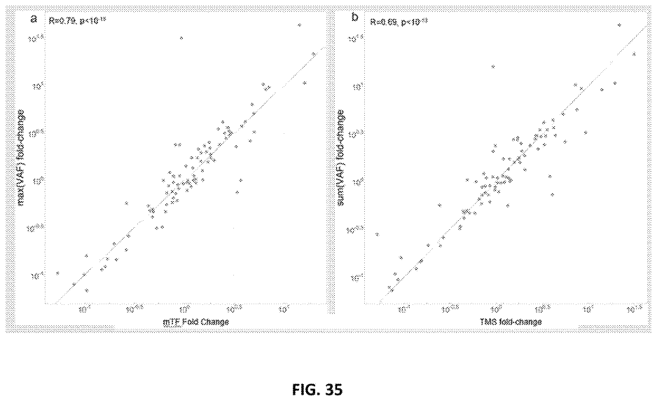

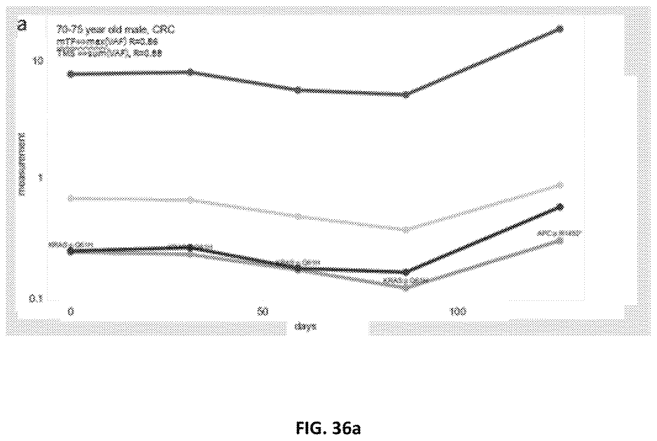

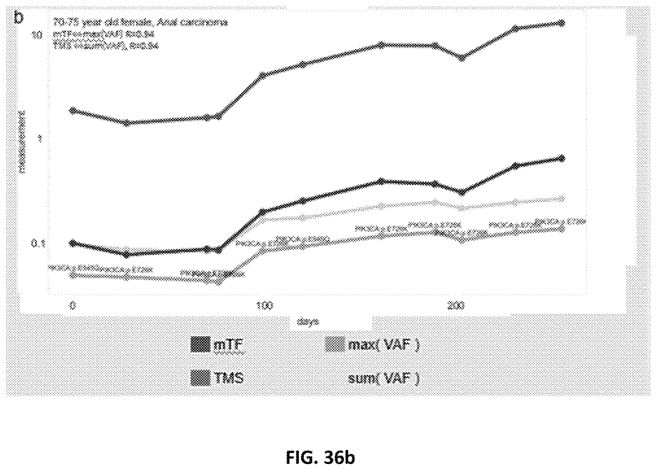

FIG. 36 shows individual cases comparing mTF, TMS, and sum/max of driver mutation VAFs demonstrate a high degree of clinical, longitudinal correlation. Max( ) and sumo summary statistics are shown across all driver VAFs, with the maximal-VAF gene and amino acid change labeled above each maximum. Baseline is taken as the first sample received for each subject. (a) represents a subject with a nadir measured at about 90 days and multiple driver mutations including KRAS Q61H and APC R1450*. (b) shows a more dense longitudinal collection in a subject with anal carcinoma with two PIK3CA mutations, E726K and E545Q, alternating as the potential max(VAF); two nadirs are visible during the treatment course, potentially due to therapeutic intervention. Each of these cases demonstrate a high degree of correlation between sum(VAF) and TMS, or max(VAF) and TF in a clinical and longitudinal context.

FIG. 37 illustrate that mTF correlates with max(VAF) (a) and TMS correlates with sum(VAF) (b). Driver mutations only were considered to avoid interference with variants of non-tumor origin such as germline or CHIP mutations. Correlation, though significant, is slightly reduced by clusters in the lower right corner which may represent CHIP events, subclonal tumor populations, or non-diploid copy number. The points on the diagonal are consistent with truncal mutations and highly correlated between measurements.

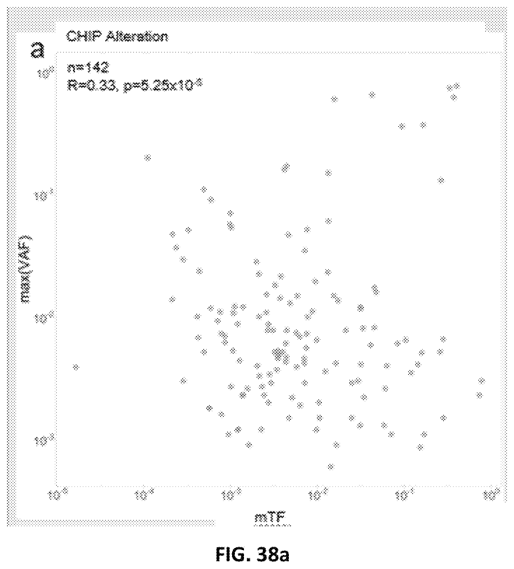

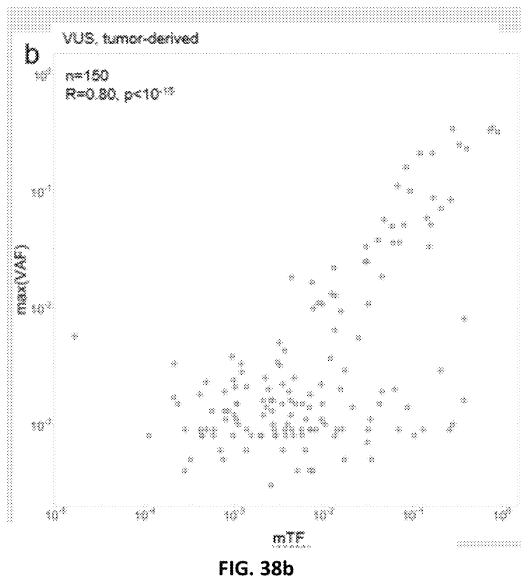

FIG. 38 illustrates that confirmed CHIP-positive mutations do not correlate with mTF and confirmed CHIP-negative VUs do correlate with mTF. Northstar Select distinguishes CHIP variants via gDNA sequencing. Max(VAF) cases with confirmed CHIP status are shown (a) compared to mTF. To expand our analysis outside driver mutations, we considered VUS mutations that were confirmed to be CHIP-negative (b), demonstrating high correlation in confirmed CHIP-negative samples. These results suggest VAF may be prone to CHIP interference when CHIP status is unknown, whereas mTF and TMS are resilient to the presence of CHIP mutations.

FIG. 39 illustrates CRC has more sub-VAF mutations. Each driver mutation in a sample was labeled as either maximum (i.e. VAF=max(VAF)), or in the case of multiple mutations, below max(VAF). Fisher's exact test identified CRC/Anal as containing a greater proportion of below-max VAF mutations versus the corpus of VAF rates in other tumor types. These data are suggestive of subclonal populations occurring more commonly in CRC tumors.

FIG. 40 illustrates that lower KRAS VAF:mTF ratio was observed only in CRC, and only for G12C and Q61H. KRAS mutation type and samples with multiple KRAS mutations are indicated. G12C is a targetable mutation in lung cancer; Q61H is seen in pancreatic cancer. Subclone-consistent clusters are enriched, only for G12C and Q61H, and only in CRC, suggesting interaction between specific KRAS variants, ctDNA shedding, and tumor type, and highlighting the potential of mTF to elucidate possible subclonal status.

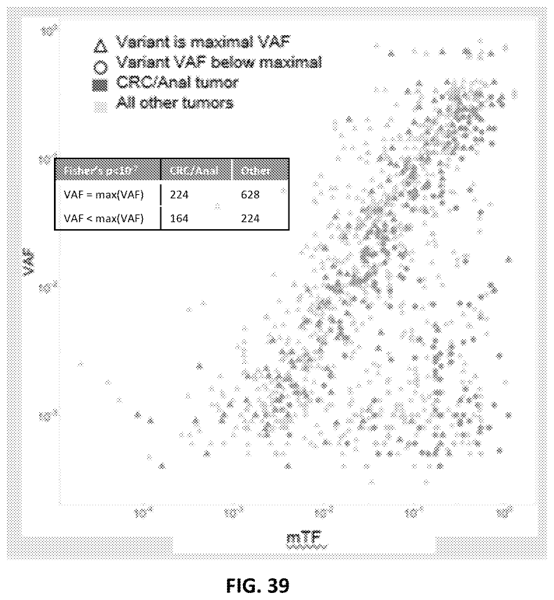

FIG. 41 illustrates the performance of TF estimates across tumor types. 11 broad classifications of tumor type are shown. Measurements, shown on a per-sample basis and not per-patient basis, align with known ctDNA shedding patterns e.g. CNS and pancreatic tumors are generally low shedders; CRC tumors are generally high shedders.

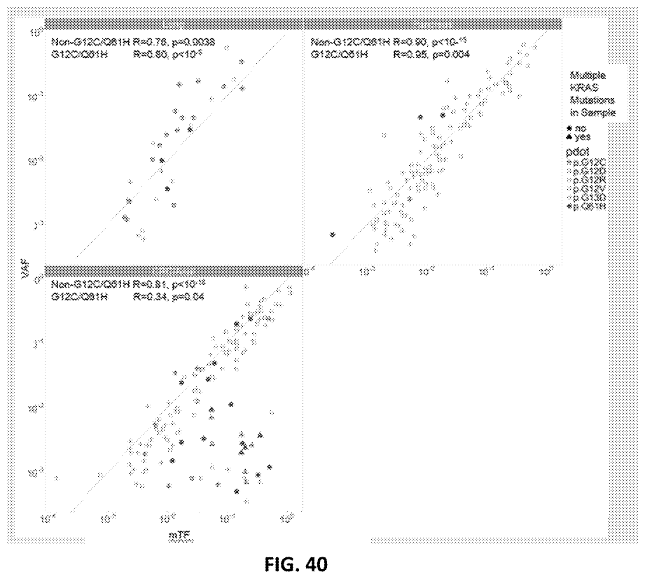

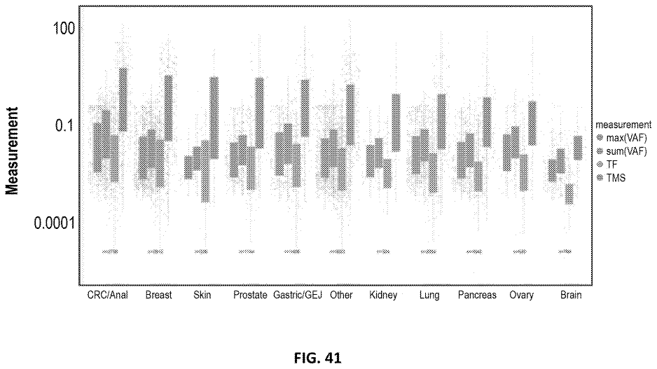

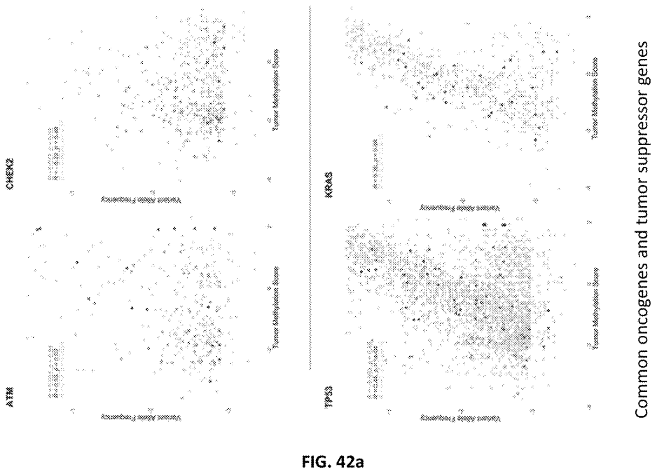

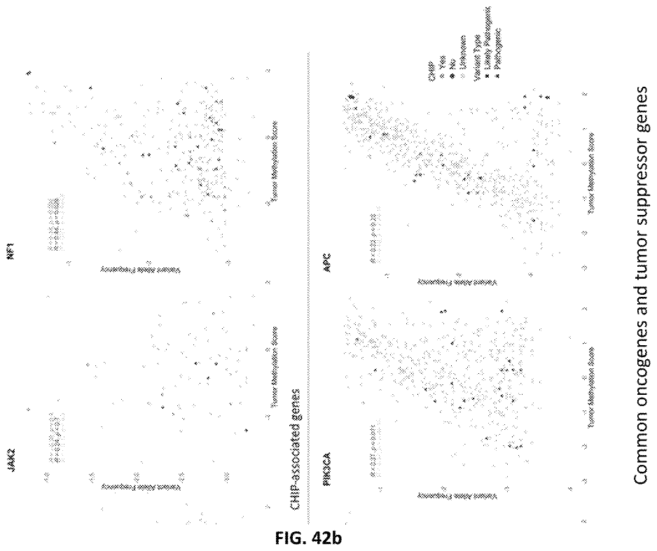

FIG. 42 illustrates that CHIP genes do not correlate with TMS. Northstar Select can distinguish CHIP variants with gDNA sequencing; CHIP status is overlayed where available. Four prevalent CHIP-associated genes are shown (top) above four prevalent cancer associated genes (bottom). In the case of TP53, positive CHIP detection events were observed off-diagonal versus on-diagonal for CHIP-negative somatic mutations. Variants of Uncertain Significance (VUS) are not shown.

### CONCLUSIONS

This example illustrates in a pan-cancer cohort of 852 patients, TMS and mTF were highly correlated with sum(VAF) and max(VAF) for driver gene mutations, with minimal correlation observed with CHIP. When considering CHIP-negative VUS mutations, a high correlation was also present. These findings together suggest that mTF possesses resilience against CHIP-originating non-ctDNA variants and highly correlates with only ctDNA-derived VAFs. Similarly, in a pan-cancer cohort of 196 patients, TMS and TF were highly correlated with VAF. Among all comparisons, TMS most highly correlated with sum(VAF). TMS does not correlate with orthogonally tested, CHIP-positive variants, both within and outside of CHIP-prone genes. This shows that Northstar Response is resilient to CHIP. Thus, TMS may serve as an alternate measurement to VAF, independent of available somatic mutations to query and resilient to non-tumor CHIP mutations.

Methylation-based TF produces estimates highly correlated with both in-vitro contrived samples and clinical VAF-based TF estimates such as max(VAF), realizing a more robust methylation-based alternative to max(VAF). Similarly, TMS may serve as a more robust alternative to sum(VAF). Correlations with VAF summary statistics such as max(VAF) or sum(VAF) may benefit from analysis on a per-patient vs aggregate level as these a prone to variability of available somatic mutations in any tumor.

A ZTP based tumor fraction TF estimation method produces estimates highly correlated with both in-vitro contrived samples and VAF-based TF estimates such as max( ) and sumo of VAF. These data show that TMS and mTF may serve as promising alternatives to sum(VAF) and max(VAF) in research and clinical settings, with evidence of a capacity to overcome known limitations of VAF-based approaches such as CHIP.

Colorectal cancer cases were found to have a greater frequency of lower-VAF driver mutations, corroborated by the observation of a mutant allele specific reduction in VAF, seen only for specific KRAS variants and only in colorectal cancer. These data highlight the potential of mTF to uncover complex associations between tumor genomics, ctDNA, and tumor biology.

### Example 13: Comparison of Different Tumor Fraction Determination Methods

A “fully methylated” DNA sample was created by in vitro methylation of human genomic DNA to represent a tumor sample. Since >90% of non-tumor cfDNA originates from blood cells, the in vitro methylated DNA was diluted in WBC DNA to recapitulate both tumor and non-tumor specific DNA methylation patterns.

Contrived samples were generated with tumor fraction (TF) ranging from 0.01% to 10% and total input DNA ranging from 30 ng to 100 ng and tested using the 2214-plex ctDNA assay with absolute quantification at each locus.

Multiple Tumor Fraction Analyses were Performed on the Data:

Average TF: The methylation fraction at each locus with detected methylated DNA was computed, and then the methylation fraction measurements were averaged across all detected loci. Loci that exhibited methylation in the WBC were excluded. Since 30 ng corresponded to ~1000 GE at each locus, a detected locus could have a minimum methylation fraction of only 1/1000. This lower bound was recapitulated in the inaccuracy of Avg TF measurements at TFs<0.1%, which plateaued at the expected 0.1%.

Zero-truncated Poisson (ZTP): The histogram of detected DNA molecules was fit to a zero-truncated Poisson distribution. The number of loci with 1 molecule, 2 molecules, etc. was computed. Assuming that the methylated molecules at each locus is identical Poisson distributed, the average methylation across all loci was estimated using the truncated Poisson distribution. The ZTP estimator achieved better results than the AVG TF method.

ZTP with WBC loci exclusion: Same analysis as above, but loci that exhibited methylation in the WBC were excluded. This improved accuracy compared to analyzing all loci.

ZTP with background subtraction: ZTP analysis with additional background subtraction applied based on aggregate measurements of DNA methylation in WBC. This approach provided the best accuracy, especially at low tumor fractions.

### Example 14: Tumor Fraction Measurements Across Different Methylation Loci

Forty-one tumor biopsy samples were obtained across a diverse set of cancer primaries (e.g., colorectal, breast, and non-small cell lung cancer). DNA was extracted from tissue samples, underwent bisulfite conversion, and was subjected to the 2214-plex methylation assay.

The ZTP tumor fraction was calculated on separate sets of 816 and 517 loci (“A” and “B” respectively). The assessed tumor purity of these samples ranged from <10% to 40%.

The tumor fraction calculated from these loci subsets demonstrated high reproducibility (R²=96%), demonstrating the ZTP method's ability to robustly calculate tumor fraction even when the measurements are obtained on different loci.

### Example 15: Correlation with Variant Allele Fraction

Blood was collected from cancer patients and analyzed for somatic mutations in KRAS and TP53. The blood was also processed through a >500-plex assay for DMRs specific to tumors.

The methylated tumor fraction (TF) was measured at each locus and compared with the KRAS and TP53 variants. KRAS VAFs correlated very well with methylated TF, with R²=0.84.

The TP53 VAF and methylation TFs showed a cluster of samples that correlated well (R²=0.76) and a distinct cluster of samples that had TP53 VAF that was >10-fold lower than the expected methylation TF. These alterations could represent a minority population of tumor cells harboring a TP53 variant.

### Example 16: Methods for Tumor-Naive Tumor Fraction Measurements at Sub-Molecule Limit of Detection

Quantification of circulating tumor DNA is increasingly used to monitor disease progression in multiple clinical settings. In early-stage cancers, detection and quantification of ctDNA is used for adjuvant and surveillance. In late-stage cancer, ctDNA can be used to assess treatment response and monitor for cancer progression.

There is a need for ctDNA assays that do not rely on tumor tissue. Tumor biopsies are obtained infrequently, typically only at the point of first diagnosis. Over time, the initial tumor biopsy may no longer be representative of a heterogeneous cancer that (1) partially responds to therapy, (2) acquires therapy-resistant cancer subclones, (3) metastasizes to distal anatomical sites. Furthermore, a tumor biopsy is inherently a localized sample of cancer cells, and it may not be reflective of other cancerous lesions.

So-called bespoke ctDNA assays have been created that first determine a set of tumor-specific genomic variants through analyzing a tumor tissue sample. A custom ctDNA assay is then created for each patient based on the alterations found in the tissue. However, the bespoke approach suffers from logistical challenges associated with acquiring and analyzing tissue, which can increase assay failure rates and delay results. Furthermore, such assays may not capture details of cancer evolution, because they are designed against the initial tumor sample. Therefore, bespoke assays may miss cancer progression events associated with therapy resistance and failure, metastasis and spread to distant sites, and acquisition of new genetic features.

Determination ctDNA fraction has several other useful applications. The variant allele fraction of somatic alterations detected in ctDNA can be compared with the overall ctDNA fraction to assess whether the somatic variant is consistently present in all tumor cells or if the somatic variant is only present in a subclonal population. This information can be valuable for detecting the emergence of resistant subpopulation indicated by a resistance mutation (e.g. EGFR T790M is resistant to EGFR tyrosine kinase inhibitor), or in treatment decisions such as applying a therapy targeted against particular alterations. The ctDNA tumor fraction can also be used to assess absence of a mutation, which is useful in certain clinical contexts. For example, KRAS wild-type colorectal cancer is treated differently than KRAS mutated cancer.

An accurate determination of KRAS wild-type status is only possible when the ctDNA methylation fraction exceeds the limit of detection threshold for KRAS mutations. Despite the advantages of measuring ctDNA tumor fraction independently of a tumor tissue sample, there are several inherent challenges to overcome. There may be non-cancer specific signals in cell-free DNA. Examples include clonal hematopoiesis and epigenetic drift, which are somatic genomic and epigenomic alterations, respectively.

This example presents a new method for accurately measuring ctDNA tumor fraction. Absolute quantification of hundreds of tumor-specific differentially methylated regions is performed in multiplex on a cell-free DNA sample from blood. Statistical methods operating on count data such as zero truncated poisson are applied to accurately determine the tumor fraction even when there is <1 tumor DNA molecule per locus on average. Such scenarios are common, such as in a typical blood sample containing only 1000 haploid genomic equivalents (GE) of cfDNA and a tumor fraction <0.1%.

### Methods

First, the blood sample is isolated into plasma and buffy coat, and DNA extraction is performed on each portion to obtain cfDNA and WBC DNA, respectively. Both DNA specimens are subjected to bisulfite conversion so that CpG methylation sites are unchanged and nonmethylated cytosines are converted to uracils. A multiplex PCR assay is then performed to specifically amplify >2000 DMR loci. The assay also includes Quantitative Counting Templates, synthetic DNA with a embedded molecular identifier and PCR primer binding regions that co-amplify with the sample DNA. After PCR amplification, the sample is indexed and NGS is performed. The NGS data generated is then analyzed. The OCT-derived sequencing reads are used to determine the number of sequencing reads per DNA molecule at each locus, which is then used to determine the absolute quantification of sample-derived DNA. This method results in absolute quantification of methylated DNA at each of the >2000 loci.

Digital PCR is a widely used method for absolute quantification of DNA. However, it is limited to multiplexing of only 4-6 loci. By obtaining absolute quantification measurements across hundreds of loci, statistical models of counting noise can be successfully applied to determine the average number of molecules per locus. For example, sampling noise of rare molecules is theoretically described by the poisson distribution. However, when the mean counts is near or below 1, there is significant probability of obtaining 0 counts.

In a tissue-naive setting, it is not known a priori which of the 2000 loci are actually methylated in the tumor. Detecting a methylated DNA molecule at a locus provides evidence that the tumor is methylated at that locus. Yet, not detecting a methylated molecule could be either due to lack of methylation in that particular patient's tumor or sampling noise. At high tumor burdens (e.g. >0.1% tumor fraction), the probability of obtaining O molecules in a locus is low, sampling noise is negligible, and the tumor fraction can be accurately obtained by averaging tumor fraction measurements across the detected loci. However, at low tumor burden, averaging across only detected loci would result in a severe over-estimate of TF. Alternatively averaging across all loci (both detected and non-detected) would result in a severe under-estimate because many of the loci may not actually be methylated in the tumor. The utility of highly multiplexed absolute quantification is that truncated or zero-inflated models can be used that do not require a priori knowledge of precisely which loci are methylated in the tumor. Rather, the proportion of loci at different non-zero levels (e.g. 1 molecule, 2 molecules, 3 molecules, etc.) is used to fit the average number of methylated DNA molecules across all loci.

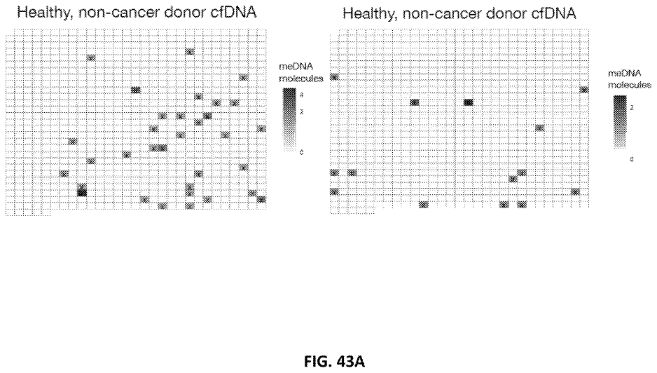

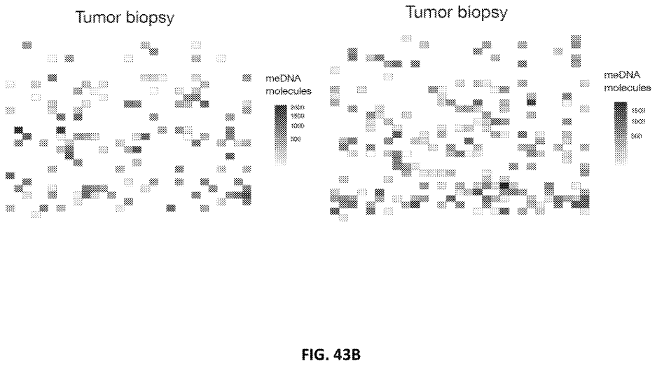

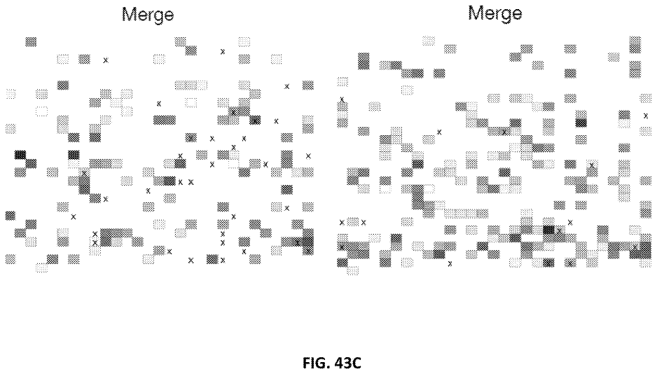

FIGS. 43A-43C show representative examples of methylated DNA in healthy blood and tumors. Since a tumor sheds its genetic material into the bloodstream as circulating tumor DNA, the overall cell-free DNA is a mixture of DNA originating from both tumor and non-tumor cells. A subset of 816 loci with similar molecule capture and amplification efficiency characteristics was identified. Representative examples are shown for two healthy blood samples (FIG. 43A) and two tumor biopsy tissues (FIG. 43B). Each tile represents a locus, and the number of detected methylated DNA molecules is indicated by the tile shading. Healthy cfDNA and tumor DNA are overlayed in (FIG. 43C), with the loci detected in the healthy sample indicated by an ‘x’. Healthy cfDNA exhibited far less DNA methylation than tumor tissues. Furthermore, the presence or absence of DNA methylation for a given locus is highly variable across healthy cfDNA and tumor specimens. Thus, methods for calculating tumor fraction from cfDNA need to be robust to the patient-dependent tumor methylation that is unique to that individual's tumor. Furthermore, the method should be able to exclude loci that exhibit methylation in non-tumor sources (e.g. white blood cells).

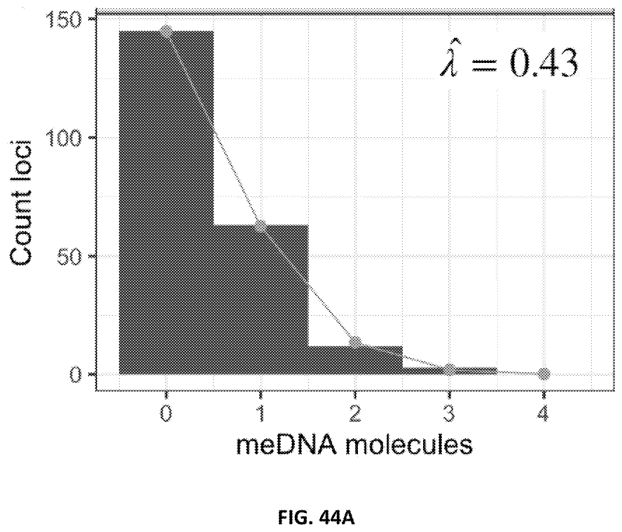

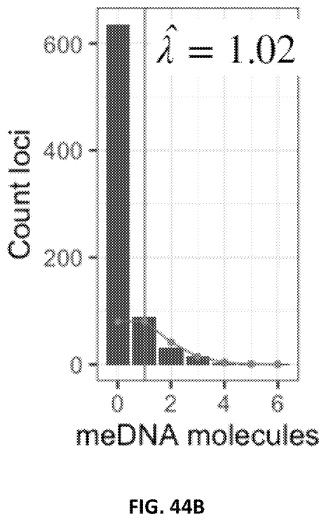

FIGS. 44A and 44B show that absolute quantification across hundreds of loci enable tumor-naive statistical reconstruction of tumor fraction at sub-molecular limit of detection. FIG. 44A shows a representative example of a 0.01% TF contrived sample (in-vitro methylated DNA diluted to 0.01% in a background of healthy WBC DNA). The frequency of loci with 1, 2, 3, etc. molecules in this sample was used to fit a zero truncated poisson model, which resulted an estimate of 0.43 molecules per locus. Since the assay measured 4370 GE of input DNA, this represents a TF measurement of 0.0098%, in good agreement with the contrived dilution of 0.01%. Crucially counting models such as zero-truncated poisson is able to use the relative frequency of loci with 1, 2, 3, etc. counts to obtain an accurate estimate even in scenarios when there is on average less than 1 molecule per locus. FIG. 44B shows an example of a tumor biopsy tissue diluted 200× into buffy coat DNA background. The ZTP fit of 1.02 molecules per locus is shown. The inflated numbers of loci with 0 methylated DNA (meDNA) is expected because a given tumor will only exhibit hypermethylation in a fraction of the loci in the panel. The strength of the zero-truncated poisson fit that precise knowledge of which loci in the tumor are methylated is not needed, thereby enabling tumor-naive tumor fraction measurements.

To evaluate the specificity of the DMR loci for tumor DNA, the multiplex assay was performed on age-matched healthy individuals (n=54). The methylation panel was separately performed on both cfDNA and white blood cell (WBC) isolated from each blood sample. The average amount of cfDNA analyzed was 1160 haploid genomic equivalents (GE) [min 780 GE, max 1800 GE]. The average gDNA analyzed was 7400 [min 5000, max 14300). 2214 differentially methylated loci were analyzed in multiplex for each sample.

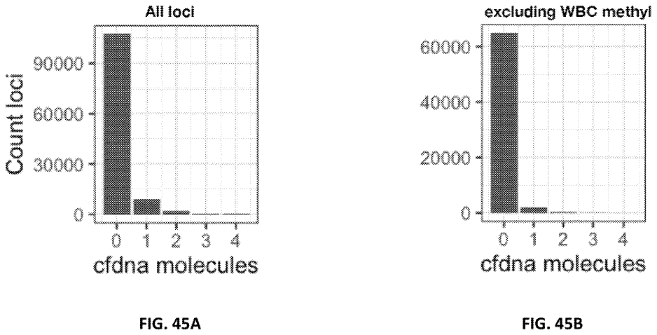

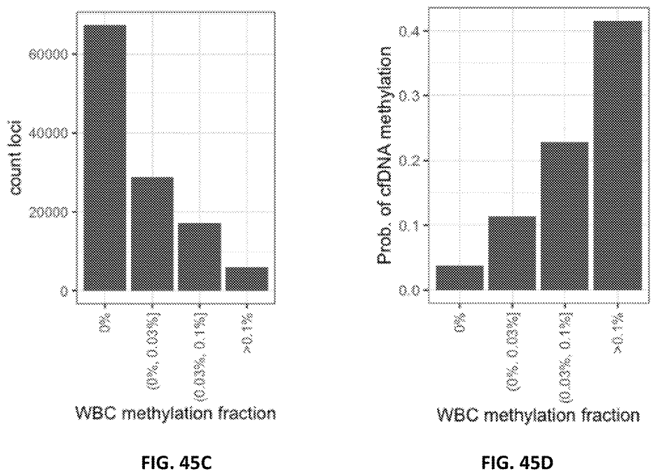

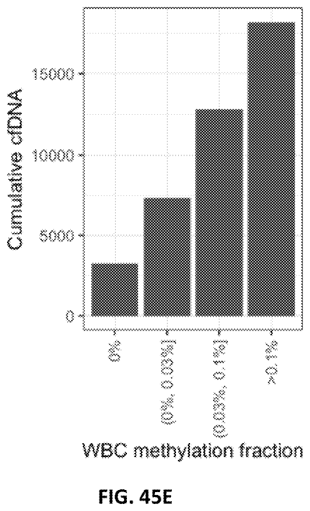

There were 119,556 WBC measurements and 119,556 cfDNA measurements across all patients and loci in the panel. Across all cfDNA measurements, 90% had no detectable methylation (FIG. 45A). It is known that most cfDNA originates from blood cells. Therefore, DNA methylation found in cfDNA should also be present in the corresponding WBC measurement. When cfDNA measurements are excluded on the basis of detected methylation in the matched WBC locus and sample, 96% of cfDNA measurements had no detectable methylation (FIG. 45B).

Further analyses were performed to assess the ability of WBC to predict non-tumor methylation in cfDNA. Over half of the WBC measurements had no detectable DNA methylation (FIG. 45C). When methylation was detected in WBC, the proportion of methylated DNA molecules at that locus (methylation fraction) was low, with most measurements showing a methylation allele fraction of <0.1%. Furthermore, the frequency of detecting methylation in cfDNA was correlated with the WBC methylation fraction (FIG. 45D). At relatively high methylation fractions of 0.1% or higher in WBC, methylation was also detected in cfDNA over 40% of the time. Lower rates of cfDNA methylation detection could be due to poisson sampling effects. At a methylation rate of 0.1% and input cfDNA mass of 1 000 GE, there would only be 1 methylated molecule on average. Based on Poisson counting noise, there would be no detection in 37% of the measurements. Thus, the decrease in cfDNA detection frequency at lower WBC methylation fractions is at least partially explained by counting noise. The cumulative sum of methylated cfDNA molecules was assessed as WBC methylation fraction increased (FIG. 45C). 80% of the methylated cfDNA was found in loci that also had corresponding methylation in WBC. Overall, the impact of this finding is that specificity of cfDNA assays can be improved by analyzing the buffy coat and excluding the loci found to be methylated in WBC from the analysis of cfDNA.

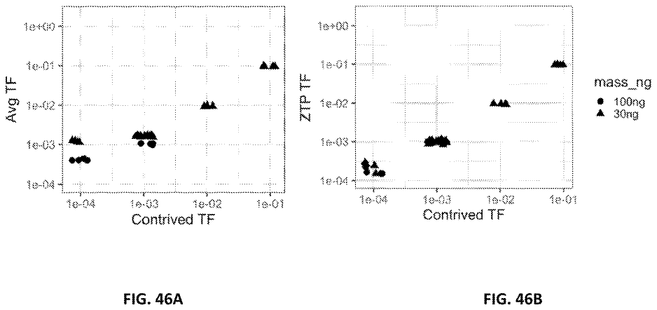

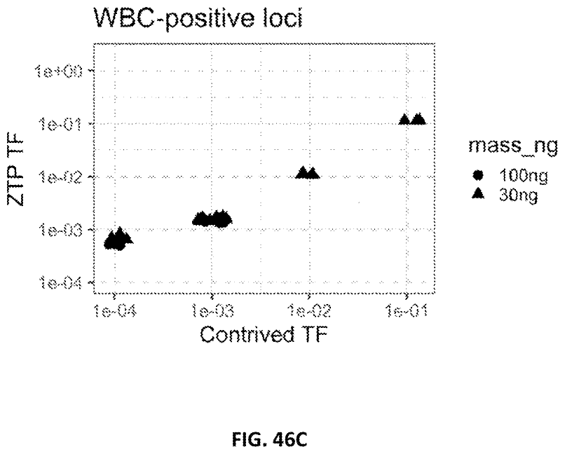

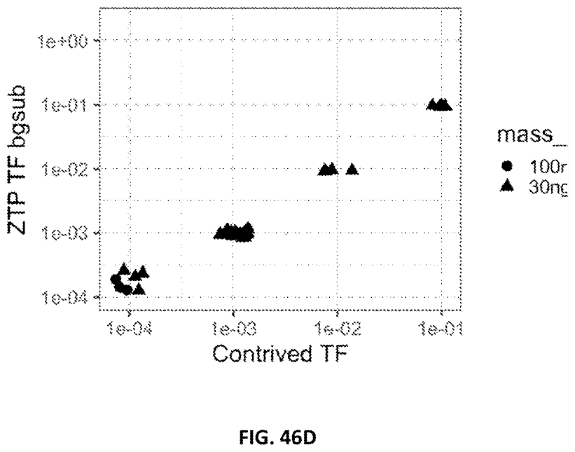

Since >90% of non-tumor cfDNA originates from blood cells, the in vitro methylated DNA was diluted in WBC DNA to recapitulate both tumor and non-tumor specific DNA methylation patterns. Contrived samples were generated with tumor fraction (TF) ranging from 0.01% to 10% and total input DNA ranging from 30 ng to 100 ng and tested using the 2214-plex ctDNA assay with absolute quantification at each locus. Multiple tumor fraction analyses were performed on the data. FIG. 46A shows the average TF analysis computed by determining the methylation fraction at each locus with detected methylation DNA, and then averaging the methylation fraction measurements across all detected loci. Loci that exhibited methylation in the WBC were excluded. Since the 30 ng corresponded to ~1000 GE at each locus, a detected locus could have a minimum methylation fraction of only 1/1000. This lower bound is recapitulated in the inaccuracy of Avg TF measurements at TFs<0.1%, which plateaued at the expected 0.1%. FIG. 46B shows how the zero-truncated poisson (ZTP) method fits the histogram of detected DNA molecules to a zero-truncated poisson distribution. Briefly, the number of loci with 1 molecules, 2 molecules, etc. is computed. Assuming that the methylated molecules at each locus is identical poisson distributed, the average methylation across all loci may be estimated using the truncated poisson distribution. Crucially the zero-truncated poisson distribution does not incorporate loci that had no detected molecules. Thus, the estimate is robust against variable numbers of loci that are truly methylated for a given tumor. The ZTP estimator achieves better results than the AVG TF method. Loci that exhibited methylation in the WBC were excluded. FIG. 46C shows the same analysis as FIG. 46B, but across all loci, including those that had methylation in WBC. Measurements were over-estimated at lower contrived TFs, demonstrating that loci exclusion based on WBC improves accuracy.

FIG. 46D shows a ZTP analysis with additional background subtraction applied based on aggregate measurements of DNA methylation in WBC. Excluding WBC-methylated loci from TF analyses improves specificity and accuracy. However, WBC methylation detection is also subject to poisson sampling effects and can loci that have very low levels of WBC methylation. A ZTP fit is also applied to WBC measurements to estimate the true rate

WBC methylation, even among loci did not detect any methylated DNA molecules (see FIG. 3). To obtain a “background subtracted” TF measurement, the ZTP TF measurement is then scaled in proportion to the expected amount of methylation from WBC.

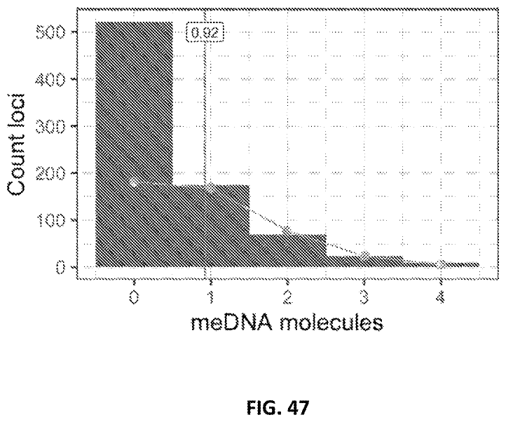

FIG. 47 shows a representative example of WBC methylation and zero-truncated poisson fit. 522 loci had 0 detected in WBC methylation from the blood of a healthy donor. ZTP fit of loci that detected low levels of DNA methylation implies that 167 out of the 522 loci are methylated at a rate of 0.92 molecules. The amount of WBC analyzed was 4710 GE, therefore these 167 loci exhibit a methylation fraction of 0.02%. Thus, these 167 loci are expected to contribute 0.033 methylated molecules per input GE in the cfDNA sample as non-tumor background. Tumor fraction measurements of the cfDNA can be “background subtracted” by applying a correction factor of (methyl_cfdna−methyl_bg)/methy_cfdna. methyl_cfdna is the sum of methylated cfDNA molecules per input GE. methyl_bg is the expected sum of non-tumor methylated molecules per input GE, which is 0.033 in the above example.

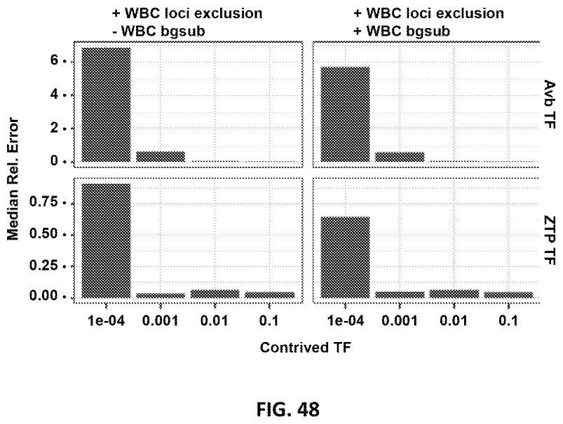

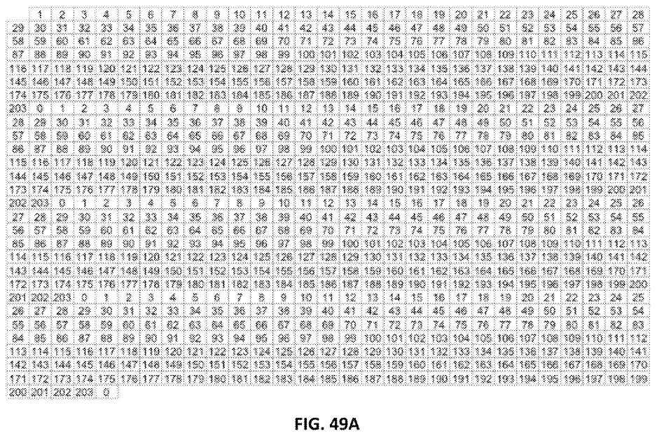

FIG. 48 shows a summary of accuracy of tumor fraction algorithms. The “average TF” approach overestimates the TF by a median factor of >6-fold at 0.01% tumor fraction. At 0.1%, the median relative error is >50%. Implementing WBC background subtraction (WBC bgsub) improves the accuracy, but the median error is still a >5-fold overestimate compared to the ground truth. ZTP methods have much better accuracy than average TF. The median relative error of ZTP with the WBC bgsub is 60% at the 0.01% TF level and <5% error at the 0.1% level.

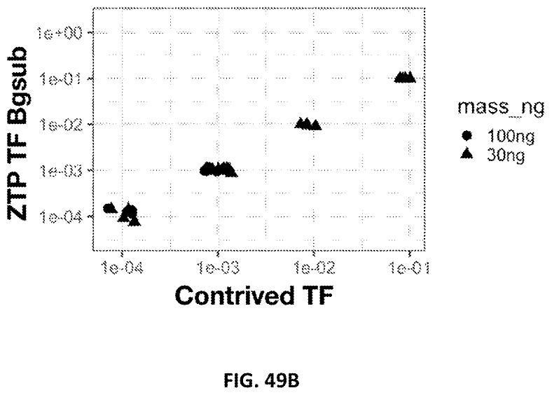

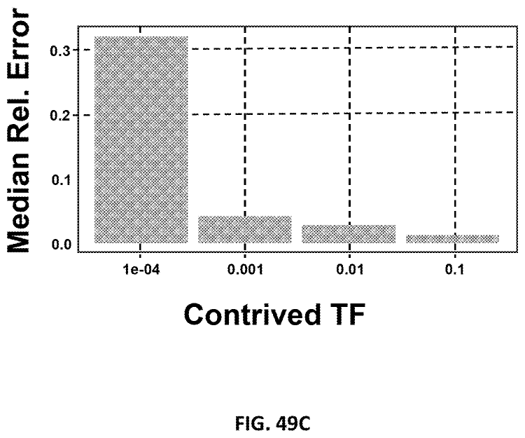

Loci blocks were assigned 4 loci each to represent a haplotype block (FIG. 49A). Tumor-specific DNA methylation should co-occur across all loci within each haplotype block. Within each block, the absolute counts of methylated DNA from the 4 loci were summed to obtain a count of the methylated DNA molecules for that block. The ZTP method was then performed as before, with the exception that the analysis was performed on loci blocks rather than individual loci. Grouping loci into blocks further improved the tumor fraction accuracy across contrived TF ranging from 0.01% to 10% (FIG. 49B). Previously, the best relative error obtained at 0.01% was 60%. Loci block analysis decreased the error by half, to ~30% relative error (FIG. 49C).

### Tumor Fraction Measurements are Highly Stable Across Different Methylation Loci.

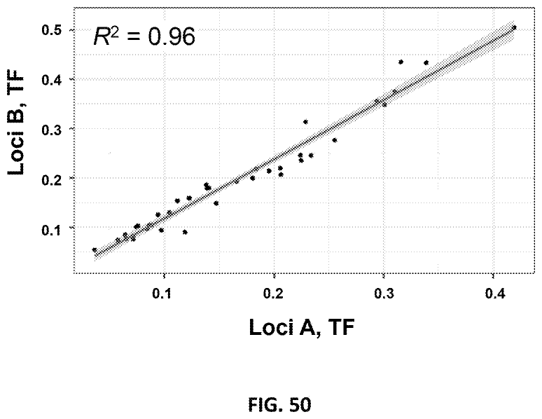

Forty-one tumor biopsy samples were obtained across a diverse set of cancer primaries (e.g. colorectal, breast, and non-small cell lung cancer). DNA was extracted from tissue samples, underwent bisulfite conversion, and subjected to the 2214-plex methylation assay. The ZTP tumor fraction was calculated on separate sets of 816 and 517 loci “A” and “B” respectively. Results are shown in FIG. 50. The assessed tumor purity of these samples ranged from <10% to 40%. The highly reproducible tumor fraction calculated from these loci subsets (RA2=96%) demonstrates the ZTP method's ability to robustly calculate tumor fraction even when the measurements are obtained on different loci.

Methylated Tumor Fraction Estimates Match Variant Allele Fraction (VAF) Obtained from Driver and Non-Driver Somatic Mutation

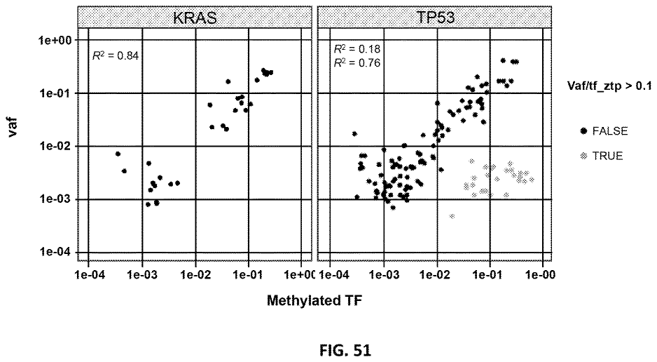

Blood was collected from cancer patients and analyzed for somatic mutations in KRAS and TP53. The blood was also processed through Northstar TRUE Response, a >500-plex assay for DMRs specific to tumors. The methylated tumor fraction (TF) was measured at each locus and compared with the KRAS and TP53 variants. Results are shown in FIG. 51. KRAS VAFs correlated very well with methylated TF, with RA2=0.84. The TP53 VAF and methylation TFs showed a cluster of samples that correlated well (RA2=0.76) and a distinct cluster of samples that had TP53 VAF that was >10-fold lower than the expected methylation TF. These alterations could represent a minority population of tumor cells harboring a TP53 variant.

### CONCLUSIONS

Tissue-naive assays are useful for several reasons. They are robust to evolution; convenient, have fast turnaround time, and work when tissue biopsy is infeasible. However, measuring tumor fraction is challenging because: (1) no a priori knowledge of tumor-specific genetic/epigenetic changes; (2) contribution of cfDNA from non-tumor specific sources, like CHIP or epigenetic drift; (3) very scarce amounts of tumor DNA means very low reproducibility on a per-locus level.

Highly multiplexed absolute quantification solves these challenges. Accurate tumor fraction measurements are obtained by (1) converting sequencing data to molecule counts and (2) statistical algorithms operating on molecule count data from hundreds of loci.

TF w. ZTP enables accurate measurements at sub-molecular levels (<1 ctDNA molecule per locus on average) without a priori knowledge of the alterations present in the tumor. Specificity and accuracy is improved by characterizing non-tumor specific noise coming from leukocytes and removing those loci from analysis. This is enabled by the tumor-naive approach (FIGS. 43A-43C).

The TF algorithm was run on cancer patient plasma and buffy coat DNA. Comparing KRAS variant allele fraction and methylation TF gave good results.

### Example 17

Northstar Response is an epigenetic assay of cfDNA intended for monitoring response to therapy and progression for advanced cancer patients. Over 500 epigenetic loci are interrogated that are differentially hypermethylated in cancer. The “Total Methylation Score” (TMS) is essentially the total tumor methylated molecules observed in cfDNA, normalized to the amount of input DNA into the assay. One limitation of the TMS is that it is a property of both the assay and the sample. A different assay that used different loci should return a different TMS even on technical replicates.

Instead, obtaining a “tumor fraction” estimate from the NSR assay has several advantages:
Tumor fraction is a property of the sample alone, so the assay can be improved independently of the reports. We can have a easier transition from the NSR v1 and NSR v2 assays.
Enables validation with orthogonal biomarkers and measurement techniques. We can compare tumor fraction against SNP VAF. We can compare methylation fraction with ddPCR.
Enables clinical correlation between Response and Select. SNPs can be assessed to be tumor-derived by comparison with tumor fraction.

Tumor fraction is obviously the fraction of tumor-derived DNA to total DNA molecules:

$$\frac{{N}^{t u m o r}}{{N}^{total}} .$$

In the case of a SNP, the allele fraction is straightforwardly the estimate of the tumor fraction. As an example, when a BRAF V600E mutation is observed at 2% VAF, then we conclude that the tumor fraction is 2%.

However, the calculation is more complicated when examining multiple SNPs at low VAF, i.e. only a few molecules per locus. TP53 is also commonly mutated in cancer. If a patient has a BRAF V600E at 0.2% VAF and a TP53 R273H at 0.3% VAF, we should average them together to estimate the tumor fraction as 0.25%. What if no TP53 mutations are observed? The 0.2% tumor fraction based on V600E alone would be an overestimate, because it was “lucky” that we happened to observe that 1 mutation. There are probably other SNPs that are also present in the tumor, but they were not observed due to sampling noise.

For a tumor-free assay looking at a panel of (epi)genetic loci, a key challenge in estimating the tumor fraction is therefore the uncertainty in whether that locus is actually altered in the sample's tumor DNA. The Nₜₒₜₐₗ is an unknown parameter that needs to be fit from the data.

This example shows that tumor fraction can be estimated by modeling the methylated QE from each locus as independent and identical poisson distributions. The observed histogram of molecules per locus is therefore generated by a poisson distribution in which λ corresponds to the tumor fraction. For any given locus, we do not know a priori whether it is methylated in the tumor. Therefore, a conditional poisson distribution is used to fit \hat{λ}:P(k|k>0), where k is the number of molecules (also known as a “zero truncated poisson” distribution). This allows us to ignore the loci that contribute O methylated molecules in the fit.

Northstar Response v1 LOB and LOO data were reanalyzed to show that the tumor fraction estimator has low background on healthy cfDNA and has good accuracy/linearity in the range of 0.25%-2% tumor fraction. ~100 cancer cfDNA specimens were also analyzed and show that TF correlates with TMS and VAF. TF and TMS correlate with R2=0.88 (log-scaled). Finally, it was shown that methylation TF estimates are correlated with a distinct subset of somatic SNPs. Furthermore, methylation TF is uncorrelated with a separate subset of SNPs with low VAF, suggestive of CHIP. Thus, a SNP with VAF inconsistent with methylation TF could potentially be used to identify CHIP alterations.

### Theory

### Estimating Δ

It is assumed that each locus is identically distributed and is poisson with mean number of methylated molecules λ. Additionally, only a subset of the low-background loci are methylated in the tumor. This is unknown for each patient.

Recall that P(k)=λ^{k}e^{-λ}/k! Here are 4 ways of estimating X:

When λ<<1, λ≈P(k=0).

So it merely needs to be determine what proportion of loci that exhibit k>0. The drawback of this approach is that it is not known how many loci are methylated (N)! The other methods allow for the estimation of both λ and the number of methylated loci.

Reminder: P(k=0)=e^{-λ}≈1−Δ.

When λ≈1 simply take the ratio of observations.

$$P ( k ) / P ( k - 1 ) = \frac{λ}{k} .$$

With λ in hand, P(k=0) can be calculated to recover N. This method works best when λ≈1 because the data will be clustered around k=1, 2, 3. The increased counts results in a better estimate. In practice, taking the ratio of P(k=2)/P(k=1) works best because it avoids the k=0 case.

When lambda >>1, the mean and variance can be taken, both of which provide independent estimates of λ. With λ>>1, the k=0 datapoint can safely be dropped without affecting the estimate by much. Use the “zero truncated poisson” moments estimate:

$$\overline{k} = \frac{\hat{λ}}{1 - {e}^{- \hat{λ}}} for k > 0 .$$

This method works the best in both theory, simulation, and clinical data

### Limit of Blank

Limit of blank data was Northstar Response v1 run on cfDNA and gDNA obtained from healthy volunteers (n=138). An additional 16 samples were included from pregnant people, as a positive control.

LOB cfDNA

The calculations are as follows:
1. Measure buffy coat. Discard all loci that have any methylation in buffy coat. Number of loci with no buffy coat methylation is “low gdna bg loci.” There were more “low gdna bg loci” in pregnant samples compared to healthy controls, perhaps due to patient age differences in these subgroups.
2. In cfDNA, measure xbar: the mean number of molecules observed per locus, conditioned on the loci that had 1 or more molecule.
3. Of the low gdna bg loci, a fraction of them have methylation observed in the cfDNA. That is “frac methylated loci obs”. Healthy controls have methylation observed in only ~4% of the loci, whereas 20% of loci have methylated molecules in pregnancy.
4. Healthy controls have a “tumor fraction” of 0.4%, on average. This is due to methylation in the plasma that was not detected in buffy coat. The cfDNA tumor fraction increases as the stringency of gDNA background is relaxed, e.g. allowing more loci that have low levels of methylation in gDNA (data not shown). Thus, sequencing more buffy coat should reduce the tumor fraction observed in plasma.
5. Based on the estimated X, the total number of methylated loci in cfDNA can be inferred, even the ones that did not contribute an observed methylated molecule. This fit is not very robust, so while there were fewer methylated loci in healthy controls, the coefficient of variance is very high.
6. The “Total Methylation Fit” is conceptually analogous to TMS. It is the “methylated loci fit” multiplied by tumor fraction.

Results are shown in Table 11.

TABLE 11

| | healthy | | pregnant | | Overall | |
|---|---|---|---|---|---|---|
| | (N = 138) | | (N = 13) | | (N = 151) | |
| gdna total QE | | | | | | |
| Mean (SD) | 1060 | (300) | 783 | (175) | 1040 | (301) |
| Median [Q1-Q3] | 1020 | [801-1330] | 774 | [700-839] | 1000 | [770-1300] |
| cfdna total QE | | | | | | |
| Mean (SD) | 519 | (288) | 397 | (286) | 509 | (289) |
| Median [Q1-Q3] | 458 | [320-631] | 393 | [227-424] | 450 | [318-627] |
| low gdna | | | | | | |
| bg loci | | | | | | |
| Mean (SD) | 327 | (63.9) | 388 | (33.2) | 332 | (64.1) |
| Median [Q1-Q3] | 326 | [275-390] | 389 | [369-419] | 334 | [279-390] |
| xbar | | | | | | |
| Mean (SD) | 1.52 | (0.644) | 5.09 | (2.25) | 1.83 | (1.34) |
| Median [Q1-Q3] | 1.34 | [1.20-1.63] | 4.64 | [3.31-6.53] | 1.40 | [1.20-1.80] |
| tumor_fraction | | | | | | |
| Mean (SD) | 0.00434 | (0.00455) | 0.0359 | (0.0268) | 0.00738 | (0.0130) |
| Median [Q1-Q3] | 0.00328 | [0.00166-0.00496] | 0.0330 | [0.0127-0.0409] | 0.00388 | [0.00181-0.00572] |
| Missing | 16 | (11.6%) | 0 | (0%) | 16 | (10.6%) |
| frac methylated | | | | | | |
| loci obs | | | | | | |
| Mean (SD) | 0.0437 | (0.0255) | 0.223 | (0.0529) | 0.0592 | (0.0581) |
| Median [Q1-Q3] | 0.0365 | [0.0257-0.0562] | 0.225 | [0.199-0.239] | 0.0385 | [0.0262-0.0651] |
| methylated | | | | | | |
| loci fit | | | | | | |
| Mean (SD) | 36.8 | (32.7) | 89.2 | (20.7) | 41.8 | (35.3) |
| Median [Q1-Q3] | 26.4 | [16.0-46.6] | 88.6 | [83.1-95.2] | 29.3 | [16.7-57.1] |
| Missing | 16 | (11.6%) | 0 | (0%) | 16 | (10.6%) |
| Total Methylation | | | | | | |
| Fit | | | | | | |
| Mean (SD) | 0.111 | (0.109) | 3.25 | (2.62) | 0.413 | (1.22) |
| Median [Q1-Q3] | 0.0868 | [0.0474-0.124] | 3.15 | [1.23-3.90] | 0.0958 | [0.0522-0.141] |
| Missing | 16 | (11.6%) | 0 | (0%) | 16 | (10.6%) |

### Characteristics of Blank Samples

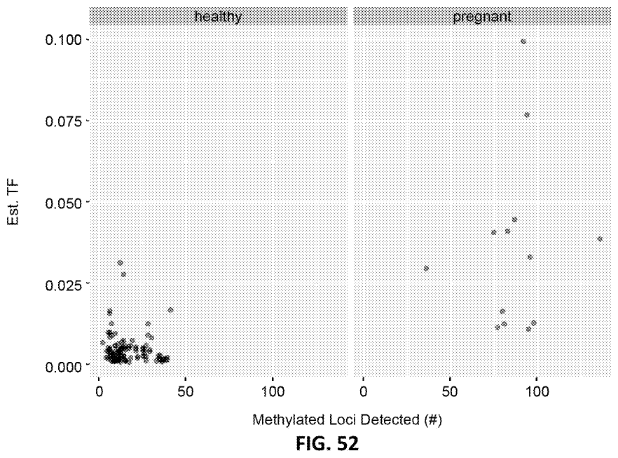

LOB is <50 methylated loci. Methylation fraction within the methylated loci in LOB samples can be as high as 1%. But, blanks are readily distinguished by how few methylated loci are found (FIG. 52).

### Analytical LOD

Two cancer tumors (breast cancer and non-small cell lung cancer) were obtained. They were shared and mixed into buffy coat at titrations ranging from 0.25% to 2%. 16 replicates were performed for each condition.

### LOD QE Count Distribution

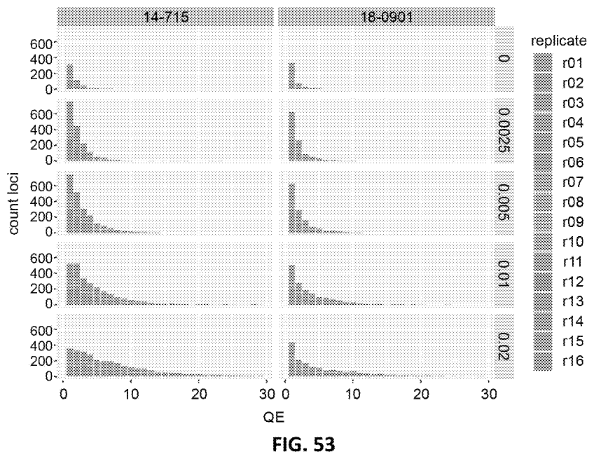

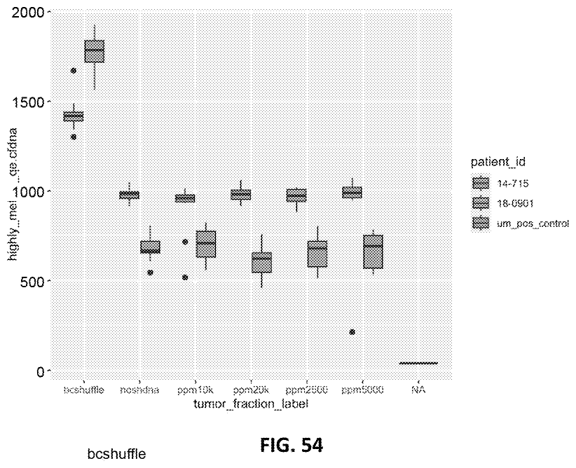

The frequency of observing 1,2, etc. molecules appear like a Poisson distribution (FIG. 53). The distribution of 18-0901 look different from 14-715, even at the same contrived tumor fraction. This is because patient 18-0901 had less DNA input (FIG. 54).

### Reproducing a TMS-Like Metric: Total Methylated Cell-Free DNA

The total methylated cell-free DNA is (methylated loci)*(tumor fraction). It should be very highly correlated with TMS.

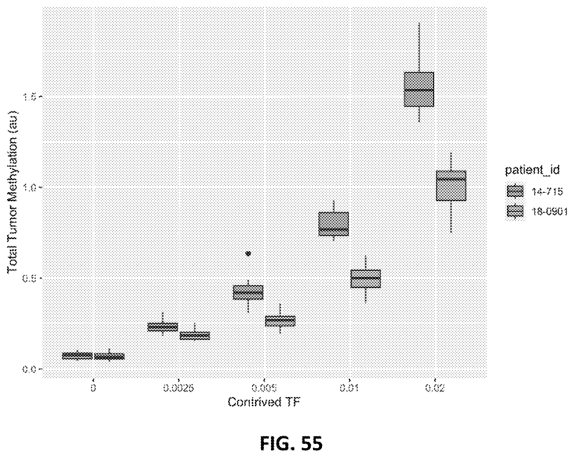

FIG. 55 shows the same results are reproduced. The two tumors are on different scales, but within a tumor, the total methylation is linear with the contrived tumor fraction.

### Methylated Tumor Fraction Recovers the Contrived Mixture Across Samples

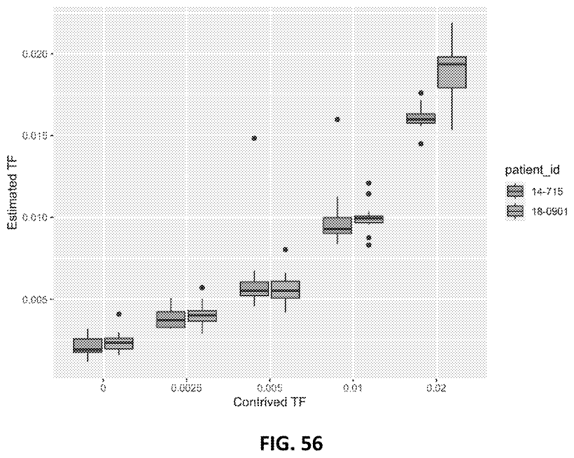

The tumor fraction successfully recovers the contrived input tumor fraction. However, this comes with a downside. The separation between contrived TF is less than the total methylation. Thus, total methylation and TMS have better discrimination ability for increase/decrease within a given tumor compared to tumor fraction (FIG. 56).

### Clinical Samples

Cancer samples that had both Northstar Response and Select performed on them were obtained. 236 samples were obtained from 65 patients. On average, there were 4.16 timepoints per patient. Sample characteristics are shown in Table 12.

TABLE 12
Clinical Samples

| | Overall (N = 236) | |
|---|---|---|
| tms | | |
| Mean (SD) | 10700 | (29700) |
| Median [Q1-Q3] | 171 | [49.8-4680] |
| max_vaf | | |
| Mean (SD) | 4.70 | (8.49) |
| Median [Q1-Q3] | 0.670 | [0.210-5.15] |
| num_t | | |
| Mean (SD) | 4.16 | (2.13) |
| Median [Q1-Q3] | 3.00 | [3.00-4.00] |

### Tumor Fraction Characteristics of Cancer Samples

Compared to healthy controls in the LOB, there are fewer low bg loci (171), but the fraction of observed methylated loci is much higher. The Total methylation is also much higher (Table 13).

TABLE 13

| | Overall (N = 236) | |
|---|---|---|
| gdna total QE | | |
| Mean (SD) | 1110 | (470) |
| Median [Q1-Q3] | 1070 | [866-1240] |
| cfdna total QE | | |
| Mean (SD) | 1010 | (405) |
| Median [Q1-Q3] | 995 | [664-1290] |
| tms | | |
| Mean (SD) | 10700 | (29700) |
| Median [Q1-Q3] | 171 | [49.8-4680] |
| max vaf | | |
| Mean (SD) | 4.70 | (8.49) |
| Median [Q1-Q3] | 0.670 | [0.210-5.15] |
| low gdna | | |
| bg loci | | |
| Mean (SD) | 171 | (55.9) |
| Median [Q1-Q3] | 170 | [128-206] |
| tumor_fraction | | |
| Mean (SD) | 0.0465 | (0.105) |
| Median [Q1-Q3] | 0.00283 | [0.00119-0.0372] |
| Missing | 23 | (9.7%) |
| frac methylated | | |
| loci obs | | |
| Mean (SD) | 0.349 | (0.265) |
| Median [Q1-Q3] | 0.247 | [0.132-0.568] |
| methylated | | |
| loci fit | | |
| Mean (SD) | 96.5 | (59.5) |
| Median [Q1-Q3] | 89.4 | [51.0-127] |
| Missing | 23 | (9.7%) |
| Total | | |
| Methylation Fit | | |
| Mean (SD) | 96.5 | (59.5) |
| Median [Q1-Q3] | 89.4 | [51.0-127] |
| Missing | 23 | (9.7%) |

### Correlation of TF Vs VAF is Comparable to TMS Vs VAF

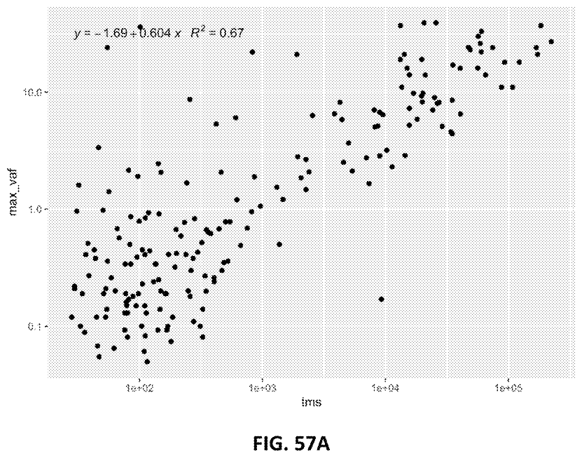

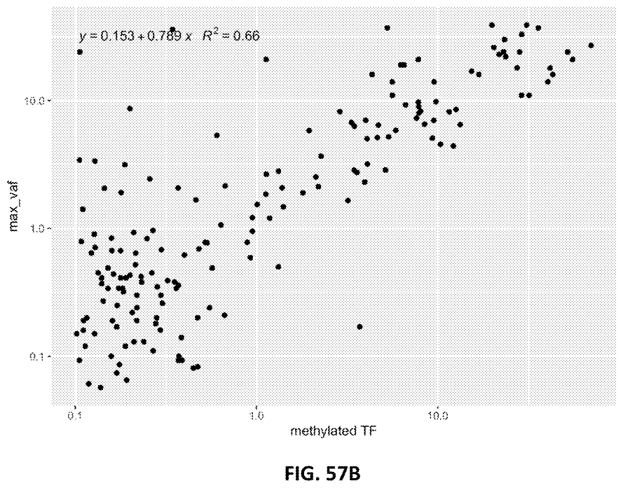

As a baseline, TMS was correlated with maxVAF in a sample. Restricting to samples with TMS>25, the R\hat{ }2 is 67%. (FIGS. 57A and 57B)

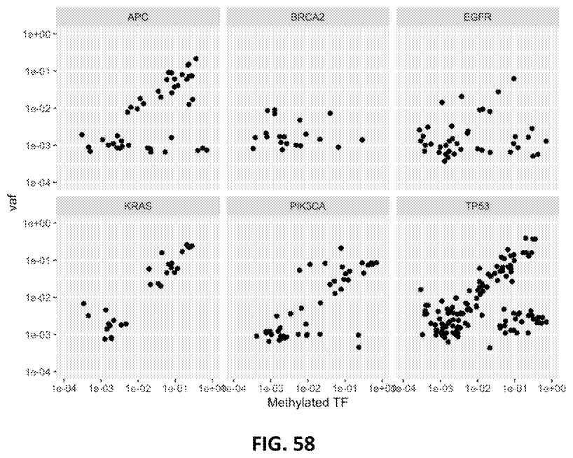

The correlation with VAF about the same, even though the measure of TF is more accurate now. There are distinct clusters of uncorrelated variants in genes like TP53 based on the association of methylated TF with SNPs (FIG. 58). Notably, KRAS variants are much better correlated with TF. This is explained by the confounding effect of clonal hematopoeisis for VAF-based measurements. TP53 is a commonly mutated in clonal hematopoeisis, which would explain the uncorrelated cluster. KRAS is rarely found in clonal hematopoesis, which explains the stronger correlation between VAF and methylated tumor fraction approaches in KRAS.

Many SNPs are at 100-Fold Lower VAF than Expected

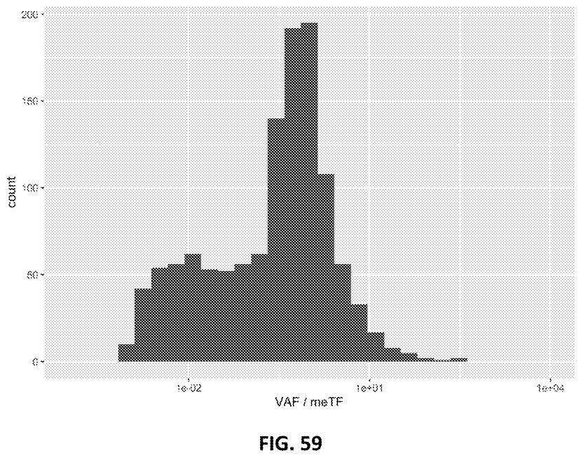

For each variant, the ratio of VAF with that sample's tumor fraction was calculated. As expected, there was a peak in the histogram around 1 (FIG. 59). However, there is also a prominent subpeak at 0.01.

### CONCLUSION

Computing the clipped mean/variance works down to lambda of 0.5. CV is ~15%

### TF Vs Methylated Loci

Result: The Ratiometric Method Tends to be Noisier than Simply Taking the Mean

Ratio of k=2/k=1 is best at 1<lambda <2.

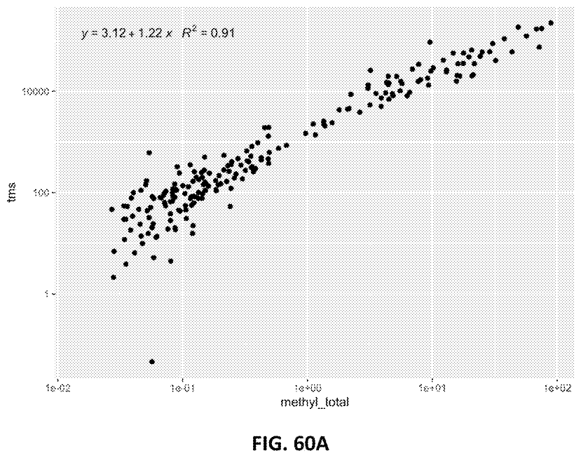

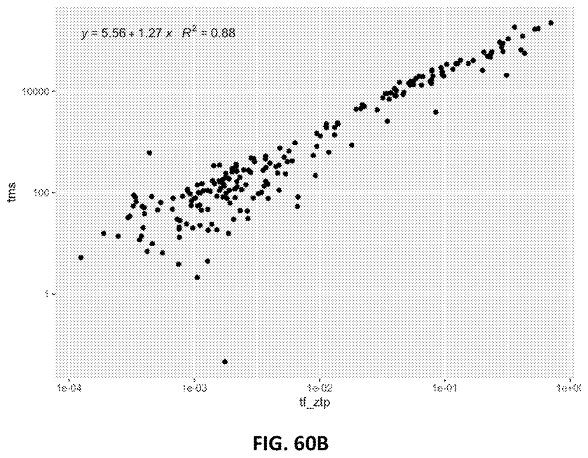

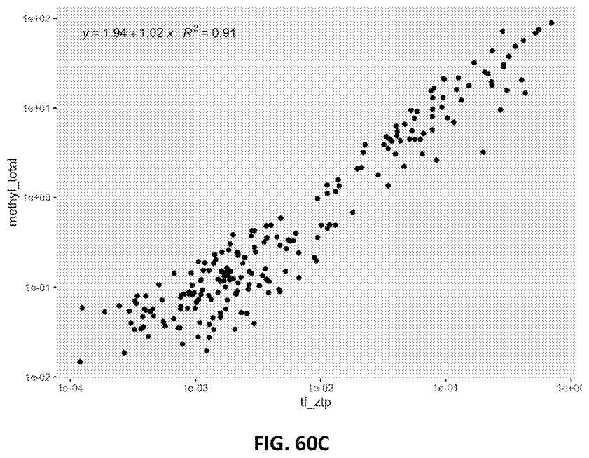

The correlation of “total methylation” with TMS is good with an, R²=88% (FIGS. 60A-60C).

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. For example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise embodiments described. The scope of protection should be limited only by any claims that ultimately issue.

1. A method of quantifying DNA methylation in a sample, comprising:
extracting DNA from the sample;
treating the DNA to encode presence or absence of DNA methylation in the DNA;
selectively enriching at least 100 loci of the DNA;
determining an absolute quantification of methylated DNA molecules at each locus; and
applying statistical analysis to a distribution of methylated DNA molecules across the loci to determine methylation fraction, wherein the statistical analysis comprises removing loci with zero methylated DNA molecules and calculating the methylation fraction based on a true mean of methylated DNA molecules per locus.

2. The method of claim 1, wherein the statistical analysis is zero-truncated Poisson statistical analysis.

3. The method of claim 1, wherein treating the sample to encode presence or absence of DNA methylation is conducted by bisulfite conversion or enzymatic conversion.

4. The method of claim 1, further comprising averaging the methylation fraction across all loci.

5. The method of claim 1, wherein at least 2000 CpG sites are amplified.

6. The method of claim 1, wherein determining the absolute quantification of methylated DNA molecules comprises sequencing amplicons generated by multiplex PCR at a read depth of at least one read sequence per molecule in the sample to generate sequence reads and determining a number of the sequence reads that are methylated sequence reads.

7. The method of claim 6, wherein an absolute number of unmethylated molecules in the sample is determined based on the number of methylated reads from the sample and a number of reads from a set of synthetic molecules that were co-amplified with the sample.

8. The method of claim 1, wherein a lower limit of detection is 0.1 molecules per locus.

9. The method of claim 1, wherein the loci are cancer-specific loci.

10. The method of claim 1, further comprising, prior to treating the sample to encode the presence or absence of DNA methylation,
(i) separating the sample into buffy coat and plasma;
(ii) extracting DNA from the buffy coat (white blood cell (WBC) DNA);
(iii) treating the WBC DNA to encode the presence or absence of DNA methylation in the WBC DNA; and
(iv) excluding loci that exhibit methylation in the WBC DNA sample from analysis of the cfDNA sample.

11. The method of claim 1, wherein the at least 100 loci comprise CpG sites.

12. A method of quantifying an abundance of a DNA modification in a sample, comprising:
extracting DNA from the sample
treating the DNA to encode presence or absence of DNA methylation in the DNA;
selectively enriching at least 100 loci of the DNA;
determining an absolute quantification of methylated DNA molecules at each locus;
applying statistical analysis to the distribution of methylated DNA molecules across the loci to determine methylation fraction wherein the statistical analysis comprises removing loci with zero methylated DNA molecules and calculating a true mean of methylated DNA molecules per locus; and
determining the count of the DNA modifications in the sample based on the true mean of methylated DNA molecules per locus.

13. The method of claim 12, wherein the DNA modification is a somatic mutation.

14. The method of claim 12, wherein the DNA modification is a copy number alteration.

15. A method of determining tumor fraction in a sample, comprising:
extracting DNA from the sample
treating the DNA to encode the presence or absence of DNA methylation in the DNA;
selectively enriching at least 100 loci of the DNA;
determining an absolute quantification of methylated DNA molecules at each locus;
applying statistical analysis to the distribution of methylated DNA molecules across the loci to determine methylation fraction wherein the statistical analysis comprises removing loci with zero methylated DNA molecules and calculating a true mean of methylated DNA molecules per locus; and
determining the tumor fraction in the sample based on the true mean of methylated DNA molecules per locus.

16. The method of claim 12, wherein the statistical analysis is zero-truncated Poisson statistical analysis.

17. The method of claim 12, wherein treating the sample to encode presence or absence of DNA methylation is conducted by bisulfite conversion or enzymatic conversion.

18. The method of claim 12, further comprising averaging the methylation fraction across all loci.

19. The method of claim 12, wherein at least 2000 DMR loci are amplified.

20. The method of claim 12, wherein determining the absolute quantification of methylated DNA molecules comprises sequencing amplicons generated by multiplex PCR at a read depth of at least one read sequence per molecule in the sample to generate sequence reads and determining a number of the sequence reads that are methylated sequence reads.

21. The method of claim 20, wherein an absolute number of unmethylated molecules in the sample is determined based on the number of methylated reads from the sample and a number of reads from a set of synthetic molecules that were co-amplified with the sample.

22. The method of claim 12, wherein the lower limit of detection is 0.1 molecules per locus.

23. The method of claim 12, wherein the at least 100 loci are cancer-specific loci.

24. The method of claim 12, further comprising, prior to treating the sample to encode the presence or absence of DNA methylation,
(i) separating the sample into buffy coat and plasma;
(ii) extracting DNA from the buffy coat (white blood cell (WBC) DNA);
(iii) treating the WBC DNA to encode the presence or absence of methylation in the WBC DNA; and
(iv) excluding loci that exhibit methylation in the WBC DNA sample from analysis of the cfDNA sample.

25. The method of claim 15, wherein the statistical analysis is zero-truncated Poisson statistical analysis.

26. The method of claim 15, wherein treating the sample to encode presence or absence of DNA methylation is conducted by bisulfite conversion or enzymatic conversion.

27. The method of claim 15, further comprising averaging the methylation fraction across all loci.

28. The method of claim 15, wherein at least 2000 DMR loci are amplified.

29. The method of claim 15, wherein determining the absolute quantification of methylated DNA molecules comprises sequencing amplicons generated by multiplex PCR at a read depth of at least one read sequence per molecule in the sample to generate sequence reads and determining a number of the sequence reads that are methylated sequence reads.

30. The method of claim 29, wherein an absolute number of unmethylated molecules in the sample is determined based on the number of methylated reads from the sample and a number of reads from a set of synthetic molecules that were co-amplified with the sample.

31. The method of claim 15, wherein the lower limit of detection is 0.1 molecules per locus.

32. The method of claim 15, wherein the at least 100 loci are cancer-specific loci.

33. The method of claim 15, further comprising, prior to treating the sample to encode the presence or absence of DNA methylation,
(i) separating the sample into buffy coat and plasma;
(ii) extracting DNA from the buffy coat (white blood cell (WBC) DNA);
(iii) treating the WBC DNA to encode the presence or absence of methylation in the WBC DNA; and
(iv) excluding loci that exhibit methylation in the WBC DNA sample from analysis of the cfDNA sample.
